(19) United States
(12) Patent Application Publication
(10) Pub. No.: US 2013/0178475 A1
(43) Pub. Date: Jul. 11, 2013

(54) sGC STIMULATORS

(71) Applicants: Joel Moore, Lexington, MA (US); James Jia, Somerville, MA (US); Takashi Nakai, Newton, MA (US); Charles Kim, Cambridge, MA (US); Thomas Wai-Ho Lee, Lexington, MA (US); Jane Yang, Andover, MA (US)

(72) Inventors: Joel Moore, Lexington, MA (US); James Jia, Somerville, MA (US); Takashi Nakai, Newton, MA (US); Charles Kim, Cambridge, MA (US); Thomas Wai-Ho Lee, Lexington, MA (US); Jane Yang, Andover, MA (US)

(73) Assignee: IRONWOOD PHARMACEUTICALS, INC., Cambridge, MA (US)

(21) Appl. No.: 13/635,597

(22) PCT Filed: Mar. 10, 2011
(86) PCT No.: PCT/US2011/027824

§ 371 Date: Mar. 26, 2013

Related U.S. Application Data
(60) Provisional application No. 61/314,966, filed on Mar. 17, 2010.
(60) Provisional application No. 61/446,777, filed on Feb. 25, 2011.

(51) Int. Cl. C07D 495/04; C07D 401/14; C07D 403/04; C07D 413/14; C07D 471/04; C07D 401/04

(52) U.S. Cl. CPC C07D 495/04; C07D 471/04; C07D 401/04; C07D 403/04; C07D 413/14; C07D 401/14
USPC 514/245; 514/256; 514/303; 514/338; 544/207; 544/296; 544/333; 546/119; 546/275.7

(57) ABSTRACT

Compounds of Formula I are described. They are useful as stimulators of sGC, particularly NO-independent, heme-dependent stimulators. These compounds may be useful for treating, preventing or managing various disorders that are herein disclosed.

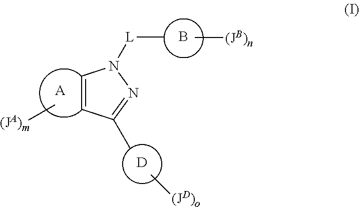

This patent application claims the benefits of U.S. Provisional Application Nos. 61/314,966 filed Mar. 17, 2010 and 61/446,777 filed Feb. 25, 2011, the disclosures of which are incorporated by reference.

### FIELD OF THE INVENTION

The present disclosure relates to stimulators of soluble guanylate cyclase (sGC), pharmaceutical formulations thereof and their use, alone or in combination with one or more additional agents, for treating and/or preventing various diseases, wherein an increase in the concentration of NO might be desirable.

### BACKGROUND OF THE INVENTION

Soluble guanylate cyclase (sGC) is the primary receptor for nitric oxide (NO) in vivo. sGC can be activated via both NO-dependent and NO-independent mechanisms. In response to this activation, sGC converts GTP into the secondary messenger cyclic GMP (cGMP). The increased level of cGMP, in turn, modulates the activity of downstream effectors including protein kinases, phosphodiesterases (PDEs), and ion channels.

In the body, NO is synthesized from arginine and oxygen by various nitric oxide synthase (NOS) enzymes and by sequential reduction of inorganic nitrate. Three distinct isoforms of NOS have been identified: inducible NOS (iNOS or NOS II) found in activated macrophage cells; constitutive neuronal NOS (nNOS or NOS I), involved in neurotransmission and long term potentiation; and constitutive endothelial NOS (eNOS or NOS III) which regulates smooth muscle relaxation and blood pressure.

Experimental and clinical evidence indicates that reduced bioavailability and/or responsiveness to endogenously produced NO contributes to the development of cardiovascular, endothelial, renal and hepatic disease, as well as erectile dysfunction. In particular, the NO signaling pathway is altered in cardiovascular diseases, including, for instance, systemic and pulmonary hypertension, heart failure, stroke, thrombosis and atherosclerosis.

Pulmonary hypertension (PH) is a disease characterized by sustained elevation of blood pressure in the pulmonary vasculature (pulmonary artery, pulmonary vein and pulmonary capillaries), which results in right heart hypertrophy, eventually leading to right heart failure and death. In PH, the bioactivity of NO and other vasodilators such as prostacyclin is reduced, whereas the production of endogenous vasoconstrictors such as endothelin is increased, resulting in excessive pulmonary vasoconstriction. sGC stimulators have been used to treat PH because they promote smooth muscle relaxation, which leads to vasodilation.

Treatment with NO-independent sGC stimulators also promoted smooth muscle relaxation in the corpus cavernosum of healthy rabbits, rats and humans, causing penile erection, indicating that sGC stimulators are useful for treating erectile dysfunction.

NO-independent, heme-dependent, sGC stimulators, such as those disclosed herein, have several important differentiating characteristics, including crucial dependency on the presence of the reduced prosthetic heme moiety for their activity, strong synergistic enzyme activation when combined with NO and stimulation of the synthesis of cGMP by direct stimulation of sGC, independent of NO. The benzylindazole compound YC-1 was the first sGC stimulator to be identified. Additional sGC stimulators with improved potency and specificity for sGC have since been developed. These compounds have been shown to produce anti-aggregratory, anti-proliferative and vasodilatory effects.

Since compounds that stimulate sGC in an NO-independent manner offer considerable advantages over other current alternative therapies, there is a need to develop novel stimulators of sGC, because they would be useful in the prevention, management and treatment of disorders such as pulmonary hypertension, arterial hypertension, heart failure, atherosclerosis, inflammation, thrombosis, renal fibrosis and failure, liver cirrhosis, erectile dysfunction and other cardiovascular disorders.

### SUMMARY OF THE INVENTION

The present invention is directed to compounds according to Formula I, or a pharmaceutically acceptable salt thereof,

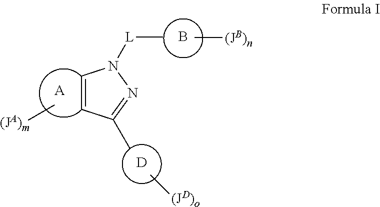

wherein:
ring A is selected from a 5 to 10-membered cycloaliphatic ring or a 5 to 10-membered non-aromatic heterocycle; wherein said heterocycle contains from 1 to 3 heteroatoms independently selected from N, O or S, or alternatively said heterocycle contains from 1 to 3 heteroatoms independently selected from O or S;
m is an integer selected from 0 to 3;
if J^{A} is a substituent on a ring carbon atom, J^{A} is independently selected from halogen, —CN, —NO₂, a C₁₋₆ aliphatic, —OR^{A}, —SR^{A}, —COR^{A}, —C(O)OR^{A}, —C(O)N(R^{A})₂, —N(R^{A})₂, —N(R^{A})C(O)R^{a}, —N(R^{A})C(O)OR^{a}, —SO₂R^{A}, —SO₂N(R^{A})₂, —N(R^{A})SO₂R^{a}, —N(R^{A})SO₂N(R^{a})₂, a C₃₋₈ cycloaliphatic ring, a 4 to 8-membered heterocyclic ring, a 5 to 6-membered heteroaryl ring or an oxo group; wherein each said C₁₋₆ aliphatic, said C₃₋₈ cycloaliphatic ring, said 4 to 8-membered heterocyclic ring and said 5 to 6-membered heteroaryl ring is independently substituted with from 0 to 3 substituents selected from halogen, —OH, —O(C₁₋₄ alkyl), —O(C₁₋₄ haloalkyl), —NH₂, —N(C₁₋₄ alkyl)₂, —NH(C₁₋₄ alkyl), —COOH, —NO₂, —CN or an oxo group;
if J^{A} is a substituent on a ring nitrogen atom, when present, J^{A} is independently selected from —C(O)R^{A}, —C(O)OR^{A}, —C(O)N(R^{A})₂, —SO₂R^{A}, —SO₂N(R^{A})₂, C₁₋₆ aliphatic, —(C₁₋₆ aliphatic)-R^{A}, a C₃₋₈ cycloaliphatic ring, a 6 or 10-membered aryl ring, a 4 to 8-membered heterocyclic ring, or a 5 to 6-membered heteroaryl ring; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 6 or 10-membered aryl ring, said 4 to 8-membered heterocyclic ring and said 5 to 6-membered heteroaryl ring is independently substituted with from 0 to 3 instances of R¹;
each R^{A} is independently selected from hydrogen, C₁₋₆ aliphatic, a C₃₋₈ cycloaliphatic ring, a 4 to 8-membered heterocyclic ring, phenyl or a 5 to 6-membered heteroaryl ring; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted with from 0 to 3 instances of R¹;
each R^{a} is independently selected from hydrogen, C₁₋₆ aliphatic, a C₃₋₈ cycloaliphatic ring, a 4 to 8-membered heterocyclic ring, phenyl or a 5 to 6-membered heteroaryl ring; wherein each said heterocylic ring and said heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl rings is independently substituted by from 0 to 3 instances of R¹;
   each R¹ is independently selected from halogen, —CN, C₁₋₄ alkyl, C₁₋₄ haloalkyl, —OR², —SR², —COR², —C(O)OR², —C(O)N(R²)₂, —N(R²)C(O)R², —N(R²)₂, —SO₂R², —SO₂N(R²)₂, —N(R)SO₂R, phenyl or an oxo group, wherein said phenyl group is optionally substituted with from 0 to 3 substituents independently selected from halogen, hydroxy, —NH₂, —NH(C₁₋₄ alkyl), —N(C₁₋₄ alkyl)₂, —NO₂, —CN, C₁₋₄ alkyl, C₁₋₄ haloalkyl, C₁₋₄ alkoxy or —O(C₁₋₄ haloalkyl);
      each R² is independently selected from hydrogen, a C₁₋₄ alkyl, phenyl, benzyl or C₃₋₈ cycloalkyl group, each of said C₁₋₄ alkyl, phenyl, benzyl and C₃₋₈ cycloalkyl group independently substituted by from 0 to 3 instances of halogen; or alternatively two R² groups attached to the same nitrogen atom, together with said nitrogen atom may form a 5 to 8-membered heterocyclic ring or a 5-membered heteroaryl ring; each said 5 to 8-membered heterocyclic ring and said 5-membered heteroaryl ring containing 1 or 2 additional heteroatoms independently selected from N, O or S;
if J^{A} is a substituent on a ring sulfur atom, when present, J^{A} is oxo;
or, alternatively, two J^{A} groups attached to two non-vicinal ring atoms of ring A, together with said non-vicinal atoms, form a C₅₋₈ carbocyclic ring or a 5 to 8-membered heterocyclic ring with said two J^{A} groups forming a bridge for ring A between the two non-vincinal ring atoms; wherein said 5 to 8-membered heterocyclic ring contains 1 or 2 heteroatoms independently selected from N, S or O, or alternatively said 5 to 8-membered heterocyclic ring contains 1 or 2 heteroatoms independently selected from S or O; and wherein said C₅₋₈ carbocyclic ring or 5 to 8-membered heterocyclic ring formed by said two J^{A} groups is optionally and independently substituted with from 0 to 2 substituents selected from halogen, hydroxy, —NH₂, —NH(C₁₋₄ alkyl), —N(C₁₋₄ alkyl)₂, —NO₂, —CN, C₁₋₄ alkyl, C₁₋₄ alkoxy, C₁₋₄ haloalkyl or C₁₋₄ haloalkoxy groups;
L is a methylene linker, independently substituted by from 0 to 2 substituents selected from halogen or C₁₋₆ alkyl, wherein when two substituents on the methylene linker are C₁ alkyl groups, the two C₁ alkyl groups together with the carbon atom to which the two C₁ alkyl groups are attached may form a cyclopropyl ring; wherein each said C₁₋₆ alkyl and said cyclopropyl is optionally and independently substituted by from 0 to 3 instances of halogen;
ring B is selected from a monocyclic or bicyclic 6 to 10-membered aryl or a 6 to 10-membered heteroaryl; wherein said 6 to 10-membered heteroaryl contains from 1 to 4 heteroatoms independently selected from N, O or S;
n is an integer selected from 0 to 3;
if J^{B} is a substituent on a ring carbon atom, J^{B} is independently selected from halogen, —CN, —NO₂, a C₁₋₆ aliphatic, —OR^{B}, —SR^{B}, —COR^{B}, —C(O)OR^{B}, —C(O)N(R^{B})₂, —N(R^{B})₂, —N(R^{B})C(O)R^{b}, —N(R^{B})C(O)OR^{b}, —SO₂R^{B}, —SO₂N(R^{B})₂, —N(R^{B})SO₂R^{b}, —N(R^{B})SO₂N(R^{b})₂, a C₃₋₈ cycloaliphatic group, a 4 to 8-membered heterocyclic group, a 5 to 6-membered heteroaryl group or an oxo group; wherein each said C₁₋₆ aliphatic, said C₃₋₈ cycloaliphatic group, said 4 to 8-membered heterocyclic group and said 5 to 6-membered heteroaryl group is independently substituted with from 0 to 3 substituents selected from halogen, —OH, C₁₋₄ alkyl, C₁₋₄ haloalkyl, —O(C₁₋₄ alkyl), —O(C₁₋₄ haloalkyl), —NH₂, —N(C₁₋₄ alkyl)₂, —NH(C₁₋₄ alkyl), —COOH, —CN, —NO₂ or oxo;
if J^{B} is a substituent on a ring nitrogen atom, when present, J^{B} is independently selected from —C(O)R^{B}, —C(O)OR^{B}, —C(O)N(R^{B})₂, —SO₂R^{B}, —SO₂N(R^{B})₂, a C₁₋₆ aliphatic, a —(C₁₋₆ aliphatic)-R^{B}, a C₃₋₈ cycloaliphatic ring, a 4 to 8-membered heterocyclic ring, or a 5 to 6-membered heteroaryl ring; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring and said 5 to 6-membered heteroaryl ring is independently substituted with from 0 to 3 instances of R³;
or, alternatively, two J^{B} groups attached to two vicinal ring B atoms, taken together with said two vicinal ring B atoms, form a 5 to 7-membered heterocycle resulting in a fused ring B; wherein said 5 to 7-membered heterocycle contains from 1 to 2 heteroatoms independently selected from N, O or S; and wherein said 5 to 7-membered heterocycle is optionally substituted by from 0 to 3 substituents independently selected from halogen, hydroxy, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl);
each R^{B} is independently selected from hydrogen, a C₁₋₆ aliphatic, a C₃₋₈ cycloaliphatic ring, a 4 to 8-membered heterocyclic ring, phenyl or a 5 to 6-membered heteroaryl ring; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted with from 0 to 3 instances of R³;
each R^{b} is independently selected from hydrogen, a C₁₋₆ aliphatic, a C₃₋₈ cycloaliphatic ring, a 4 to 8-membered heterocyclic ring, phenyl or a 5 to 6-membered heteroaryl ring; wherein each said heterocylic ring and said heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl rings is independently substituted by from 0 to 3 instances of R³;
   each R³ is independently selected from halogen, —CN, C₁₋₄ alkyl, C₁₋₄ haloalkyl, —OR⁴, —SR⁴, —COR⁴, —C(O)OR⁴, —C(O)N(R⁴)₂, —N(R⁴)C(O)R⁴, —N(R⁴)₂, —SO₂R⁴, —SO₂N(R⁴)₂, —N(R⁴)SO₂R⁴, phenyl or an oxo group, wherein said phenyl group is optionally substituted with from 0 to 3 substituents independently selected from halogen, hydroxy, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl);
      each R⁴ is independently selected from hydrogen, a C₁₋₄ alkyl, phenyl, benzyl or C₃₋₈ cycloalkyl group, each of said C₁₋₄ alkyl, phenyl, benzyl or cycloalkyl groups independently substituted by from 0 to 3 instances of halogen; or alternatively two R⁴ groups attached to the same nitrogen atom, together with said nitrogen atom may form a 5 to 8-membered heterocyclic ring or a 5-membered heteroaryl ring; each said 5 to 8-membered heterocyclic ring and said 5-membered heteroaryl ring containing 1 or 2 additional heteroatoms independently selected from N, O or S;
ring D is a 6-membered heteroaryl which contains from 1 to 3 instances of N;
o is an integer selected from 0 to 3;
if J^{D} is a substituent on a ring carbon atom, it is independently selected from halogen, —NO₂, oxo, —OR^{D}, —C(O)R^{D}, —C(O)OR^{D}, —C(O)N(R^{D})₂, —CN, —N(R^{D})₂, —N═NR^{D}, —N(R^{D})C(O)R^{d}, —N(R^{D})C(O)OR^{d}, —SO₂R^{D}, —SO₂N(R^{D})₂, —N(R^{D})SO₂R^{d}, C₁₋₆ aliphatic, —(C₁₋₆ aliphatic)-R^{D}, a C₃₋₈ cycloaliphatic ring, a 6 or 10-membered aryl ring, 4 to 8-membered heterocyclic ring or a 5 to 6-membered heteroaryl; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 6 or 10-membered aryl ring, said 4 to 8-membered heterocyclic ring and said 5 to 6-membered heteroaryl ring is independently substituted with from 0 to 3 instances of R⁵;
each R^{D} is independently selected from hydrogen, a C₁₋₆ aliphatic, a C₃₋₈ cycloaliphatic ring, a 4 to 8-membered heterocyclic ring, phenyl or a 5 to 6-membered heteroaryl ring; wherein each said 4 to 8-membered heterocylic and said 5 to 6-membered heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted with from 0 to 3 instances of R⁵;
each R^{d} is independently selected from hydrogen, a C₁₋₆ aliphatic, a C₃₋₈ cycloaliphatic ring, a 4 to 8-membered heterocyclic ring, phenyl or a 5 to 6-membered heteroaryl ring; wherein each said heterocylic ring and said heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted by from 0 to 3 instances of R⁵;
   each R⁵ is independently selected from halogen, —CN, C₁₋₄ alkyl, C₁₋₄ haloalkyl, —OR⁶, —SR⁶, —COR^{E}, —C(O)OR⁶, —C(O)N(R⁶)₂, —N(R⁶)C(O)R⁶, —N(R⁶)₂, —SO₂R⁶, —SO₂N(R⁶)₂, —N(R⁶)SO₂R⁶, phenyl or an oxo group, wherein said phenyl group is optionally substituted with from 0 to 3 substituents independently selected from halogen, hydroxy, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl);
   each R⁶ is independently selected from hydrogen, a C₁₋₄ alkyl, phenyl, benzyl or a C₃₋₈ cycloalkyl group, wherein each of said C₁₋₄ alkyl, said phenyl, said benzyl and said cycloalkyl group is independently substituted by from 0 to 3 instances of halogen; or alternatively two R⁶ groups attached to the same nitrogen atom, together with said nitrogen atom form a 5 to 8-membered heterocyclic ring or a 5-membered heteroaryl ring; each said 5 to 8-membered heterocyclic ring and said 5-membered heteroaryl ring containing 1 or 2 additional heteroatoms independently selected from N, O or S;
or, alternatively, two J^{D} groups attached to two vicinal ring D atoms, taken together with said two vicinal ring D atoms, form a 5 to 7-membered heterocycle resulting in a fused ring D wherein said 5 to 7-membered heterocycle contains from 1 to 3 heteroatoms independently selected from N, O or S; and wherein said 5 to 7-membered heterocycle is optionally and independently substituted by from 0 to 3 substituents selected from halogen, hydroxy, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl);
provided that the compound according to Formula I is not:

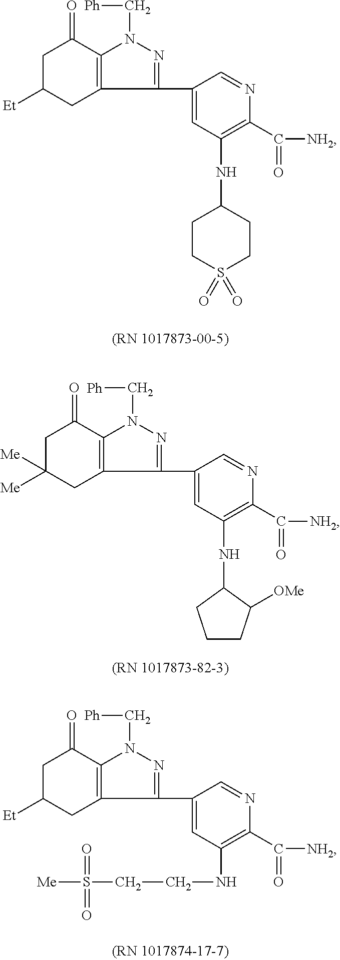 [SMILES: CCC1CC(=O)c2c(c(-c3cnc(C(N)=O)c(NC4CCS(=O)(=O)CC4)c3)nn2Cc2ccccc2)C1 ; COC1CCCC1Nc1cc(-c2nn(Cc3ccccc3)c3c2CC(C)(C)CC3=O)cnc1C(N)=O ; CCC1CC(=O)c2c(c(-c3cnc(C(N)=O)c(NCCS(C)(=O)=O)c3)nn2Cc2ccccc2)C1]

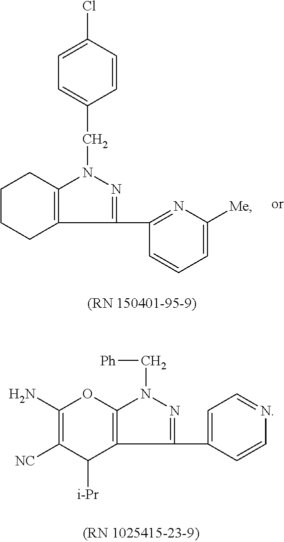 [SMILES: Cc1cccc(-c2nn(Cc3ccc(Cl)cc3)c3c2CCCC3)n1 ; CC(C)C1C(C#N)=C(N)Oc2c1c(-c1ccncc1)nn2Cc1ccccc1]

The invention also provides a method of treating a disease, health condition or disorder in a subject in need of the treatment, comprising administering a therapeutically effective amount of the compound of Formula I or a pharmaceutically acceptable salt thereof to the subject, wherein the disease, health condition or disorder is a peripheral or cardiac vascular disorder/condition, or a urogenital system disorder that can benefit from sGC stimulation.

### DETAILED DESCRIPTION OF THE INVENTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### DEFINITIONS AND GENERAL TERMINOLOGY

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

As described herein, compounds of Formula I may be optionally substituted with one or more substituents, such as illustrated generally below, or as exemplified by particular classes, subclasses, and species of the invention. The phrase “optionally substituted” is used interchangeably with the phrase “substituted or unsubstituted.” In general, the term “substituted”, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. If a substituent radical or structure is not identified or defined as “optionally substituted”, the substituent radical or structure is not substituted. As will be apparent to one of ordinary skill in the art, groups such as —H, halogen, —NO₂, —CN, —OH, —NH₂ or —OCF₃ would not be substitutable groups.

The phrase “up to”, as used herein, refers to zero or any integer number that is equal or less than the number following the phrase. For example, “up to 3” means any one of 0, 1, 2, or 3. As described herein, a specified number range of atoms includes any integer therein. For example, a group having from 1-4 atoms could have 1, 2, 3 or 4 atoms. It will be understood by one of ordinary skill in the art that when a group is characterized as substituted (as opposed to optionally substituted) with, e.g., “up to 3” substituents, it can only be substituted with 1, 2 or 3 substituents.

When any variable occurs more than one time at any position, its definition on each occurrence is independent from every other occurrence.

Selection of substituents and combinations envisioned by this disclosure are only those that result in the formation of stable or chemically feasible compounds. Such choices and combinations will be apparent to those of ordinary skill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. In some embodiments, a stable compound or chemically feasible compound is one that is not substantially altered when kept at a temperature of 25° C. or less, in the absence of moisture or other chemically reactive conditions, for at least a week.

A compound, such as the compounds of Formula I or other compounds herein disclosed, may be present in its free form (e.g. an amorphous form, a crystalline form or polymorphs). Under certain conditions, compounds may also form salts, and/or other multi-component crystalline forms (e.g. solvates, hydrates and co-crystals). As used herein, the term co-form is synonymous with the term multi-component crystalline form. When one of the components in the co-form has clearly transferred a proton to the other component, the resulting co-form is referred to as a “salt”. When both compounds in a multi-component crystalline form are independently solids at room temperature, the resulting co-form is referred to as a “co-crystal”. In co-crystals no proton transfer takes place between the different components of the co-form. The formation of a salt or a co-crystal is determined by how large the difference is in the pKas between the partners that form the mixture.

As used herein, a “solvate” refers to an association or complex of one or more solvent molecules and a compound disclosed herein (or its salts or co-crystals). A “hydrate” is a particular type of solvate in which the solvent is water. Examples of solvents that can form solvates include, but are not limited to: water, isopropanol, ethanol, methanol, (dimethyl sulfoxide) DMSO, ethyl acetate, acetic acid, ethanolamine, tetrahydrofuran (THF), dichloromethane (DCM), N,N-dimethylformamide (DMF).

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are within the scope of the disclosure.

The present disclosure also embraces isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The term “aliphatic” or “aliphatic group”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”. In some instances the term can be used in the phrase “aromatic carbocycle”, and in this case it refers to an “aryl group” as defined below.

The term “cycloaliphatic” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, spiro or bridged. In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3-7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

“Heterocycle” (or “heterocyclyl” or “heterocyclic), as used herein, refers to a ring system in which one or more ring members are an independently selected heteroatom, which is completely saturated or that contains one or more units of unsaturation but which is not aromatic, and which has a single point of attachment to the rest of the molecule. Unless otherwise specified, through this disclosure, heterocycle is used as a synonym of “non-aromatic heterocycle”. In some instances the term can be used in the phrase “aromatic heterocycle”, and in this case it refers to a “heteroaryl group” as defined below. The term heterocycle also includes fused, spiro or bridged heterocyclic ring systems. Unless otherwise specified, a heterocycle may be monocyclic, bicyclic or tricyclic. In some embodiments, the heterocycle has 3-18 ring members in which one or more ring members is a heteroatom independently selected from oxygen, sulfur or nitrogen, and each ring in the system contains 3 to 7 ring members. In other embodiments, a heterocycle may be a monocycle having 3-7 ring members (2-6 carbon atoms and 1-4 heteroatoms) or a bicycle having 7-10 ring members (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is on the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6,5 heteroaromatic system, as used herein, for example, is a six membered heteroaromatic ring fused to a second five membered ring wherein the radical or point of attachment is on the six membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tri-cyclic ring systems including cycloaliphatic, heterocyclic, aryl or heteroaryl, each of which has been previously defined.

“Fused” bicyclic ring systems comprise two rings which share two adjoining ring atoms.

“Bridged” bicyclic ring systems comprise two rings which share three or four adjacent ring atoms. As used herein, the term “bridge” refers to a bond or an atom or a chain of atoms connecting two different parts of a molecule. The two atoms that are connected through the bridge (usually but not always, two tertiary carbon atoms) are referred to as “bridgeheads”. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl.

“Spiro” bicyclic ring systems share only one ring atom (usually a quaternary carbon atom).

The term “ring atom” refers to an atom such as C, N, O or S that is part of the ring of an aromatic group, a cycloaliphatic group or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example N (as in 3,4-dihydro-2H-pyrrolyl), NH (as in pyrrolidinyl) or NR⁺ (as in N-substituted pyrrolidinyl).

In some embodiments, two independent occurrences of a variable may be taken together with the atom(s) to which each variable is bound to form a 5-8-membered, heterocyclyl, aryl, or heteroaryl ring or a 3-8-membered cycloalkyl ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of R^{o} as in Formula D1:

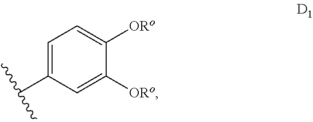

these two occurrences of R^{o} are taken together with the oxygen atoms to which they are bound to form a fused 6-membered oxygen containing ring as in Formula D2:

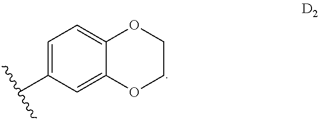 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

In some embodiments, an alkyl or aliphatic chain can be optionally interrupted with another atom or group. This means that a methylene unit of the alkyl or aliphatic chain can optionally be replaced with said other atom or group. Unless otherwise specified, the optional replacements form a chemically stable compound. Optional interruptions can occur both within the chain and/or at either end of the chain; i.e. both at the point of attachment(s) to the rest of the molecule and/or at the terminal end. Two optional replacements can also be adjacent to each other within a chain so long as it results in a chemically stable compound. Unless otherwise specified, if the replacement or interruption occurs at a terminal end of the chain, the replacement atom is bound to a H on the terminal end. For example, if —CH₂CH₂CH₃ were optionally interrupted with —O—, the resulting compound could be —OCH₂CH₃, —CH₂OCH₃, or —CH₂CH₂OH. In another example, if the divalent linker —CH₂CH₂CH₂-were optionally interrupted with —O—, the resulting compound could be —OCH₂CH₂—, —CH₂OCH₂—, or —CH₂CH₂O—. The optional replacements can also completely replace all of the carbon atoms in a chain. For example, a C₃ aliphatic can be optionally replaced by —N(R′)—, —C(O)—, and —N(R′)— to form —N(R′)C(O)N(R′)— (a urea).

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{x}O(O)C-alkyl is an example of a carboxy group used terminally. A group is internal when the group is present in the middle of a substituent at the end of the substituent bound to the rest of the chemical structure. Alkylcarboxy (e.g., alkyl-C(O)O— or alkyl-O(CO)—) and alkylcarboxyaryl (e.g., alkyl-C(O)O-aryl- or alkyl-O(CO)-aryl-) are examples of carboxy groups used internally.

As described herein, a bond drawn from a substituent to the center of one ring within a multiple-ring system (as shown below), represents substitution of the substituent at any substitutable position in any of the rings within the multiple ring system. For example, formula D3 represents possible substitution in any of the positions shown in formula D4:

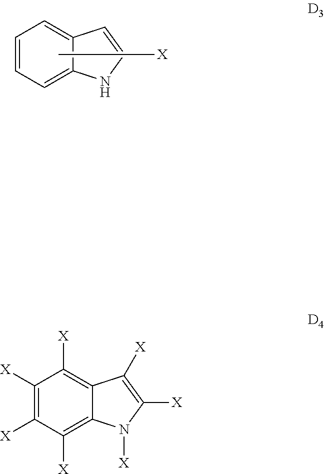

This also applies to multiple ring systems fused to optional ring systems (which would be represented by dotted lines). For example, in Formula D5, X is an optional substituent both for ring A and ring B.

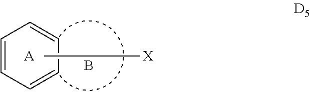

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

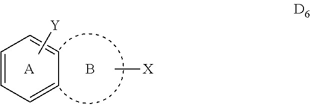

As used herein, the terms “alkoxy” or “alkylthio” refer to an alkyl group, as previously defined, attached to the molecule, or to another chain or ring, through an oxygen (“alkoxy” i.e, —O-alkyl) or a sulfur (“alkylthio” i.e., —S-alkyl) atom.

The terms Cₙ₋ₘ “alkoxyalkyl”, Cₙ₋ₘ “alkoxyalkenyl”, Cₙ₋ₘ “alkoxyaliphatic”, and Cₙ₋ₘ “alkoxyalkoxy” mean alkyl, alkenyl, aliphatic or alkoxy, as the case may be, substituted with one or more alkoxy groups, wherein the combined total number of carbons of the alkyl and alkoxy groups, alkenyl and alkoxy groups, aliphatic and alkoxy groups or alkoxy and alkoxy groups, combined, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —CH₂CH₂CH₂OCH₃.

When the moieties described in the preceding paragraph are optionally substituted, they can be substituted in either or both of the portions on either side of the oxygen or sulfur. For example, an optionally substituted C₄ alkoxyalkyl could be, for instance, —CH₂CH₂OCH₂(Me)CH₃ or —CH₂(OH)OCH₂CH₂CH₃; a C₅ alkoxyalkenyl could be, for instance, —CH═CHOCH₂CH₂CH₃ or —CH═CHCH₂OCH₂CH₃.

The terms aryloxy, arylthio, benzyloxy or benzylthio, refer to an aryl or benzyl group attached to the molecule, or to another chain or ring, through an oxygen (“aryloxy”, benzyloxy e.g., —O-Ph, —OCH₂Ph) or sulfur (“arylthio” e.g., —S-Ph, —S—CH₂Ph) atom. Further, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or aliphatic will be indicated separately. Thus, a 5-6-membered aryloxy(C₁₋₄ alkyl) is a 5-6 membered aryl ring, attached via an oxygen atom to a C₁₋₄ alkyl chain which, in turn, is attached to the rest of the molecule via the terminal carbon of the C₁₋₄ alkyl chain.

As used herein, the terms “halogen” or “halo” mean F, Cl, Br, or I.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example a C₁₋₃ haloalkyl could be —CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN or —CN.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. For example a C₁₋₃ cyanoalkyl could be —C(CN)₂CH₂CH₃ and a C₁₋₂ cyanoalkenyl could be ═CHC(CN)H₂.

As used herein, an “amino” group refers to —NH₂.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups. For example a C₁₋₃ aminoalkyl could be —CH(NH₂)CH₂CH₂NH₂ and a C₁₋₂ aminoalkoxy could be —OCH₂CH₂NH₂.

The term “hydroxyl” or “hydroxy” refers to —OH.

The terms “hydroxyalkyl”, “hydroxyalkenyl”, “hydroxyaliphatic”, and “hydroxyalkoxy” mean alkyl, alkenyl, aliphatic or alkoxy, as the case may be, substituted with one or more —OH groups. For example a C₁₋₃ hydroxyalkyl could be —CH₂(CH₂OH)CH₃ and a C₄ hydroxyalkoxy could be —OCH₂C(CH₃)(OH)CH₃.

As used herein, a “carbonyl”, used alone or in connection with another group refers to —C(O)— or —C(O)H. For example, as used herein, an “alkoxycarbonyl,” refers to a group such as —C(O)O(alkyl).

As used herein, an “oxo” refers to ═O, wherein oxo is usually, but not always, attached to a carbon atom. An aliphatic chain can be optionally interrupted by a carbonyl group or can optionally be substituted by an oxo group, and both expressions refer to the same: e.g. —CH₂—C(O)—CH₃.

As used herein, in the context of resin chemistry (e.g. using solid resins or soluble resins or beads), the term “linker” refers to a bifunctional chemical moiety attaching a compound to a solid support or soluble support.

In all other situations, a “linker”, as used herein, refers to a divalent group in which the two free valences are on different atoms (e.g. carbon or heteroatom) or are on the same atom but can be substituted by two different substituents. For example, a methylene group can be C₁ alkyl linker (—CH₂—) which can be substituted by two different groups, one for each of the free valences (e.g. as in Ph-CH₂-Ph, wherein methylene acts as a linker between two phenyl rings). Ethylene can be C₂ alkyl linker (—CH₂CH₂—) wherein the two free valences are on different atoms. The amide group, for example, can act as a linker when placed in an internal position of a chain (e.g. —CONH—). A linker can be the result of interrupting an aliphatic chain by certain functional groups or of replacing methylene units on said chain by said functional groups. E.g. a linker can be a C₁₋₆ aliphatic chain in which up to two methylene units are substituted by —C(O)— or —NH— (as in —CH₂—NH—CH₂—C(O)—CH₂— or —CH₂—NH—C(O)—CH₂—). An alternative way to define the same —CH₂—NH—CH₂—C(O)—CH₂— and —CH₂—NH—C(O)—CH₂— groups is as a C₃ alkyl chain optionally interrupted by up to two —C(O)— or —NH— moieties. Cyclic groups can also form linkers: e.g. a 1,6-cyclohexanediyl can be a linker between two R groups, as in

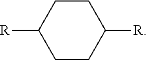

A linker can additionally be optionally substituted in any portion or position.

Divalent groups of the type R—CH═ or R₂C═, wherein both free valences are in the same atom and are attached to the same substituent, are also possible. In this case, they will be referred to by their IUPAC accepted names. For instance an alkylidene (such as, for example, a methylidene (═CH₂) or a ethylidene (═CH—CH₃)) would not be encompassed by the definition of a linker in this disclosure.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W. et al., “Protective Groups in Organic Synthesis”, Third Edition, John Wiley & Sons, New York: 1999, the entire contents of which is hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

As used herein, “amide coupling agent” or “amide coupling reagent” means a compound that reacts with the hydroxyl moiety of a carboxy moiety thereby rendering it susceptible to nucleophilic attack. Exemplary amide coupling agents include DIC (diisopropylcarbodiimide), EDCI (1-ethyl-3-(3-dimethylaminopropyl)carbodiimide), DCC (dicyclohexylcarbodiimide), BOP (benzotriazol-1-yloxy-tris(dimethylamino)-phosphonium hexafluorophosphate), pyBOP ((benzotriazol-1-yloxy)tripyrrolidinophosphonium hexafluorophosphate), etc.

In some of embodiments of Formula I, ring A is a 5 to 7-membered cycloaliphatic ring or a 5 or 6-membered non-aromatic heterocycle, wherein the 5 or 6-membered non-aromatic heterocycle contains from 1 to 3 heteroatoms independently selected from N, O or S, or alternatively the 5 or 6-membered non-aromatic heterocycle contains from 1 to 3 heteroatoms independently selected from O or S. In other embodiments, ring A is a 5 or 6-membered cycloaliphatic ring. In further embodiments, ring A is a 5-membered cycloaliphatic ring. In still further embodiments, ring A is a 6-membered cycloaliphatic ring. In yet further embodiments of Formula I, ring A is a 5 or 6-membered non-aromatic heterocycle.

In some embodiments of Formula I, ring A is a 6-membered non-aromatic heterocycle. In other embodiments, 1 or 2 ring atoms of the 6-membered non-aromatic heterocycle are selected from N or S, or alternatively 1 or 2 ring atoms of the 6-membered non-aromatic heterocycle are S heteroatoms. In further embodiments, ring A is a 6-membered non-aromatic heterocycle having one ring heteroatom, wherein the ring heteroatom is S or N. In still further embodiments, ring A is a 6-membered non-aromatic heterocycle having one sulfur ring heteroatom. In yet further embodiments, ring A is a 6-membered non-aromatic heterocycle having one nitrogen ring heteroatom.

In some embodiments of Formula I, ring A is a 5-membered non-aromatic heterocycle having one ring S heteroatom.

In some embodiments of Formula I, J^{A} is a substituent on a ring carbon atom and it is independently selected from halogen, C₁₋₆ aliphatic, oxo, —OR^{A}, —COR^{A}, —C(O)OR^{A}, —C(O)N(R^{A})₂, —CN, —N(R^{A})₂, —N(R^{A})C(O)R^{a}, —N(R^{A})C(O)OR^{a}, —SO₂R^{A}, —SO₂N(R^{A})₂ or —N(R^{A})SO₂N(R^{a})₂. In other embodiments, at least one J^{A} is a substituent on a ring carbon atom, and the at least one J^{A} is independently selected from halogen, C₁₋₆ aliphatic, oxo, —OR^{A}, —COR^{A}, —C(O)OR^{A}, —C(O)N(R^{A})₂, —CN, —N(R^{A})₂, —N(R^{A})C(O)R^{a}, —N(R^{A})C(O)OR^{a}, —SO₂R^{A}, —SO₂N(R^{A})₂ or —N(R^{A})SO₂N(R^{a})₂. In further embodiments, J^{A} is independently selected from halogen or a C₁₋₆ aliphatic group. In still further embodiments, J^{A} is a substituent on a ring carbon atom and independently selected from halogen. In yet further embodiments, J^{A} is independently selected from fluoro. In still further embodiments, J^{A} is a substituent on a ring carbon atom and independently selected from C₁₋₆ aliphatic groups. In yet further embodiments, J^{A} is methyl.

In some embodiments of Formula I, m is selected from 0, 1 or 2. In further embodiments, m is 1 or 2, and optionally J^{A} is independently selected from oxo or methyl. In other embodiments, m is 1. In yet other embodiments, m is 2. In still other embodiments, m is O.

In some embodiments of Formula I, ring A is a 5 or 6-membered non-aromatic heterocycle that contains at least one substituted ring nitrogen atom, wherein the at least one J^{A} on said nitrogen atom is a substituent independently selected from —C(O)R^{A}, —C(O)OR^{A}, —C(O)N(R^{A})₂, —SO₂R^{A}, —SO₂N(R^{A})₂, C₁₋₆ aliphatic, —(C₁₋₆ aliphatic)-R^{A}, a C₃₋₈ cycloaliphatic ring, a 6 or 10-membered aryl ring, a 4 to 8-membered heterocyclic ring or a 5 to 6-membered heteroaryl ring. In other embodiments, the at least one J^{A} is a substituent on the at least one ring nitrogen atom independently selected from —C(O)R^{A}, —C(O)N(R^{A})₂, —SO₂R^{A}, C₁₋₆ aliphatic, phenyl, a 5 or 6-membered heterocyclic ring or a 5 or 6-membered heteroaryl ring.

In some embodiments of Formula I, ring B is phenyl, a bicyclic 10-membered aryl ring, a 6-membered heteroaryl ring or a bicyclic 9 or 10-membered heteroaryl ring. In other embodiments, ring B is a 6-membered heteroaryl ring. In further embodiments, ring B is phenyl. In still further embodiments, ring B is substituted with 1 to 3 J^{B} substituents, wherein at least one of the J^{B} substituents is ortho to the attachment of L. In yet further embodiments, compounds of Formula I have phenyl or a 6-membered heteroaryl ring as ring B. In yet further embodiments, ring B is phenyl.

In some embodiments of Formula I, ring B is substituted with one J^{B} substituent ortho to the attachment of L. In other embodiments, compounds of Formula I have phenyl or a 6-membered heteroaryl ring as ring B and are substituted with one J^{B} substituent ortho to the attachment of L. In yet further embodiments, ring B is phenyl and it is substituted with one J^{B} substituent ortho to the attachment of L.

In some embodiments of Formula I, ring B is substituted with 1 to 3 J^{B} substituents and at least one of the J^{B} substituents is meta to the attachment of L. In other embodiments, compounds of Formula I have phenyl or a 6-membered heteroaryl ring as ring B and at least one of the J^{B} substituents is meta to the attachment of L. In further embodiments, ring B is phenyl and at least one of the J^{B} substituents is meta to the attachment of L.

In some embodiments of Formula I, ring B is substituted with one J^{B} substituent meta to the attachment of L. In other embodiments, compounds of Formula I have phenyl or a 6-membered heteroaryl ring as ring B and are substituted with one J^{B} substituent meta to the attachment of L. In some embodiments, ring B is phenyl and are substituted with one J^{B} substituent meta to the attachment of L.

In some embodiments of Formula I, at least one of the 1 to 3 J^{B} substituents is a substituent on a ring carbon atom independently selected from halogen, C₁₋₆ aliphatic, —CN, —N(R^{B})₂ and —OR^{B}. In other embodiments, at least one of the 1 to 3 J^{B} substituents is a substituent on a ring carbon atom independently selected from halogen, —OR^{B} and —CN. In further embodiments, at least one of the 1 to 3 J^{B} substituents is a substituent on a ring carbon atom independently selected from halogen atoms. In further embodiments, at least one of the 1 to 3 J^{B} substituents is a fluorine or chlorine atom attached to a ring carbon atom. In yet further embodiments, at least one of the 1 to 3 J^{B} substituents is a fluorine atom attached to a ring carbon atom.

In some embodiments of Formula I, there is one J^{B} substituent attached to ring B, the J^{B} substituent is ortho to the attachment of L and the J^{B} substituent is selected from halogen, C₁₋₆ aliphatic, —CN, —N(R^{B})₂ or —OR^{B}. Alternatively, the J^{B} substituent is selected from halogen, C₁₋₆ aliphatic or —CN. In some embodiments, the J^{B} substituent is halogen. In further embodiments, the J^{B} substituent is a chlorine or fluorine atom.

In some embodiments of Formula I, ring B is pyridinyl. In other embodiments, ring B is pyridin-3-yl. In further embodiments, ring B is pyrimidinyl. In still further embodiments, ring B is pyrimidin-5-yl.

In some embodiments of Formula I, ring D is pyridinyl, pyrimidinyl or 1,3,5-triazinyl. In other embodiments, ring D is pyridinyl or pyrimidinyl. In further embodiments, ring D is pyridinyl. In still further embodiments, ring D is pyridin-3-yl or pyridin-4-yl. In yet further embodiments, ring D is pyrimidinyl. In yet further embodiments, ring D is pyrimidin-5-yl or pyrimidin-2-yl.

In some embodiments of Formula I, J^{D} is a substituent on a ring carbon atom independently selected from halogen, an oxo group, —C(O)R^{D}, —CN, —N(R^{D})₂, —N═N—R^{D}, —N(R^{D})C(O)R^{d}, —N(R^{D})C(O)OR^{d}, —SO₂R^{D}, —SO₂N(R^{D})₂, —N(R^{D})SO₂R^{d}, a C₁₋₆ aliphatic, a —(C₁₋₆ aliphatic)-R^{D}, a 6 or 10-membered aryl ring, a 4 to 8-membered heterocyclic ring or a 5 to 6-membered heteroaryl ring, wherein each said 4 to 8-membered heterocylic ring and each said 5 to 6-membered heteroaryl ring contains between 1 and 3 heteroatoms independently selected from O, N and S; and wherein each said C₁₋₆ aliphatic, each said 6 or 10-membered aryl ring, each said 4 to 8-membered heterocyclic ring and each said 5 to 6-membered heteroaryl rings is independently substituted with from 0 to 3 instances of R⁵. In other embodiments of Formula I, J^{D} is independently selected from —N(R^{D})₂, —N═N—R^{D}, —N(R^{D})C(O)R^{d}, —N(R^{D})C(O)OR^{d}, a 6 or 10-membered aryl ring, a 4 to 8-membered heterocyclic ring or a 5 or 6-membered heteroaryl ring. In further embodiments, J^{D} is independently selected from —N(R^{D})₂, —N═N—R^{D}, —N(R^{D})C(O)R^{d}, —N(R^{D})C(O)OR^{d}, phenyl, a 5 or 6-membered heterocyclic ring or a 5 or 6-membered heteroaryl ring, wherein each said phenyl, each said 5 or 6-membered heterocyclic ring and each said 5 or 6-membered heteroaryl ring is independently substituted with from 0 to 3 instances of R⁵. In further embodiments, J^{D} is a substituent on a carbon ring atom independently selected from —N(R^{D})₂, —N(R^{D})C(O)R^{d} or —N(R^{D})C(O)OR^{d}. In still further embodiments, J^{D} is a substituent on a ring carbon atom independently selected from —N(R^{D})₂ groups. In yet further embodiments, J^{D} is —NH₂.

In some embodiments of Formula I, o is selected from 0, 1 or 2. In other embodiments, o is 0 or 1. In further embodiments, o is 1 and J^{D} is —NH₂.

In some of Formula I, o is 2 or 3, and at least one of the J^{D} substituents is —NH₂. In other embodiments, at least two J^{D} substituents are —NH₂.

In other embodiments of Formula I, ring A is a 5- or 6-membered cycloaliphatic, ring B is phenyl and ring D is pyrimidyl. In other embodiments, ring B is phenyl substituted with a halogen atom ortho or meta to the attachment of L, wherein the halogen is selected from chloro or fluoro. In further embodiments, ring B is phenyl substituted with a halogen atom ortho to the attachment of L, wherein the halogen is selected from chloro or fluoro. In still further embodiments, Ring D is pyrimidin-5-yl or pyrimidin-2-yl.

The invention also provides the compounds of Formula I excluding the compounds represented by CAS Registry Numbers RN 1017873-00-5, RN 1017873-82-3, RN 1017874-17-7, RN 150401-95-9 and RN 1025415-23-9, with the further proviso that the compounds of Formula I are not a derivatives or pharmaceutically acceptable salts of the compounds represented by CAS Registry Number RN 1017873-00-5, RN 1017873-82-3, RN 1017874-17-7, RN 150401-95-9 or RN 1025415-23-9, wherein a H atom of the compound represented by the CAS Registry Number is replaced with a methyl or ethyl group, or a methyl group of the compound represented by the CAS Registry Number is replaced with a H atom.

The compounds of the invention are defined herein by their chemical structures and/or chemical names. Where a compound is referred to by both a chemical structure and a chemical name, and the chemical structure and chemical name conflict, the chemical structure is determinative of the compound's identity.

In some embodiments, compounds of Formula I are selected from those listed in Table 1 herein. In other embodiments, compounds of Formula I are selected from Compound Nos. I-1 to I-37 and I-41 to I-49 listed in Table 1.

TABLE 1

| Compound | |
|---|---|
| No. | Chemical Structure |
| I-1 | 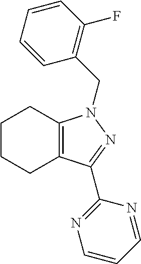 [SMILES: Fc1ccccc1Cn1nc(-c2ncccn2)c2c1CCCC2] |
| I-2 | 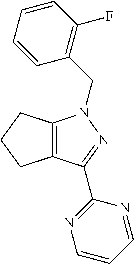 [SMILES: Fc1ccccc1Cn1nc(-c2ncccn2)c2c1CCC2] |
| I-3 | 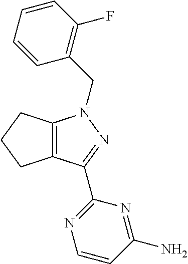 [SMILES: Nc1ccnc(-c2nn(Cc3ccccc3F)c3c2CCC3)n1] |
| I-4 | 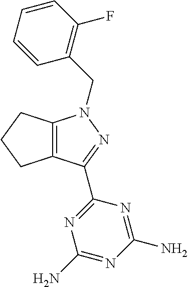 [SMILES: Nc1nc(N)nc(-c2nn(Cc3ccccc3F)c3c2CCC3)n1] |
| I-5 | 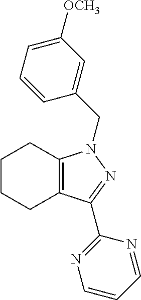 [SMILES: COc1cccc(Cn2nc(-c3ncccn3)c3c2CCCC3)c1] |
| I-6 | 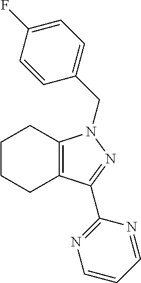 [SMILES: Fc1ccc(Cn2nc(-c3ncccn3)c3c2CCCC3)cc1] |
| I-7 | 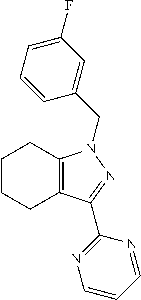 [SMILES: Fc1cccc(Cn2nc(-c3ncccn3)c3c2CCCC3)c1] |
| I-8 | 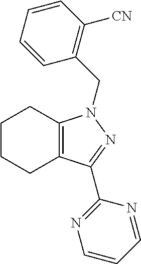 [SMILES: N#Cc1ccccc1Cn1nc(-c2ncccn2)c2c1CCCC2] |
| I-9 | 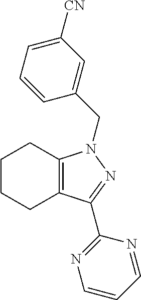 [SMILES: N#Cc1cccc(Cn2nc(-c3ncccn3)c3c2CCCC3)c1] |
| I-10 | 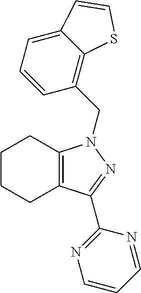 [SMILES: c1cnc(-c2nn(Cc3cccc4ccsc34)c3c2CCCC3)nc1] |
| I-11 | 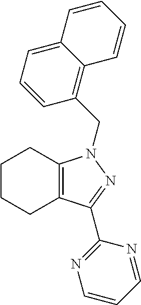 [SMILES: c1cnc(-c2nn(Cc3cccc4ccccc34)c3c2CCCC3)nc1] |
| I-12 | 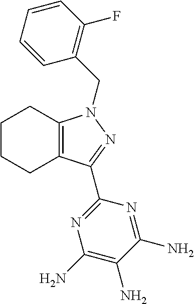 [SMILES: Nc1nc(-c2nn(Cc3ccccc3F)c3c2CCCC3)nc(N)c1N] |
| I-13 | 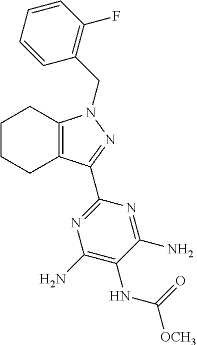 [SMILES: COC(=O)Nc1c(N)nc(-c2nn(Cc3ccccc3F)c3c2CCCC3)nc1N] |
| I-14 | 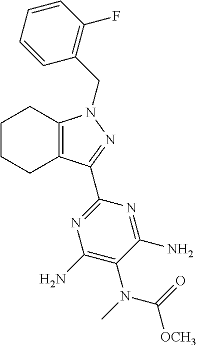 [SMILES: COC(=O)N(C)c1c(N)nc(-c2nn(Cc3ccccc3F)c3c2CCCC3)nc1N] |
| I-15 | 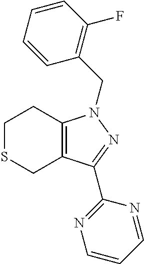 [SMILES: Fc1ccccc1Cn1nc(-c2ncccn2)c2c1CCSC2] |
| I-16 | 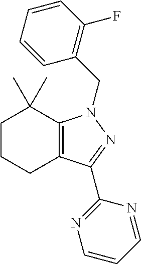 [SMILES: CC1(C)CCCc2c(-c3ncccn3)nn(Cc3ccccc3F)c21] |
| I-17 | 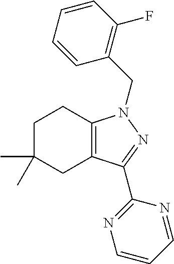 [SMILES: CC1(C)CCc2c(c(-c3ncccn3)nn2Cc2ccccc2F)C1] |
| I-18 | 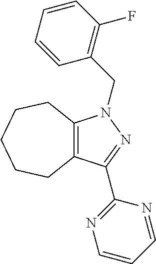 [SMILES: Fc1ccccc1Cn1nc(-c2ncccn2)c2c1CCCCC2] |
| I-19 | 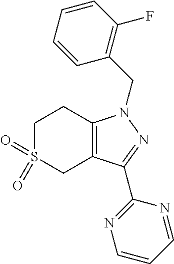 [SMILES: O=S1(=O)CCc2c(c(-c3ncccn3)nn2Cc2ccccc2F)C1] |
| I-20 | 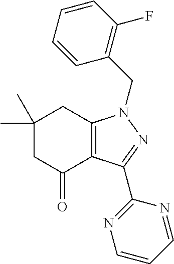 [SMILES: CC1(C)CC(=O)c2c(-c3ncccn3)nn(Cc3ccccc3F)c2C1] |
| I-21 | 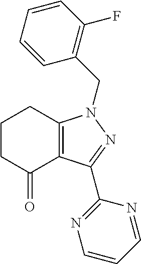 [SMILES: O=C1CCCc2c1c(-c1ncccn1)nn2Cc1ccccc1F] |
| I-22 | 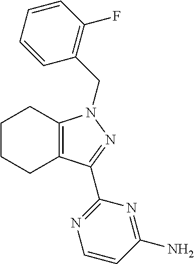 [SMILES: Nc1ccnc(-c2nn(Cc3ccccc3F)c3c2CCCC3)n1] |
| I-23 | 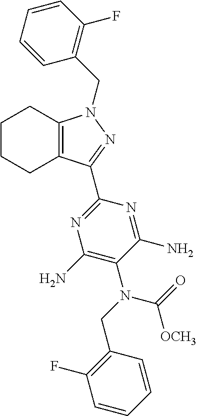 [SMILES: COC(=O)N(Cc1ccccc1F)c1c(N)nc(-c2nn(Cc3ccccc3F)c3c2CCCC3)nc1N] |
| I-24 | 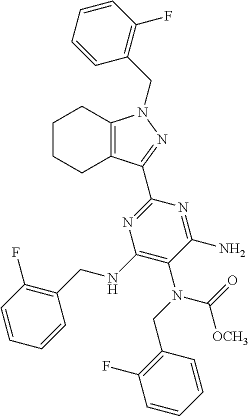 [SMILES: COC(=O)N(Cc1ccccc1F)c1c(N)nc(-c2nn(Cc3ccccc3F)c3c2CCCC3)nc1NCc1ccccc1F] |
| I-25 | 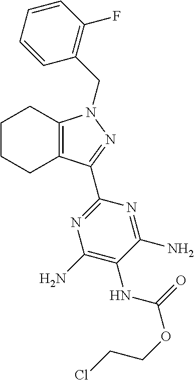 [SMILES: Nc1nc(-c2nn(Cc3ccccc3F)c3c2CCCC3)nc(N)c1NC(=O)OCCCl] |
| I-26 | 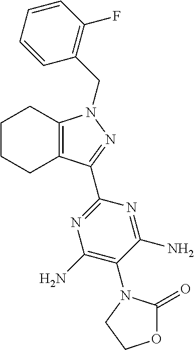 [SMILES: Nc1nc(-c2nn(Cc3ccccc3F)c3c2CCCC3)nc(N)c1N1CCOC1=O] |
| I-27 | 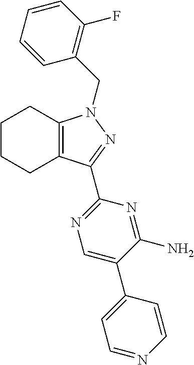 [SMILES: Nc1nc(-c2nn(Cc3ccccc3F)c3c2CCCC3)ncc1-c1ccncc1] |
| I-28 | 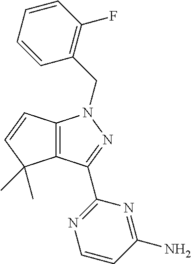 [SMILES: CC1(C)C=Cc2c1c(-c1nccc(N)n1)nn2Cc1ccccc1F] |
| I-29 | 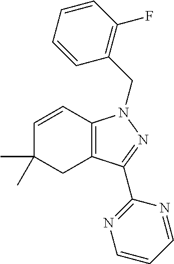 [SMILES: CC1(C)C=Cc2c(c(-c3ncccn3)nn2Cc2ccccc2F)C1] |
| I-30 | 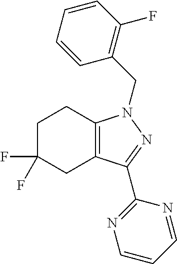 [SMILES: Fc1ccccc1Cn1nc(-c2ncccn2)c2c1CCC(F)(F)C2] |
| I-31 | 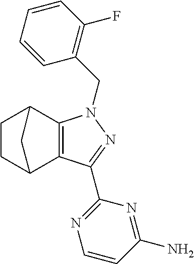 [SMILES: Nc1ccnc(-c2nn(Cc3ccccc3F)c3c2C2CCC3C2)n1] |
| I-32 | 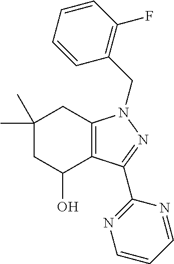 [SMILES: CC1(C)Cc2c(c(-c3ncccn3)nn2Cc2ccccc2F)C(O)C1] |
| I-33 | 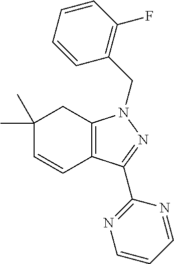 [SMILES: CC1(C)C=Cc2c(-c3ncccn3)nn(Cc3ccccc3F)c2C1] |
| I-34 | 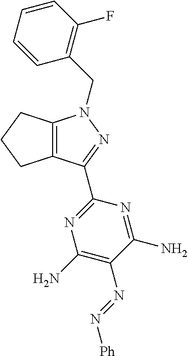 [SMILES: Nc1nc(-c2nn(Cc3ccccc3F)c3c2CCC3)nc(N)c1/N=N/c1ccccc1] |
| I-35 | 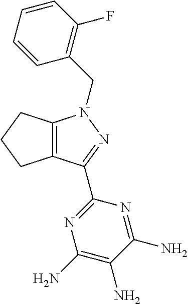 [SMILES: Nc1nc(-c2nn(Cc3ccccc3F)c3c2CCC3)nc(N)c1N] |
| I-36 | 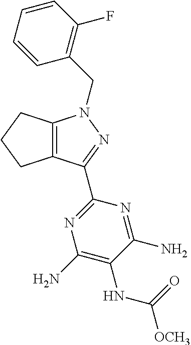 [SMILES: COC(=O)Nc1c(N)nc(-c2nn(Cc3ccccc3F)c3c2CCC3)nc1N] |
| I-37 | 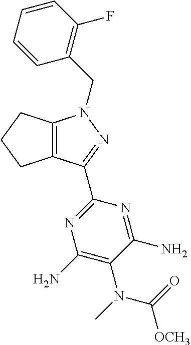 [SMILES: COC(=O)N(C)c1c(N)nc(-c2nn(Cc3ccccc3F)c3c2CCC3)nc1N] |
| I-38 | 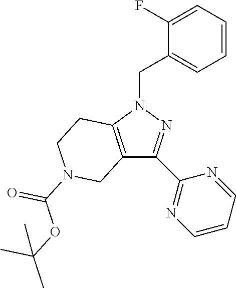 [SMILES: CC(C)(C)OC(=O)N1CCc2c(c(-c3ncccn3)nn2Cc2ccccc2F)C1] |
| I-39 | 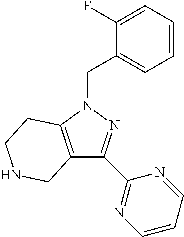 [SMILES: Fc1ccccc1Cn1nc(-c2ncccn2)c2c1CCNC2] |
| I-40 | 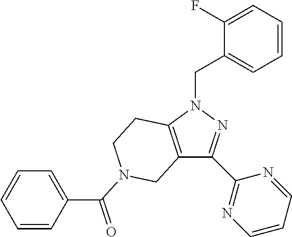 [SMILES: O=C(c1ccccc1)N1CCc2c(c(-c3ncccn3)nn2Cc2ccccc2F)C1] |
| I-41 | 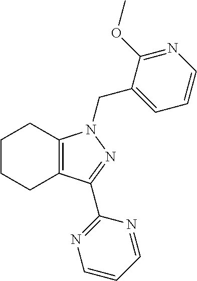 [SMILES: COc1ncccc1Cn1nc(-c2ncccn2)c2c1CCCC2] |
| I-42 | 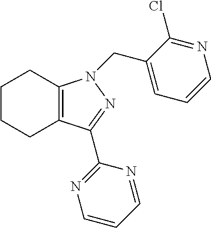 [SMILES: Clc1ncccc1Cn1nc(-c2ncccn2)c2c1CCCC2] |
| I-43 | 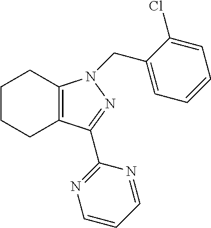 [SMILES: Clc1ccccc1Cn1nc(-c2ncccn2)c2c1CCCC2] |
| I-44 | 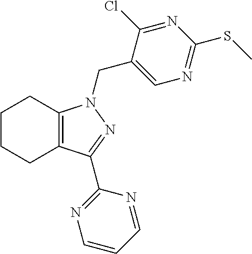 [SMILES: CSc1ncc(Cn2nc(-c3ncccn3)c3c2CCCC3)c(Cl)n1] |
| I-45 | 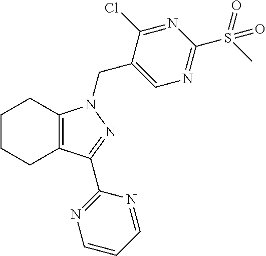 [SMILES: CS(=O)(=O)c1ncc(Cn2nc(-c3ncccn3)c3c2CCCC3)c(Cl)n1] |
| I-46 | 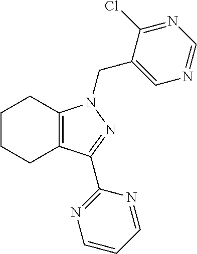 [SMILES: Clc1ncncc1Cn1nc(-c2ncccn2)c2c1CCCC2] |
| I-47 | 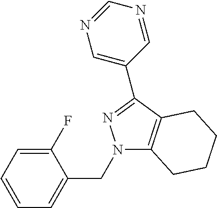 [SMILES: Fc1ccccc1Cn1nc(-c2cncnc2)c2c1CCCC2] |
| I-48 | 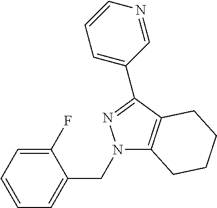 [SMILES: Fc1ccccc1Cn1nc(-c2cccnc2)c2c1CCCC2] |
| I-49 | 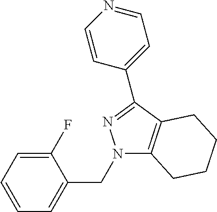 [SMILES: Fc1ccccc1Cn1nc(-c2ccncc2)c2c1CCCC2] |
| I-50 | 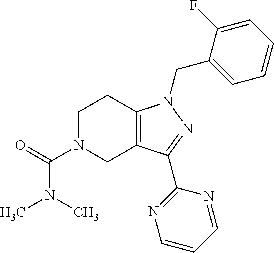 [SMILES: CN(C)C(=O)N1CCc2c(c(-c3ncccn3)nn2Cc2ccccc2F)C1] |
| I-51 | 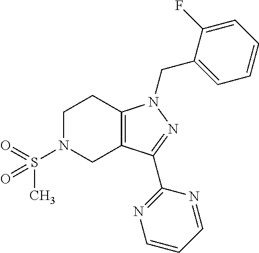 [SMILES: CS(=O)(=O)N1CCc2c(c(-c3ncccn3)nn2Cc2ccccc2F)C1] |
| I-52 | 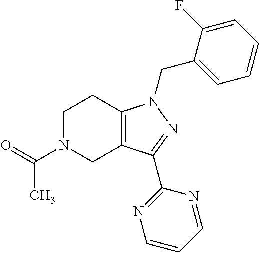 [SMILES: CC(=O)N1CCc2c(c(-c3ncccn3)nn2Cc2ccccc2F)C1] |
| I-53 | 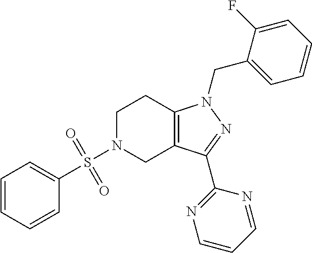 [SMILES: O=S(=O)(c1ccccc1)N1CCc2c(c(-c3ncccn3)nn2Cc2ccccc2F)C1] |
| I-54 | 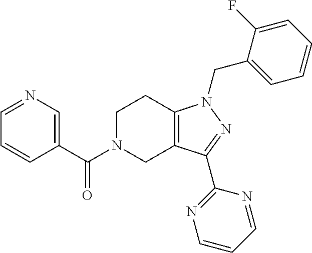 [SMILES: O=C(c1cccnc1)N1CCc2c(c(-c3ncccn3)nn2Cc2ccccc2F)C1] |
| I-55 | 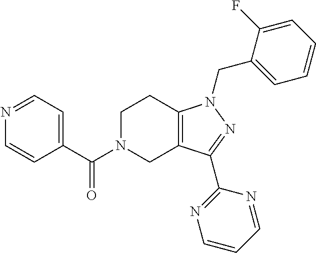 [SMILES: O=C(c1ccncc1)N1CCc2c(c(-c3ncccn3)nn2Cc2ccccc2F)C1] |
| I-56 | 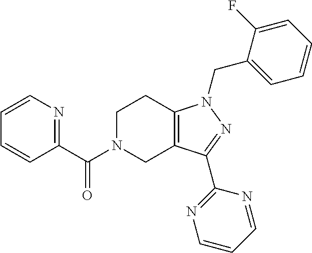 [SMILES: O=C(c1ccccn1)N1CCc2c(c(-c3ncccn3)nn2Cc2ccccc2F)C1] |
| I-57 | 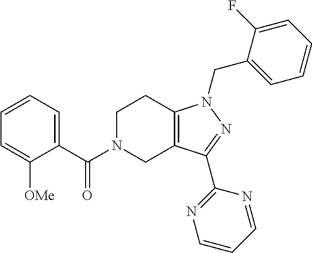 [SMILES: COc1ccccc1C(=O)N1CCc2c(c(-c3ncccn3)nn2Cc2ccccc2F)C1] |
| I-58 | 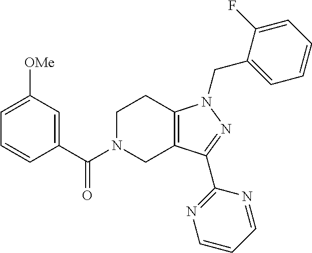 [SMILES: COc1cccc(C(=O)N2CCc3c(c(-c4ncccn4)nn3Cc3ccccc3F)C2)c1] |
| I-59 | 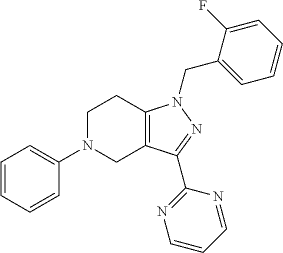 [SMILES: Fc1ccccc1Cn1nc(-c2ncccn2)c2c1CCN(c1ccccc1)C2] |
| I-60 | 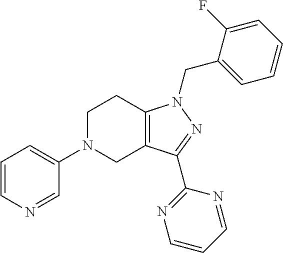 [SMILES: Fc1ccccc1Cn1nc(-c2ncccn2)c2c1CCN(c1cccnc1)C2] |
| I-61 | 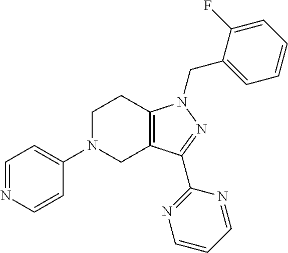 [SMILES: Fc1ccccc1Cn1nc(-c2ncccn2)c2c1CCN(c1ccncc1)C2] |
| I-62 | 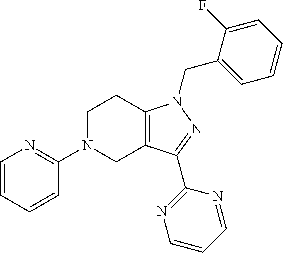 [SMILES: Fc1ccccc1Cn1nc(-c2ncccn2)c2c1CCN(c1ccccn1)C2] |
| I-63 | 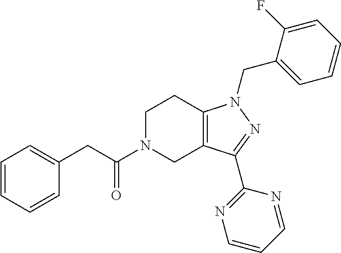 [SMILES: O=C(Cc1ccccc1)N1CCc2c(c(-c3ncccn3)nn2Cc2ccccc2F)C1] |

### Methods of Preparing the Compounds

The compounds of Formula I may be prepared according to the schemes and examples depicted and described below. Unless otherwise specified, the starting materials and various intermediates may be obtained from commercial sources, prepared from commercially available compounds or prepared using well-known synthetic methods. Another aspect of the present invention is a process for preparing the compounds of Formula I as disclosed herein.

General synthetic procedures for the compounds of this invention are described below. The synthetic schemes are presented as examples and do not limit the scope of the invention in any way.

### I. General Procedure A

The compounds of Formula I, wherein ring D is pyrimidine, can be prepared using General Procedure A depicted schematically below.

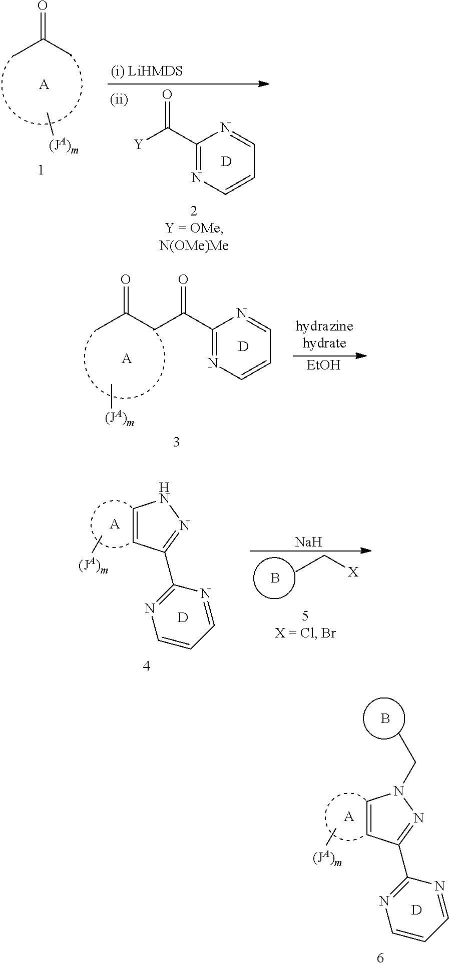

The General Procedure A can be separated into three main steps: dione formation, pyrazole formation and alkylation. In some of the embodiments, the three main steps can be carried out as disclosed below.

### Step 1: Dione formation

(lithium bis(trimethylsilyl)amide) LiHMDS is added to a cooled solution of ketone 1 in a nonpolar organic solvent such as tetrahydrofuran (THF). The reaction is allowed to warm to room temperature and stirred. The pyrimidine-derived electrophile 2 is added under stirring and the reaction proceeds under stirring until complete to provide the dione intermediate 3. Once complete, the reaction is quenched with NH₄Cl and an excess of dichlormethane (DCM) is added. The reaction mixture is separated into layers, and the aqueous portion is extracted with DCM. The organic portions are then combined, dried (e.g., with Na₂SO₄), filtered, and concentrated. The crude material is carried on to the pyrazole formation without any further purification.

### Step 2: Pyrazole formation

Dione 3 is dissolved in EtOH and treated with hydrazine hydrate. The reaction mixture is heated to reflux and stirred until cyclization is complete to form pyridine 4. Once complete, the reaction mixture is concentrated and carried on to the alkylation step without any further purification.

### Step 3: Alkylation

Pyrazole 4 is dissolved in a nonpolar organic solvent such as THF and cooled. NaH is added. The reaction mixture is allowed to warm to room temperature, and then stirred. Electrophile 5 is added under stirring and the reaction mixture is stirred at room temperature until the reaction is complete. Once complete, the reaction mixture is quenched with NH₄Cl and an excess of DCM is added. The reaction mixture is allowed to separate into layers, and the aqueous portion is extracted with DCM. The organic portions are then combined, dried (e.g., with Na₂SO₄), filtered, and concentrated. The crude oil is then purified, such as using SiO₂ chromatography and an appropriate gradient (e.g., ethyl acetate/hexanes or DCM/methanol), to obtain the desired product, compound 6.

### II. General Procedure B

Compounds of Formula I, wherein ring D is pyrimidine substituted with at least an amino group can be prepared with General Procedure B depicted schematically below.

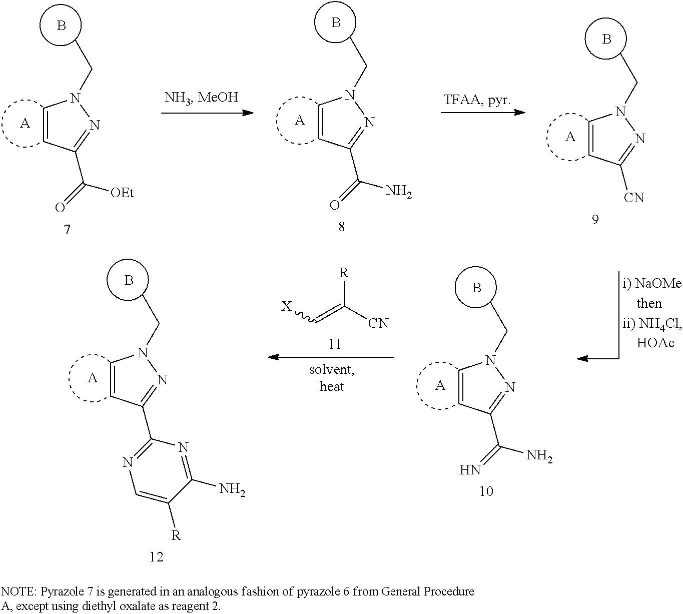

The General Procedure B can be separated into four main steps: primary amide formation, nitrile formation, carboximidamide formation and pyrimidine formation. In some of the embodiments, the four main steps can be carried out as disclosed below.

### Step 1: Primary Amide Formation

Ethyl ester 7 is mixed with an excess of a solution of ammonia in methanol and NaCN as a catalyst. The reaction mixture is then heated and stirred until the reaction is complete. Once complete, the reaction mixture is concentrated and the resulting material is diluted with DCM and filtered. The filtrate is concentrated and the crude oil is then purified using chromatograph, e.g., SiO₂ chromatography and an appropriate gradient (e.g., ethyl acetate/hexanes or DCM/methanol), to give amide 8, typically as a white foam.

### Step 2: Nitrite Formation

Amide 8 is dissolved in pyridine (0.25M) and cooled. Trifluoroacetic anhydride is then added. Once the reaction is complete, the reaction mixture is diluted with DCM and washed with water. The aqueous portion is back extracted with DCM. The organic portions are then combined, dried (e.g., with Na₂SO₄), filtered, and concentrated. The crude oil is then purified using chromatography such as SiO₂ chromatography and an appropriate gradient (e.g., ethyl acetate/hexanes or DCM/methanol) to give nitrile 9, typically as a white foam.

### Step 3: Carboximidamide Formation

The nitrile 9 is added to a solution of sodium methoxide in methanol. The reaction mixture is heated and stirred, e.g., for 3 hours. Acetic acid and ammonium chloride are added and the reaction is stirred at reflux, e.g., for 12-16 h. At this time, the reaction mixture is concentrated, and the remaining crude material is diluted with EtOAc and basified, e.g., by the addition of a saturated solution of sodium carbonate. The heterogeneous reaction mixture is allowed to separate into layers. The aqueous portion is then extracted with DCM. The organic portions are then combined, dried (e.g., with Na₂SO₄), filtered, and concentrated. The crude carboximidamide 10 is carried onto the cyclization reaction to generate the targeted pyrimidine.

### Step 4: Pyrimidine Formation

The carboximidamide 10 is dissolved in an appropriate solvent (e.g., xylene, toluene, or pyridine) and charged with vinyl nitrile 11. The reaction mixture is heated at reflux until >90% complete, e.g., as determined by LC/MS analysis. The reaction mixture is then concentrated, DCM is added, and the mixture is extracted with water. The aqueous portion is then extracted with DCM. The organic portions are then combined, dried (e.g., with Na₂SO₄), filtered, and concentrated. The crude oil is purified by preparative HPLC to give pyrimidine 12, as a (color) solid or liquid, etc.

### III. General Procedure C

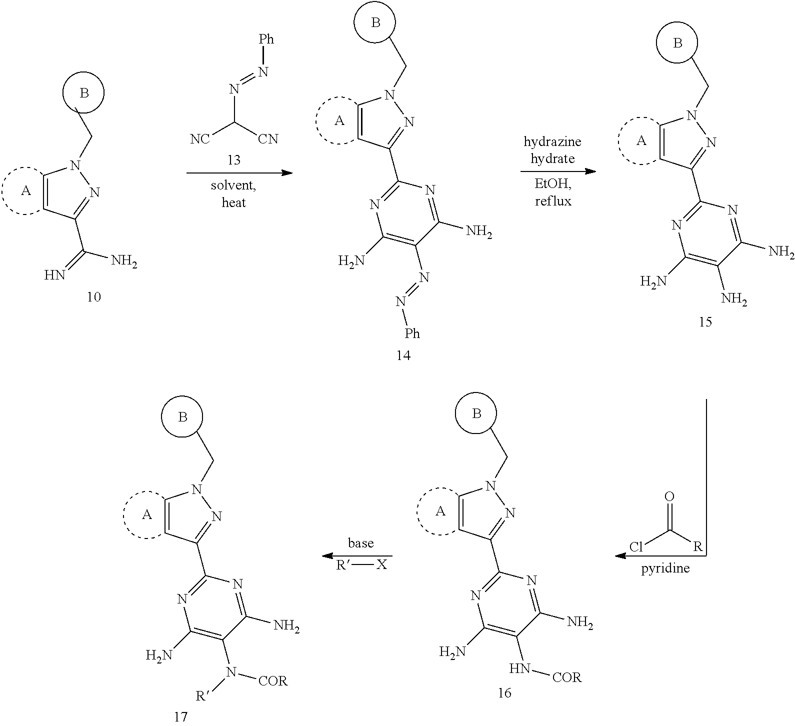

Some of the compounds of Formula I can be prepared using the General Procedure C, wherein ring D is pyrimidine substituted with at least an amino group. In some of the embodiments, the General Procedure C can be separated into four main steps: pyrimidine formation, hydrazinolysis, acylation and alkylation.

### Step 1: Pyrimidine Formation

Carboximidamide 10, optionally dissolved in toluene or DMF, is mixed with NaOMe. 2-(Phenyldiazenyl)malononitrile 13 is added, and the reaction mixture is heated until >90% complete, e.g., by LC/MS analysis. The reaction is then diluted with DCM and extracted with a concentrated aqueous solution of NH₄Cl. The aqueous portion is then extracted with DCM. The organic portion is dried (e.g., with Na₂SO₄), filtered, and concentrated. The crude oil is purified by chromatography, such as reverse phase, preparative HPLC or normal phase chromatography and a methanol/DCM gradient, to give the desired pyrimidine 14.

### Step 2: Hydrazinolysis

To a solution of pyrimidine 14, e.g., in EtOH, hydrazine hydrate is added. The reaction mixture is then heated to reflux and stirred until the reaction is complete. The crude reaction mixture is then concentrated and purified by chromatography, such as by reverse phase, preparative HPLC or by normal phase chromatography and a methanol/DCM gradient, to give the desired pyrimidine 15.

### Step 3: Acyclation

Tri-amino pyrimidine 15 is dissolved in pyridine and cooled, at which time the acylating reagent (acyl chloride, chloroformate, etc.) is added. The reaction mixture is stirred until the reaction is complete, e.g., by LC/MS analysis (typically taking more than 2 hours). The crude reaction mixture is then concentrated and purified by chromatography, e.g., by either reverse phase, preparative HPLC or by normal phase chromatography and a methanol/DCM gradient, to give the desired pyrimidine 16.

### Step 4: Alkylation

Pyrimidine 16 is dissolved in a solvent (most typically DMF) and cooled, e.g., to 0° C. Sodium hydride is added and then an electrophile is added (intramolecular variants do not require exogenous electrophiles). Once the reaction is complete, the reaction is quenched with water and extracted with DCM, for example, three times. The organic portion is then dried (e.g., with Na₂SO₄), filtered, and concentrated. The crude oil is purified by chromatography, such as by either reverse phase, preparative HPLC or by normal phase chromatography and a methanol/DCM gradient, to give the desired pyrimidine 17.

### IV. General Procedure D

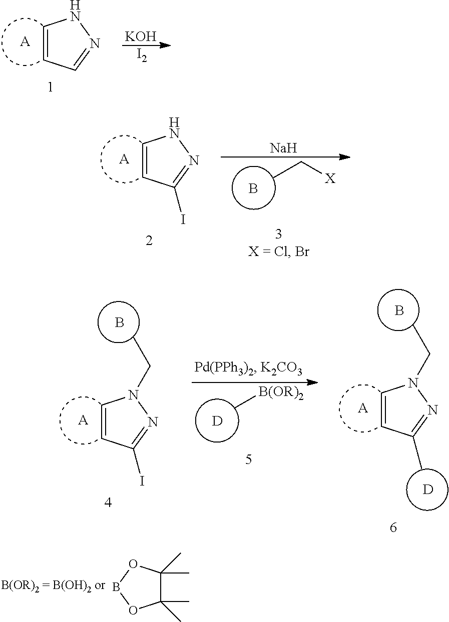

Some of the compounds of Formula I can be prepared using the General Procedure D as depicted schematically above. In some of the embodiments, the General Procedure D can be separated into three main steps: iodination, alkylation and cross coupling.

### Step 1: Iodination

Potassium hydroxide is mixed with pyrazole 1, e.g., with a solution of pyrazole 1 in DMF. The reaction mixture can be briefly sonicated to help dissolution. Iodine is then added and the reaction mixture is stirred until the reaction is complete (e.g., based on TLC and LC/MS analysis). Additional iodine could be added to drive the reaction to completion. Once completed, the reaction mixture is diluted with water and quenched with saturated sodium thiosulfate. The resulting crude mixture is extracted with EtOAc. The organic portions are then combined, washed three times with water and one time with brine, dried (e.g., with Na₂SO₄), filtered, and concentrated. The crude material is purified using chromatography such as SiO₂ chromatography and an appropriate gradient (e.g., ethyl acetate/hexanes or DCM/methanol) to give compound 2, as a solid or liquid.

### Step 2: Alkylation

To a solution of pyrazole 2 in THF is added NaH portion-wise. After stirring at room temperature, electrophile 3 is added and the reaction mixture is stirred at room temperature until completion, e.g., according to LC/MS analysis. Once completed, the reaction mixture is quenched with NH₄Cl, diluted with water. The crude mixture is extracted with EtOAc. The organic portion is dried (e.g., with Na₂SO₄), filtered, and concentrated. The crude oil is then purified using chromatography, e.g., with SiO₂ chromatography and an appropriate gradient (such as ethyl acetate/hexanes or DCM/methanol), to give compound 4, as a solid or liquid.

### Step 3: Cross Coupling

To a solid mixture of pyrazole 4, boronic acid or ester 5, potassium carbonate and tetrakis(triphenphenylphosphine)palladium(0) under a nitrogen atmosphere in a sealed tube is added DME/MeOH/DMF (e.g., at 2:3:1 ratio). The resulting suspension is heated at 120° C. until completion, e.g., according to LC/MS analysis. Once complete, the reaction mixture is diluted with EtOAc and filtered. The crude mixture is washed sequentially with 1N NaOH solution, water and brine, dried (e.g., with Na₂SO₄), filtered, and concentrated. The crude material is then purified using chromatography, e.g., SiO₂ chromatography and an appropriate gradient (such as ethyl acetate/hexanes or DCM/methanol), to give compound 6, as a solid or liquid.

### V. General Procedure E

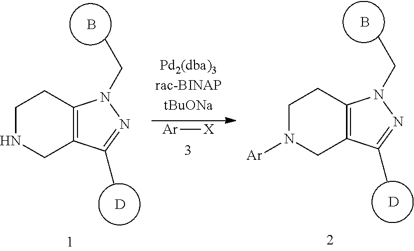

General procedure E can be used to prepare compounds of Formula I, wherein ring A is an azine and J^{A} is an aryl or heteroaryl ring. In the reaction scheme for General Procedure E shown above, Ar stands for the aryl or heteroaryl ring, X stands for halogen, wherein the halogen is Br or I, rac-BINAP stands for rac-2,2′-bis(diphenylphosphino)-1,1′-binaphthyl, and Pd₂(dba)₃ stands for tris(dibenzylideneacetone)dipalladium(0). Compound 2 is the compound of Formula I prepared by the General Procedure E. The General Procedure E involves a coupling reaction between compound 1 and the aryl or heteroaryl halide, Ar—X, i.e., compound 3.

Cross Coupling:

A nonpolar organic solvent such as toluene is added to a solid mixture of pyrazole 1, rac-2,2′-bis(diphenylphosphino)-1,1′-binaphthyl, tris(dibenzylideneacetone)dipalladium(0) and sodium tert-butoxide. Aryl halide 3, wherein the halide is a bromide or iodide, is added to the reaction mixture. The resulting suspension is heated, e.g., at 85° C. until the reaction is complete, e.g., according to LC/MS analysis. Once complete, the reaction mixture is mixed with an aqueous solution of an inorganic base such as a 1N NaOH solution and extracted with EtOAc. The organic portion is washed with brine, dried (e.g., with Na₂SO₄), filtered, and concentrated. The crude material is purified with chromatography such as SiO₂ chromatography and an appropriate gradient (e.g., ethyl acetate/hexanes or DCM/methanol) to give compound 2, as a target compound of Formula I.

### VI: General Procedure F

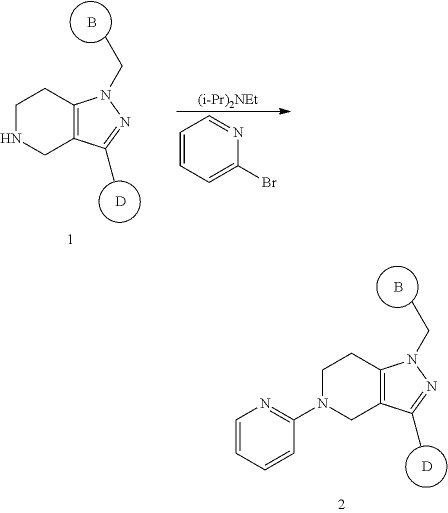

General procedure F can be used to prepare compounds of Formula I, wherein ring A is an azine and J^{A} is a pyridyl ring. In the reaction scheme for General Procedure F shown above, (i-Pr)₂NEt represents N-ethyl-N-isopropyl-2-propanamide.

Aromatic Substitution:

To a suspension of pyrazole 1 in 2-bromopyridine as solvent was added N-ethyl-N-isopropyl-2-propanamine. The reaction mixture was heated until the reaction is complete, e.g., according to TLC and LC/MS analysis. Once completed, the reaction was diluted with water and extracted with EtOAc. The organic portion is dried (e.g., with Na₂SO₄), filtered, and concentrated. The crude material is purified, e.g., using chromatography such as SiO₂ chromatography and an appropriate gradient (e.g., ethyl acetate/hexanes or DCM/methanol), to give compound 2, as a solid or liquid.

### Pharmaceutically Acceptable Salts, Co-Forms and Pro-Drugs of the Invention.

The phrase “pharmaceutically acceptable salt,” as used herein, refers to pharmaceutically acceptable organic or inorganic salts of a compound of Formula I. For use in medicine, the salts of the compounds of Formula I will be pharmaceutically acceptable salts. Other salts may, however, be useful in the preparation of the compounds of Formula I or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound of Formula I is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N.sup.1-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of Formula I is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated here by reference in its entirety.

In addition to the compounds described herein and their pharmaceutically acceptable salts, pharmaceutically acceptable solvates (e.g., hydrates) and co-crystals of these compounds and salts may also be employed in compositions to treat or prevent the herein identified disorders.

As used herein, the term “pharmaceutically acceptable solvate,” is a solvate formed from the association of one or more pharmaceutically acceptable solvent molecules to one of the compounds described herein. As used herein, the term “hydrate” means a compound described herein or a salt thereof that further includes a stoichiometric or non-stoichiometric amount of water bound by non-covalent intermolecular forces. The term solvate includes hydrates (e.g., hemihydrate, monohydrate, dihydrate, trihydrate, tetrahydrate, and the like).

“Pharmaceutically acceptable co-crystals” result when a pharmaceutically active compound crystallizes with another material (e.g. a carboxylic acid, a 4,4′-bipyridine or an excipient) that is also a solid at room temperature. Some pharmaceutically acceptable excipients are described in the next section. Other pharmaceutically acceptable substances that can be used to form co-crystals are exemplified by the GRAS (Generally regarded as safe) list of the US FDA.

In addition to the compounds described herein, pharmaceutically acceptable pro-drugs of these compounds may also be employed in compositions to treat or prevent the herein identified disorders.

A “pharmaceutically acceptable pro-drug” includes any pharmaceutically acceptable ester, salt of an ester or other derivative or salt thereof of a compound described herein which, upon administration to a recipient, is capable of providing, either directly or indirectly, a compound described herein. Particularly favoured pro-drugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species. The term “pro-drug” encompasses a derivative of a compound that can hydrolyze, oxidize, or otherwise react under biological conditions (in vitro or in vivo) to provide a compound described herein. Examples of pro-drugs include, but are not limited to, analogs or derivatives of compounds of Formula I that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of pro-drugs include derivatives of compounds that comprise —NO, —NO₂, —ONO, or —ONO₂ moieties. Pro-drugs can typically be prepared using well-known methods, such as those described by Burger's Medicinal Chemistry and Drug Discovery, (1995) 172-178, 949-982 (Manfred E. Wolff ed., 5th ed).

### Pharmaceutical Compositions and Methods of Administration.

The compounds herein disclosed, and their pharmaceutically acceptable salts, solvates, co-crystals and pro-drugs thereof may be formulated as pharmaceutical compositions or “formulations”.

A typical formulation is prepared by mixing a compound of Formula I, or a pharmaceutically acceptable salt, solvate, co-crystal or pro-drug thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of Formula I is being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., a compound of Formula I or pharmaceutical composition thereof) or aid in the manufacturing of the pharmaceutical product (i.e., medicament).

The formulations may be prepared using conventional dissolution and mixing procedures. For example, the bulk drug substance (i.e., compound of Formula I, a pharmaceutically acceptable salt, solvate, co-crystal or pro-drug thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8. When the agent described herein is a solid amorphous dispersion formed by a solvent process, additives may be added directly to the spray-drying solution when forming the mixture such as the additive is dissolved or suspended in the solution as a slurry which can then be spray dried. Alternatively, the additives may be added following spray-drying process to aid in the forming of the final formulated product.

The compound of Formula I or a pharmaceutically acceptable salt, solvate, co-crystal or pro-drug thereof is typically formulated into pharmaceutical dosage forms to provide an easily controllable dosage of the drug and to enable patient compliance with the prescribed regimen. Pharmaceutical formulations of compounds of Formula I, or a pharmaceutically acceptable salt, solvate, co-crystal or pro-drug thereof, may be prepared for various routes and types of administration. Various dosage forms may exist for the same compound, since different medical conditions may warrant different routes of administration.

The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. As a general proposition, the initial pharmaceutically effective amount of the inhibitor administered will be in the range of about 0.01-100 mg/kg per dose, namely about 0.1 to 20 mg/kg of patient body weight per day, with the typical initial range of compound used being 0.3 to 15 mg/kg/day.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically or pharmaceutically effective amount” of the compound to be administered will be governed by such considerations, and is the minimum amount necessary to ameliorate, cure or treat the disease or disorder or one or more of its symptoms.

The pharmaceutical compositions of Formula I will be formulated, dosed, and administered in a fashion, i.e., amounts, concentrations, schedules, course, vehicles, and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners, such as the age, weight, and response of the individual patient.

The term “prophylactically effective amount” refers to an amount effective in preventing or substantially lessening the chances of acquiring a disease or disorder or in reducing the severity of the disease or disorder before it is acquired or reducing the severity of one or more of its symptoms before the symptoms develop. Roughly, prophylactic measures are divided between primary prophylaxis (to prevent the development of a disease) and secondary prophylaxis (whereby the disease has already developed and the patient is protected against worsening of this process).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively; in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”. In general, one can provide for controlled release of the agents described herein through the use of a wide variety of polymeric carriers and controlled release systems including erodible and non-erodible matrices, osmotic control devices, various reservoir devices, enteric coatings and multiparticulate control devices.

“Sustained-release preparations” are the most common applications of controlled release. Suitable examples of sustained-release preparations include semipermeable matrices of solid hydrophobic polymers containing the compound, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent deaggregation to fine particules. This provides a larger surface area exposed to the dissolution medium, resulting in a faster dissolution rate.

Agents described herein can be incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use. One ingredient of this water-swollen matrix is the water-swellable, erodible, or soluble polymer, which may generally be described as an osmopolymer, hydrogel or water-swellable polymer. Such polymers may be linear, branched, or crosslinked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 daltons, for example, the Dow Methocel™ series E5, E15LV, E50LV and K100LY) and high viscosity (MW greater than 50,000 daltons, for example, E4MCR, E10MCR, K4M, K15M and K100M and the Methocel™ K series) HPMC. Other commercially available types of HPMC include the Shin Etsu Metolose 90SH series.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

Alternatively, the agents of the present invention may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g methyl acrylate-methyl methacrylate copolymers, polyvinyl chloride, polyethylene), hydrophilic polymers (e.g. ethyl cellulose, cellulose acetate, crosslinked polyvinylpyrrolidone (also known as crospovidone)), and fatty compounds (e.g. carnauba wax, microcrystalline wax, and triglycerides). Such devices are described further in Remington: The Science and Practice of Pharmacy, 20th edition (2000).

As noted above, the agents described herein may also be incorporated into an osmotic control device. Such devices generally include a core containing one or more agents as described herein and a water permeable, non-dissolving and non-eroding coating surrounding the core which controls the influx of water into the core from an aqueous environment of use so as to cause drug release by extrusion of some or all of the core to the environment of use. In certain embodiments, the coating is polymeric, aqueous-permeable, and has at least one delivery port. The core of the osmotic device optionally includes an osmotic agent which acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head). Nonlimiting examples of osmotic control devices are disclosed in U.S. patent application Ser. No. 09/495,061.

The amount of water-swellable hydrophilic polymers present in the core may range from about 5 to about 80 wt % (including for example, 10 to 50 wt %). Non limiting examples of core materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolat. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to thereby effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Nonlimiting examples of standard disintegrants include materials such as sodium starch glycolate (e.g., Explotab™ CLV), microcrystalline cellulose (e.g., Avicel™) microcrystalline silicified cellulose (e.g., ProSolv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 1-25% of the core agent.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described, for example, in EP378404.

When an agent described herein is a liquid or oil, such as a lipid vehicle formulation, for example as described in WO05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

As further noted above, the agents described herein may be provided in the form of microparticulates, generally ranging in size from about 10 μm to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art. Such multiparticulates may be made by any known process, such as wet- and dry-granulation processes, extrusion/spheronization, roller-compaction, melt-congealing, or by spray-coating seed cores. For example, in wet-and dry-granulation processes, the agent described herein and optional excipients may be granulated to form multiparticulates of the desired size.

The agents can be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology, New York: Marcel Dekker, 1992, volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The compounds described herein can be incorporated into pharmaceutically-acceptable nanoparticle, nanosphere, and nanocapsule formulations (Delie and Blanco-Prieto, 2005, Molecule 10:65-80). Nanocapsules can generally entrap compounds in a stable and reproducible way. To avoid side effects due to intracellular polymeric overloading, ultrafine particles (sized around 0.1 μm) can be designed using polymers able to be degraded in vivo (e.g. biodegradable polyalkyl-cyanoacrylate nanoparticles). Such particles are described in the prior art.

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g. using capsules, powders, solutions, suspensions, tablets, sublingual tablets and the like), by inhalation (e.g. with an aerosol, gas, inhaler, nebulizer or the like), to the ear (e.g. using ear drops), topically (e.g. using creams, gels, liniments, lotions, ointments, pastes, transdermal patches, etc), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

Formulations of a compound of Formula I that are suitable for oral administration may be prepared as discrete units such as tablets, pills, troches, lozenges, aqueous or oil suspensions, dispersible powders or granules, emulsions, hard or soft capsules, e.g. gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g. for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending the compound of Formula I in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds of Formula I contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds of Formula I may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds of Formula I include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a particle size for example in the range of 0.1 to 500 micros (including particles in a range between 0.1 and 500 microns in increments microns such as 0.5, 1, 30, 35 microns, etc) which is administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In another aspect, a compound of Formula I or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Therapeutic Methods

The present disclosure relates to stimulators of soluble guanylate cyclase (sGC), pharmaceutical formulations thereof and their use, alone or in combination with one or more additional agents, for treating and/or preventing various diseases, wherein an increase in the concentration of NO might be desirable, such as pulmonary hypertension, arterial hypertension, heart failure, atherosclerosis, inflammation, thrombosis, renal fibrosis and failure, liver cirrhosis, erectile dysfunction and other related cardiovascular disorders.

In one embodiment, the compounds herein disclosed are NO-independent, heme-dependent sGC stimulators that can be used to prevent and/or treat conditions, diseases or disorders in which it is considered desirable to increase the concentration of cGMP. Increased concentration of cGMP leads to vasodilation, inhibition of platelet aggregration and adhesion, anti-hypertensive effects, anti-remodelling effects, anti-apoptotic effects, anti-inflammatory effects and neuronal signal transmission effects. Thus, sGC stimulators may be used to treat and/or prevent a range of diseases and disorders, including but not limited to cardiovascular, endothelial, pulmonary, renal, hepatic and sexual diseases and disorders.

In other embodiments, the compounds here disclosed are sGC stimulators that may be useful in the prevention and/or treatment of diseases and disorders characterized by undesirable reduced bioavailability of and/or sensitivity to NO, such as those associated with conditions of oxidative stress or nitrosative stress.

Specific diseases of disorders which may be treated and/or prevented by administering an sGC stimulator, include but are not limited to: arterial hypertension, pulmonary hypertension, heart failure, stroke, septic shock, atherosclerosis, thrombosis, renal fibrosis, ischemic renal disease and renal failure, liver cirrhosis, erectile dysfunction, male and female sexual dysfunction, sickle cell anemia, asthma, chronic obstructive pulmonary disease, and neuroinflammatory diseases or disorders.

Pulmonary hypertension (PH) is a disease characterized by sustained elevations of blood pressure in the pulmonary vasculature (pulmonary artery, pulmonary vein and pulmonary capillaries), which results in right heart hypertrophy, eventually leading to right heart failure and death. Common symptoms of PH include shortness of breath, dizziness and fainting, all of which are exacerbated by exertion. Without treatment, median life expectancy following diagnosis is 2.8 years. PH exists in many different forms, which are categorized according to their aetiology. Categories include pulmonary arterial hypertension (PAH), PH with left heart disease, PH associated with lung diseases and/or hypoxaemia, PH due to chronic thrombotic and/or embolic disease and miscellaneous PH. PAH is rare in the general population, but the prevalence increases in association with certain common conditions such as HIV infection, scleroderma and sickle cell disease. Other forms of PH are generally more common than PAH, and, for instance, the association of PH with chronic obstructive pulmonary disease (COPD) is of particular concern. Current treatment for pulmonary hypertension depends on the stage and the mechanism of the disease.

The compounds according to Formula I of the present invention as well as pharmaceutically acceptable salts thereof, as stimulators of sGC, are useful in the prevention and/or treatment of the following types of diseases, conditions and disorders which can benefit from sGC stimulation:
(1) Peripheral or cardiac vascular disorders/conditions:
   pulmonary hypertension, pulmonary arterial hypertension, and associated pulmonary vascular remodeling (e.g. localized thrombosis and right heart hypertophy); pulmonary hypertonia; primary pulmonary hypertension, secondary pulmonary hypertension, familial pulmonary hypertension, sporadic pulmonary hypertension, pre-capillary pulmonary hypertension, idiopathic pulmonary hypertension, thrombotic pulmonary arteriopathy, plexogenic pulmonary arteriopathy; pulmonary hypertension associated with or related to: left ventricular dysfunction, hypoxemia, mitral valve disease, constrictive pericarditis, aortic stenosis, cardiomyopathy, mediastinal fibrosis, pulmonary fibrosis, anomalous pulmonary venous drainage, pulmonary venooclusive disease, pulmonary vasculitis, collagen vascular disease, congenital heart disease, pulmonary venous hypertension, interestitial lung disease, sleep-disordered breathing, apnea, alveolar hypoventilation disorders, chronic exposure to high altitude, neonatal lung disease, alveolar-capillary dysplasia, sickle cell disease, other coagulation disorders, chronic thromboemboli, pulmonary embolism (due to tumor, parasites or foreign material), connective tissue disease, lupus, schitosomiasis, sarcoidosis, chronic obstructive pulmonary disease, emphysema, chronic bronchitis, pulmonary capillary hemangiomatosis; histiocytosis X, lymphangiomatosis and compressed pulmonary vessels (such as due to adenopathy, tumor or fibrosing mediastinitis)
   disorders related to high blood pressure and decreased coronary blood flow such as increased acute and chronic coronary blood pressure, arterial hypertension and vascular disorder resulting from cardiac and renal complications (e.g. heart disease, stroke, cerebral ischemia, renal failure); congestive heart failure; thromboembolic disorders and ischemias such as myocardial infarction, stroke, transient ischemic attacks; stable or unstable angina pectoris; arrythmias; diastolic dysfunction; coronary insufficiency; atherosclerosis (e.g., associated with endothelial injury, platelet and monocyte adhesion and aggregation, smooth muscle proliferation and migration); restenosis (e.g. developed after thrombolysis therapies, percutaneous transluminal angioplasties (PTAs), percutaneous transluminal coronary angioplasties (PTCAs) and bypass); inflammation;
   liver cirrhosis, associated with chronic liver disease, hepatic fibrosis, hepatic stellate cell activation, hepatic fibrous collagen and total collagen accumulation; liver disease of necro-inflammatory and/or of immunological origin; and
(2) urogenital system disorders, such as renal fibrosis and renal failure resulting from chronic kidney diseases or insufficiently (e.g. due to accumulation/deposition and tissue injury, progressive sclerosis, glomerunephritis); prostate hypertrophy; erectile dysfunction; female sexual dysfunction and incontinence.

In some of the embodiments of the invention, the compounds according to Formula I as well as pharmaceutically acceptable salts thereof are also useful in the prevention and/or treatment of the following types of diseases, conditions and disorders which can benefit from sGC stimulation:
(a) a peripheral or cardiac vascular disorder or health condition selected from: pulmonary hypertension, pulmonary arterial hypertension, and associated pulmonary vascular remodeling, localized pulmonary thrombosis, right heart hypertophy, pulmonary hypertonia, primary pulmonary hypertension, secondary pulmonary hypertension, familial pulmonary hypertension, sporadic pulmonary hypertension, pre-capillary pulmonary hypertension, idiopathic pulmonary hypertension, thrombotic pulmonary arteriopathy, plexogenic pulmonary arteriopathy; pulmonary hypertension associated with or related to: left ventricular dysfunction, hypoxemia, mitral valve disease, constrictive pericarditis, aortic stenosis, cardiomyopathy, mediastinal fibrosis, pulmonary fibrosis, anomalous pulmonary venous drainage, pulmonary venooclusive disease, pulmonary vasculitis, collagen vascular disease, congenital heart disease, pulmonary venous hypertension, interestitial lung disease, sleep-disordered breathing, apnea, alveolar hypoventilation disorders, chronic exposure to high altitude, neonatal lung disease, alveolar-capillary dysplasia, sickle cell disease, other coagulation disorders, chronic thromboemboli, pulmonary embolism, connective tissue disease, lupus, schitosomiasis, sarcoidosis, chronic obstructive pulmonary disease, emphysema, chronic bronchitis, pulmonary capillary hemangiomatosis; histiocytosis X, lymphangiomatosis or compressed pulmonary vessels;
(b) liver cirrhosis, or
(c) a urogenital system disorder selected from renal fibrosis, renal failure resulting from chronic kidney diseases or insufficiently, erectile dysfunction or female sexual dysfunction.

In further embodiments of the invention, the compounds according to Formula I as well as pharmaceutically acceptable salts thereof are useful in the prevention and/or treatment of the following types of diseases, conditions and disorders which can benefit from sGC stimulation:
pulmonary hypertension, pulmonary arterial hypertension, and associated pulmonary vascular remodeling, localized pulmonary thrombosis, right heart hypertophy, pulmonary hypertonia, primary pulmonary hypertension, secondary pulmonary hypertension, familial pulmonary hypertension, sporadic pulmonary hypertension, pre-capillary pulmonary hypertension, idiopathic pulmonary hypertension, thrombotic pulmonary arteriopathy, plexogenic pulmonary arteriopathy or chronic obstructive pulmonary disease, liver cirrhosis, renal fibrosis, renal failure resulting from chronic kidney diseases or insufficiently, erectile dysfunction or female sexual dysfunction.

Alternatively, the compounds according to Formula I as well as pharmaceutically acceptable salts thereof are useful in the prevention and/or treatment of the following types of diseases, conditions and disorders which can benefit from sGC stimulation:
pulmonary hypertension, pulmonary arterial hypertension, and associated pulmonary vascular remodeling, pulmonary hypertonia, primary pulmonary hypertension, secondary pulmonary hypertension, familial pulmonary hypertension, sporadic pulmonary hypertension, pre-capillary pulmonary hypertension or idiopathic pulmonary hypertension.

The terms, “disease”, “disorder” and “condition” may be used interchangeably here to refer to a sGC, cGMP and/or NO mediated medical or pathological condition.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In some embodiments, the subject is a non-human animal such as a farm animal (e.g., a horse, cow, pig or sheep), or a pet (e.g., a dog, cat, guinea pig or rabbit). In some embodiments, the subject is a human.

The invention also provides a method for treating one of these diseases, conditions and disorders in a subject, comprising administering a therapeutically effective amount of the compound of Formula I, or a pharmaceutically acceptable salt thereof, in the subject in need of the treatment. Alternatively, the invention provides the use of the compound of Formula I, or a pharmaceutically acceptable salt thereof, in the treatment of one of these diseases, conditions and disorders in a subject in need of the treatment. The invention further provides a method of making a medicament useful for treating one of these diseases, conditions and disorders comprising using the compound of Formula I, or a pharmaceutically acceptable salt thereof.

The term “biological sample”, as used herein, refers to an in vitro or ex vivo sample, and includes, without limitation, cell cultures or extracts thereof; biopsied material obtained from a mammal or extracts thereof; blood, saliva, urine, feces, semen, tears, lymphatic fluid, ocular fluid, vitreous humour, or other body fluids or extracts thereof.

“Treat”, “treating” or “treatment” with regard to a disorder or disease refers to alleviating or abrogating the cause and/or the effects of the disorder or disease. As used herein, the terms “treat”, “treatment” and “treating” refer to the reduction or amelioration of the progression, severity and/or duration of a sGC, cGMP and/or NO mediated condition, or the amelioration of one or more symptoms (preferably, one or more discernable symptoms) of said condition (i.e. “managing” without “curing” the condition), resulting from the administration of one or more therapies (e.g., one or more therapeutic agents such as a compound or composition of the invention). In specific embodiments, the terms “treat”, “treatment” and “treating” refer to the amelioration of at least one measurable physical parameter of a sGC, cGMP and/or NO mediated condition. In other embodiments the terms “treat”, “treatment” and “treating” refer to the inhibition of the progression of a sGC, cGMP and/or NO mediated condition, either physically by, e.g., stabilization of a discernable symptom or physiologically by, e.g., stabilization of a physical parameter, or both. The term “preventing” as used herein refers to administering a medicament beforehand to avert or forestall the appearance of one or more symptoms of a disease or disorder. The person of ordinary skill in the medical art recognizes that the term “prevent” is not an absolute term. In the medical art it is understood to refer to the prophylactic administration of a drug to substantially diminish the likelihood or seriousness of a condition, or symptom of the condition and this is the sense intended in this disclosure. The Physician's Desk Reference, a standard text in the field, uses the term “prevent” hundreds of times. As used therein, the terms “prevent”, “preventing” and “prevention” with regard to a disorder or disease, refer to averting the cause, effects, symptoms or progression of a disease or disorder prior to the disease or disorder fully manifesting itself.

In one embodiment, the methods of the invention are a preventative or “pre-emptive” measure to a patient, specifically a human, having a predisposition (e.g. a genetic predisposition) to developing a sGC, cGMP and/or NO related disease, disorder or symptom.

In other embodiments, the methods of the invention are a preventative or “pre-emptive” measure to a patient, specifically a human, suffering from a disease, disorder or condition that makes him at risk of developing a sGC, cGM or NO related disease, disorder or symptom.

The compounds and pharmaceutical compositions described herein can be used alone or in combination therapy for the treatment or prevention of a disease or disorder mediated, regulated or influenced by sGC, cGMP and/or NO.

Compounds and compositions here disclosed are also useful for veterinary treatment of companion animals, exotic animals and farm animals, including, without limitation, dogs, cats, mice, rats, hamsters, gerbils, guinea pigs, rabbits, horses, pigs and cattle.

In other embodiments, the invention provides a method of stimulating sGC activity in a biological sample, comprising contacting said biological sample with a compound or composition of the invention. Use of a sGC stimulator in a biological sample is useful for a variety of purposes known to one of skill in the art. Examples of such purposes include, without limitation, biological assays and biological specimen storage.

### Combination Therapies

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When co-administered with other agents, e.g., when co-administered with another pain medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

When “combination therapy” is employed, an effective amount can be achieved using a first amount of a compound of Formula I or a pharmaceutically acceptable salt, solvate (e.g., hydrate), co-crystal or pro-drug thereof and a second amount of an additional suitable therapeutic agent.

In one embodiment of this invention, the compound of Formula I and the additional therapeutic agent are each administered in an effective amount (i.e., each in an amount which would be therapeutically effective if administered alone). In another embodiment, the compound of Structural Formula I and the additional therapeutic agent are each administered in an amount which alone does not provide a therapeutic effect (a sub-therapeutic dose). In yet another embodiment, the compound of Structural Formula I can be administered in an effective amount, while the additional therapeutic agent is administered in a sub-therapeutic dose. In still another embodiment, the compound of Structural Formula I can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When co-administration involves the separate administration of the first amount of a compound of Structural Formulae I and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of Formula I and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-cancer agent) to a subject.

Examples of other therapeutic agents that may be combined with a compound of this disclosure, either administered separately or in the same pharmaceutical composition, include, but are not limited to:
(1) Endothelium-derived releasing factor (EDRF);
(2) NO donors such as a nitrosothiol, a nitrite, a sydnonimine, a NONOate, a N-nitrosoamine, a N-hydroxyl nitrosamine, a nitrosimine, nitrotyrosine, a diazetine dioxide, an oxatriazole 5-imine, an oxime, a hydroxylamine, a N-hydroxyguanidine, a hydroxyurea or a furoxan. Some examples of these types of compounds include: glyceryl trinitrate (also known as GTN, nitroglycerin, nitroglycerine, and trinitrogylcerin), the nitrate ester of glycerol; sodium nitroprusside (SNP), wherein a molecule of nitric oxide is coordinated to iron metal forming a square bipyramidal complex; 3-morpholinosydnonimine (SIN-1), a zwitterionic compound formed by combination of a morpholine and a sydnonimine; S-nitroso-N-acetylpenicillamine (SNAP), an N-acetylated amino acid derivative with a nitrosothiol functional group; diethylenetriamine/NO (DETA/NO), a compound of nitric oxide covalently linked to diethylenetriamine; and NCX 4016, an m-nitroxymethyl phenyl ester of acetyl salicyclic acid. More specific examples of some of these classes of NO donors include: the classic nitrovasodilators, such as organic nitrate and nitrite esters, including nitroglycerin, amyl nitrite, isosorbide dinitrate, isosorbide 5-mononitrate, and nicorandil; Isosorbide (Dilatrate®-SR, Imdur®, Ismo®, Isordil®, Isordil®, Titradose®, Monoket®), FK 409 (NOR-3); FR 144420 (NOR-4); 3-morpholinosydnonimine; Linsidomine chlorohydrate (“SIN-1”); S-nitroso-N-acetylpenicillamine (“SNAP”); AZD3582 (ClNOD lead compound), NCX 4016, NCX 701, NCX 1022, HCT 1026, NCX 1015, NCX 950, NCX 1000, NCX 1020, AZD 4717, NCX 1510/NCX 1512, NCX 2216, and NCX 4040 (all available from NicOx S.A.), S-nitrosoglutathione (GSNO), S-nitrosoglutathione mono-ethyl-ester (GSNO-ester), 6-(2-hydroxy-1-methyl-nitrosohydrazino)-N-methyl-1-hexanamine (NOC-9) or diethylamine NONOate. Nitric oxide donors are also as disclosed in U.S. Pat. Nos. 5,155,137, 5,366,997, 5,405,919, 5,650,442, 5,700,830, 5,632,981, 6,290,981, 5,691,423 5,721,365, 5,714,511, 6,511,911, and 5,814,666, Chrysselis et al. (2002) J Med. Chem. 45:5406-9 (such as NO donors 14 and 17), and Nitric Oxide Donors for Pharmaceutical and Biological Research, Eds: Peng George Wang, Tingwei Bill Cai, Naoyuki Taniguchi, Wiley, 2005;
(3) Other substances that enhance cGMP concentrations such as protoporphyrin IX, arachidonic acid and phenyl hydrazine derivatives;
(4) Nitric Oxide Synthase substrates: for example, n-hydroxyguanidine based analogs, such as N[G]-hydroxy-L-arginine (NOHA), 1-(3,4-dimethoxy-2-chlorobenzylideneamino)-3-hydroxyguanidine, and PR5 (1-(3,4-dimethoxy-2-chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluormethyl) propylguanidine; and others reviewed in Cali et al. (2005, Current Topics in Medicinal Chemistry 5:721-736) and disclosed in the references cited therein;
(5) Compounds which enhance eNOS transcription: for example those described in WO 02/064146, WO 02/064545, WO 02/064546 and WO 02/064565, and corresponding patent documents such as US2003/0008915, US2003/0022935, US2003/0022939 and US2003/0055093. Other eNOS transcriptional enhancers including those described in US20050101599 (e.g. 2,2-difluorobenzo[1,3]dioxol-5-carboxylic acid indan-2-ylamide, and 4-fluoro-N-(indan-2-yl)-benzamide), and Sanofi-Aventis compounds AVE3085 and AVE9488 (CA Registry NO. 916514-70-0; Schafer et al., Journal of Thrombosis and Homeostasis 2005; Volume 3, Supplement 1: abstract number P1487);
(6) NO independent heme-independent sGC activators, including, but not limited to:
BAY 58-2667 (see patent publication DE19943635)

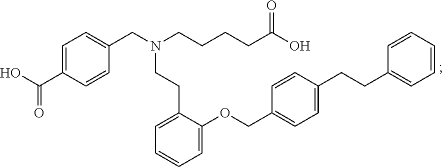 [SMILES: O=C(O)CCCCN(CCc1ccccc1OCc1ccc(CCc2ccccc2)cc1)Cc1ccc(C(=O)O)cc1]

HMR-1766 (ataciguat sodium, see patent publication WO2000002851)

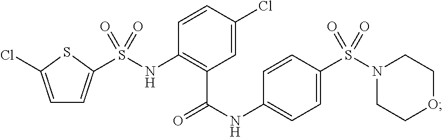 [SMILES: O=C(Nc1ccc(S(=O)(=O)N2CCOCC2)cc1)c1cc(Cl)ccc1NS(=O)(=O)c1ccc(Cl)s1]

S 3448 (2-(4-chloro-phenylsulfonylamino)-4,5-dimethoxy-N-(4-(thiomorpholine-4-sulfonyl)-phenyl)-benzamide (see patent publications DE19830430 and WO2000002851)

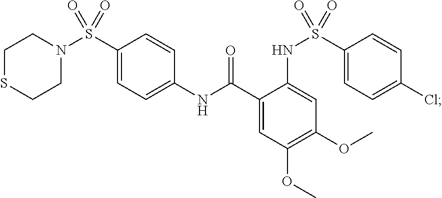 [SMILES: COc1cc(NS(=O)(=O)c2ccc(Cl)cc2)c(C(=O)Nc2ccc(S(=O)(=O)N3CCSCC3)cc2)cc1OC]

and
HMR-1069 (Sanofi-Aventis).
(7) Heme-dependent sGC stimulators including, but not limited to:
YC-1 (see patent publications EP667345 and DE19744026)

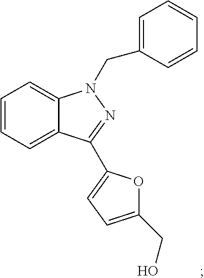 [SMILES: OCc1ccc(-c2nn(Cc3ccccc3)c3ccccc23)o1]

BAY 41-2272 (see patent publications DE19834047 and DE19942809)

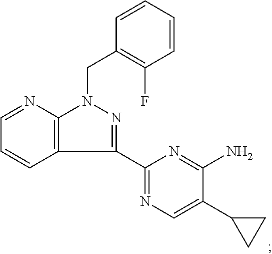 [SMILES: Nc1nc(-c2nn(Cc3ccccc3F)c3ncccc23)ncc1C1CC1]

BAY 41-8543 (see patent publication DE19834044)

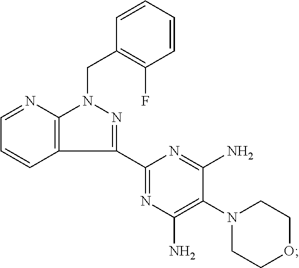 [SMILES: Nc1nc(-c2nn(Cc3ccccc3F)c3ncccc23)nc(N)c1N1CCOCC1]

BAY 63-2521 (see patent publication DE19834044)
CFM-1571 (see patent publication WO2000027394)

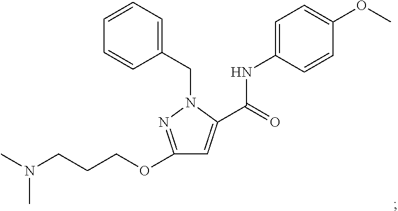

A350-619

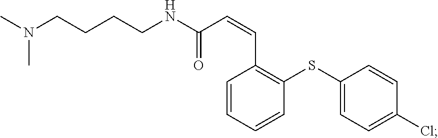 [SMILES: CN(C)CCCCNC(=O)/C=C\c1ccccc1Sc1ccc(Cl)cc1]

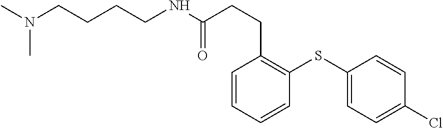 [SMILES: CN(C)CCCCNC(=O)CCc1ccccc1Sc1ccc(Cl)cc1]

A-344905;

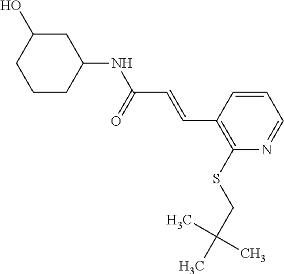 [SMILES: CC(C)(C)CSc1ncccc1/C=C/C(=O)NC1CCCC(O)C1]

A-778935;
and other compounds disclosed in Tetrahedron Letters (2003), 44(48): 8661-8663.
(8) Compounds that inhibit the degradation of cGMP, such as:
   PDE5 inhibitors, such as, for example, Sildenafil (Viagra®) and other related agents such as Avanafil, Lodenafil, Mirodenafil, Sildenafil citrate, Tadalafil (Clalis®), Vardenafil (Levitra®) and Udenafil; Alprostadil; and

### Dipyridamole;

(9) Calcium channel blockers such as:
   Dihydropyridine calcium channel blockers: Amlodipine (Norvasc), Aranidipine (Sapresta), Azelnidipine (Calblock), Barnidipine (HypoCa), Benidipine (Coniel), Cilnidipine (Atelec, Cinalong, Siscard), Clevidipine (Cleviprex), Efonidipine (Landel), Felodipine (Plendil), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas);
   Phenylalkylamine calcium channel blockers: Verapamil (Calan, Isoptin)

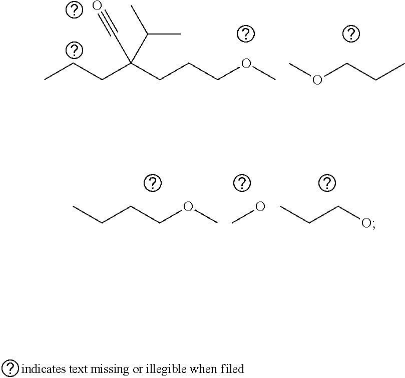

### Gallopamil (Procorum, D600);

### Benzothiazepines: Diltiazem (Cardizem);

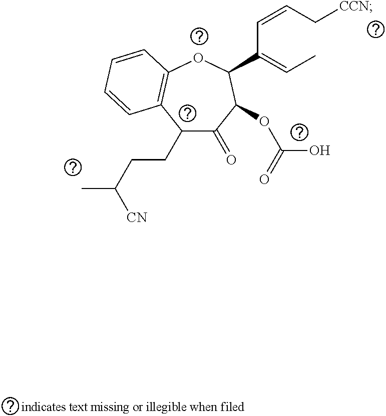

Nonselective calcium channel inhibitors such as: mibefradil, bepridil and fluspirilene, fendiline
(10) Endothelin receptor antagonists (ERAs): for instance the dual (ET_{A} and ET_{B}) endothelin receptor antagonist Bosentan (marketed as Tracleer®); Sitaxentan, marketed under the name Thelin®; Ambrisentan is marketed as Letairis® in U.S; dual/nonselective endothelin antagonist Actelion-1, that entered clinical trials in 2008;
(11) Prostacyclin derivatives: for instance prostacyclin (prostaglandin I₂), Epoprostenol (synthetic prostacyclin, marketed as Flolan®); Treprostinil (Remodulin®) Iloprost (Ilomedin®), Iloprost (marketed as Ventavis®); oral and inhaled forms of Remodulin® that are under development; Beraprost, an oral prostanoid available in Japan and South Korea;
(12) Antihyperlipidemics such as: cholestyramine, colestipol, and colesevelam; statins such as Atorvastatin, Simvastatin, Lovastatin and Pravastatin; Rosuvastatin; also combinations of statins, niacin, intestinal cholesterol absorption-inhibiting supplements (ezetimibe and others, and to a much lesser extent fibrates);
(13) Anticoagulants, such as the following types:
   Coumarines (Vitamin K antagonists): Warfarin® (Coumadin) mostly used in the US and UK; Acenocoumarol® and Phenprocoumon®, mainly used in other countries; Phenindione®;
   Heparin and derivative substances such as: Heparin; low molecular weight heparin, Fondaparinux and Idraparinux;
   Direct thrombin inhibitors such as: Argatroban, Lepirudin, Bivalirudin and Dabigatran; Ximelagatran (Exanta®), not approved in the US;
   Tissue plasminogen activators, used to dissolve clots and unblock arteries, such as Alteplase;
(14) Antiplatelet drugs: for instance thienopyridines such as Lopidogrel and Ticlopidine; Dipyridamole; Aspirin;
(15) ACE inhibitors, for example the following types:
   Sulfhydryl-containing agents such as Captopril (trade name Capoten®), the first ACE inhibitor and Zofenopril;
   Dicarboxylate-containing agents such as Enalapril (Vasotec/Renitec®); Ramipril (Altace/Tritace/Ramace/Ramiwin®); Quinapril (Accupril®) Perindopril (Coversyl/Aceon®); Lisinopril (Lisodur/Lopril/Novatec/Prinivil/Zestril®) and Benazepril (Lotensin®);
   Phosphonate-containing agents such as: Fosinopril;
   Naturally occurring ACE inhibitors such as: Casokinins and lactokinins, which are breakdown products of casein and whey that occur naturally after ingestion of milk products, especially cultured milk; The Lactotripeptides Val-Pro-Pro and Ile-Pro-Pro produced by the probiotic Lactobacillus helveticus or derived from casein also have ACE-inhibiting and antihypertensive functions;
(16) Supplemental oxygen therapy;
(17) Beta blockers, such as the following types:
   Non-selective agents: Alprenolol®, Bucindolol®, Carteolol®, Carvedilol® (has additional α-blocking activity), Labetalol® (has additional α-blocking activity), Nadolol®, Penbutolol® (has intrinsic sympathomimetic activity), Pindolol® (has intrinsic sympathomimetic activity), Propranolol® and Timolol®;
   β₁-Selective agents: Acebutolol® (has intrinsic sympathomimetic activity), Atenolol®, Betaxolol®, Bisoprolol®, Celiprolol®, Esmolol®, Metoprolol® and Nebivolol®;
   β₂-Selective agents: Butaxamine® (weak α-adrenergic agonist activity);
(18) Antiarrhythmic agents such as the following types:
   Type I (sodium channel blockers): Quinidine, Lidocaine, Phenyloin, Propafenone
   Type III (potassium channel blockers): Amiodarone, Dofetilide, Sotalol
   Type V: Adenosine, Digoxin
(19) Diuretics such as: Thiazide diuretics, e.g., chlorothiazide, chlorthalidone, and hydrochlorothiazide; Loop diuretics, such as furosemide; potassium-sparing diuretics such as amiloride, spironolactone, and triamterene; combinations of these agents;
(20) Exogenous vasodilators such as:
   Adenocard®, an adenosine agonist, primarily used as an anti-arrhythmic;
   Alpha blockers (which block the vasoconstricting effect of adrenaline);
   Atrial natriuretic peptide (ANP);
   Ethanol;
   Histamine-inducers, which complement proteins C3a, C4a and C5a work by triggering histamine release from mast cells and basophil granulocytes;
   Tetrahydrocannabinol (THC), major active chemical in marijuana which has minor vasodilatory effects;
   Papaverine, an alkaloid found in the opium poppy papaver somniferum;
(21) Bronchodilators: there are two major types of bronchodilator, β₂ agonists and anticholinergics, exemplified below:
   β₂ agonists: Salbutamol® or albuterol (common brand name: Ventolin) and Terbutaline® are short acting β₂ agonists for rapid relief of COPD symptoms. Long acting β₂ agonists (LABAs) such as Salmeterol® and Formoterol®;
   anticholinergics: Ipratropium® is the most widely prescribed short acting anticholinergic drug. Tiotropium® is the most commonly prescribed long-acting anticholinergic drug in COPD;
   Theophylline®, a broncodilator and phosphodiesterase inhibitor;
(22) Corticosteroids: such as beclomethasone, methylprednisolone, betamethasone, prednisone, prenisolone, triamcinolone, dexamethasone, fluticasone, flunisolide and hydrocortisone, and corticosteroid analogs such as budesonide
(23) Dietary supplements such as, for example: omega-3 oils; folid acid, niacin, zinc, copper, Korean red ginseng root, ginkgo, pine bark, Tribulus terrestris, arginine, Avena sativa, horny goat weed, maca root, muira puama, saw palmetto, and Swedish flower pollen; Vitamin C, Vitamin E, Vitamin K2; Testosterone supplements, Zoraxel, Naltrexone, Bremelanotide (formerly PT-141), Melanotan II, hMaxi-K; Prelox: a Proprietary mix/combination of naturally occurring ingredients, L-arginine aspartate and Pycnogenol;
(24) PGD2 receptor antagonists including, but not limited to, compounds described as having PGD2 antagonizing activity in United States Published Applications US20020022218, US20010051624, and US20030055077, PCT Published Applications WO9700853, WO9825919, WO03066046, WO03066047, WO03101961, WO03101981, WO04007451, WO0178697, WO04032848, WO03097042, WO03097598, WO03022814, WO03022813, and WO04058164, European Patent Applications EP945450 and EP944614, and those listed in: Torisu et al. 2004 Bioorg Med Chem Lett 14:4557, Torisu et al. 2004 Bioorg Med Chem Lett 2004 14:4891, and Torisu et al. 2004 Bioorg & Med Chem 2004 12:4685;
(25) Immunosuppressants such as cyclosporine (cyclosporine A, Sandimmune® Neoral®), tacrolimus (FK-506, Prograf®), rapamycin (sirolimus, Rapamune®) and other FK-506 type immunosuppressants, and mycophenolate, e.g., mycophenolate mofetil (CellCept®);
(26) Non-steroidal anti-asthmatics such as β2-agonists (e.g., terbutaline, metaproterenol, fenoterol, isoetharine, albuterol, salmeterol, bitolterol and pirbuterol) and β2-agonist-corticosteroid combinations (e.g., salmeterol-fluticasone (Advair®), formoterol-budesonid (Symbicort®)), theophylline, cromolyn, cromolyn sodium, nedocromil, atropine, ipratropium, ipratropium bromide, leukotriene biosynthesis inhibitors (zileuton, BAY1005);
(27) Non-steroidal antiinflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone);
(28) Cyclooxygenase-2 (COX-2) inhibitors such as celecoxib (Celebrex®), rofecoxib (Vioxx®), valdecoxib, etoricoxib, parecoxib and lumiracoxib; (opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine; and
(29) Anti-diabetic agents such as insulin and insulin mimetics, sulfonylureas (e.g., glyburide, meglinatide), biguanides, e.g., metformin (Glucophage®), α-glucosidase inhibitors (acarbose), thiazolidinone compounds, e.g., rosiglitazone (Avandia®), troglitazone (Rezulin®), ciglitazone, pioglitazone (Actos®) and englitazone.

### Kits

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for cotherapy use, a pharmaceutically acceptable carrier, vehicle or diluent. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

All references provided in the Examples are herein incorporated by reference. As used herein, all abbreviations, symbols and conventions are consistent with those used in the contemporary scientific literature. See, e.g. Janet S. Dodd, ed., The ACS Style Guide: A Manual for Authors and Editors, 2^{nd} Ed., Washington, D.C.: American Chemical Society, 1997, herein incorporated in its entirety by reference.

### Example I

### General Procedure A

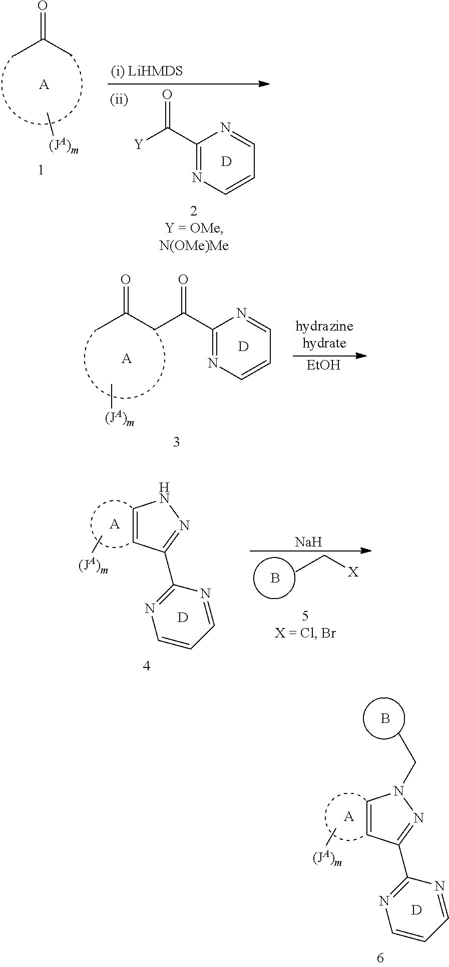

### Step 1: Dione Formation

To a cooled (0° C.) solution of ketone 1 in THF, was added LiHMDS (1.1 eq, 1.0 M in toluene). The reaction was allowed to warm to rt and stirred for 15 min. At this time, the pyrimidine-derived electrophile (2, 1.0 eq) was added and the reaction was stirred until complete (using TLC and LC/MS analysis) to provide 3. Once complete, the reaction was quenched with NH₄Cl and transferred to a separatory funnel using an excess of DCM. The layers were separated, and the aqueous portion was extracted an additional two times with DCM. The organic portions were then combined, dried (Na₂SO₄), filtered, and concentrated. The crude material was carried on to pyrazole formation without any further purification.

### Step 2: Pyrazole Formation

Dione 3 was dissolved in EtOH (0.05-0.1M) and treated with hydrazine hydrate (1-3 eq). Reaction was heated to reflux and stirred until cyclization was complete (by LC/MS analysis) to pyridine 4. Once complete, reaction was directly concentrated and carried on to the alkylation step without any further purification.

### Step 3: Alkylation

Pyrazole 4 was dissolved in THF and cooled to 0° C. NaH (1.1 eq, 60% in dispersion oil) was added (bubbling), the reaction was warmed to rt, and then stirred for 10 min. At this time, electrophile 5 (1.5 eq) was added and the reaction was stirred at rt until complete by LC/MS analysis. Once complete, the reaction was quenched with NH₄Cl and transferred to a separatory funnel using an excess of DCM. The layers were separated, and the aqueous portion was extracted an additional two times with DCM. The organic portions were then combined, dried (Na₂SO₄), filtered, and concentrated. The crude oil was then purified using SiO₂ chromatography and an appropriate gradient (ethyl acetate/hexanes or DCM/methanol) to give compound 6 (color and physical state below).
The following compounds were synthesized following General Procedure A using the appropriate ketone 1 in step 1 and electrophile 5 in step 3:

Compound I-1 was synthesized as a white solid (10% yield over 3 steps) following using cyclohexanone (acetone 1) in step 1 and 2-fluorobenzyl bromide (electrophile 5) in step 3.

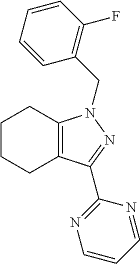 [SMILES: Fc1ccccc1Cn1nc(-c2ncccn2)c2c1CCCC2]

¹H NMR (400 MHz, CDCl₃) δ 8.79 (d, 2H), 7.25-7.20 (m, 1H), 7.13 (t, 1H), 7.07-6.96 (m, 3H), 5.44 (s, 2H), 2.94 (t, 2H), 2.50 (t, 2H), 1.83-1.73 (m, 4H) ppm.

Compound I-2 was synthesized as a white solid (1% yield over 3 steps) using cyclopentanone in step 1 and 2-fluorobenzyl bromide in step 3.

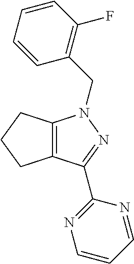 [SMILES: Fc1ccccc1Cn1nc(-c2ncccn2)c2c1CCC2]

¹H NMR (400 MHz, CDCl₃) δ 8.78 (d, 2H), 7.30-7.20 (m, 2H), 7.13 (t, 1H), 7.11-7.05 (m, 2H), 5.41 (s, 2H), 2.95-2.91 (m, 2H), 2.57-2.54 (m, 4H) ppm.

Compound I-5 was synthesized as an off-white solid (8% yield over 3 steps) following General Procedure A using cyclohexanone in step 1 and 3-methoxybenzyl bromide in step 3.

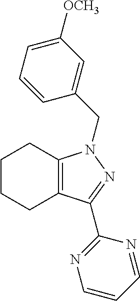 [SMILES: COc1cccc(Cn2nc(-c3ncccn3)c3c2CCCC3)c1]

¹H NMR (400 MHz, CDCl₃) δ 8.78 (d, 2H), 7.20 (t, 1H), 7.11 (t, 1H), 6.79-6.71 (m, 3H), 5.35 (s, 2H), 3.74 (s, 3H), 2.92 (t, 2H), 2.45 (t, 2H), 1.80-1.72 (m, 4H) ppm.

Compound I-6 was synthesized as an off-white solid (2% yield over 3 steps) using cyclohexanone in step 1 and 4-fluorobenzyl bromide in step 3.

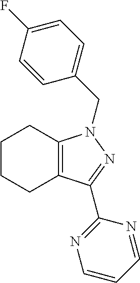 [SMILES: Fc1ccc(Cn2nc(-c3ncccn3)c3c2CCCC3)cc1]

¹H NMR (400 MHz, CDCl₃) δ 8.79 (d, 2H), 7.17-7.12 (m, 3H), 7.00-6.96 (m, 1H), 6.98 (t, 1H), 5.30 (s, 2H), 2.93 (t, 2H), 2.45 (t, 2H), 1.80-1.73 (m, 4H) ppm.

Compound I-7 was synthesized as an off-white solid (5% yield over 3 steps) using cyclohexanone in step 1 and 3-fluorobenzyl bromide in step 3.

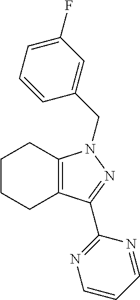 [SMILES: Fc1cccc(Cn2nc(-c3ncccn3)c3c2CCCC3)c1]

¹H NMR (400 MHz, CDCl₃) δ 8.79 (d, 2H), 7.29-7.23 (m, 1H), 7.14 (t, 1H), 6.96-6.92 (m, 2H), 6.86-6.83 (m, 1H), 5.37 (s, 2H), 2.95 (t, 2H), 2.46 (t, 2H), 1.82-1.74 (m, 4H) ppm.

Compound I-8 was synthesized as an off-white solid (3% yield over 3 steps) following General Procedure A using cyclohexanone in step 1 and 2-cyanobenzyl bromide in step 3.

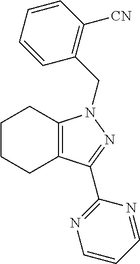 [SMILES: N#Cc1ccccc1Cn1nc(-c2ncccn2)c2c1CCCC2]

¹H NMR (400 MHz, CDCl₃) δ 8.80 (d, 2H), 7.67 (dd, 1H), 7.49 (ddd, 1H), 7.36 (ddd, 1H), 7.16 (t, 1H), 7.05 (dd, 1H), 5.60 (s, 2H), 2.96 (t, 2H), 2.50 (t, 2H), 1.84-1.75 (m, 4H) ppm.

Compound I-9 was synthesized as an off-white solid (5% yield over 3 steps) using cyclohexanone in step 1 and 3-cyanobenzyl bromide in step 3.

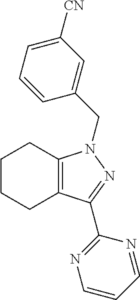 [SMILES: N#Cc1cccc(Cn2nc(-c3ncccn3)c3c2CCCC3)c1]

¹H NMR (400 MHz, CDCl₃) δ 8.80 (d, 2H), 7.56-7.54 (m, 1H), 7.42-7.38 (m, 3H), 7.15 (t, 1H), 5.40 (s, 2H), 2.95 (t, 2H), 2.46 (t, 2H), 1.84-1.74 (m, 4H) ppm.

Compound I-10 was synthesized as an off-white solid (5% yield over 3 steps) using cyclohexanone in step 1 and 7-(bromomethyl)benzothiophene in step 3.

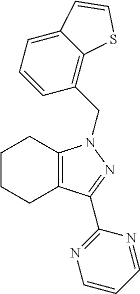 [SMILES: c1cnc(-c2nn(Cc3cccc4ccsc34)c3c2CCCC3)nc1]

¹H NMR (400 MHz, CDCl₃) δ 8.80 (d, 2H), 7.74 (dd, 1H), 7.44 (dd, 1H), 7.38 (d, 1H), 7.29 (t, 1H), 7.13 (t, 1H), 6.92 (d, 1H), 5.62 (s, 2H), 2.98-2.95 (m, 2H), 2.41-2.38 (m, 2H), 1.78-1.71 (m, 4H) ppm.

Compound I-11 was synthesized as an off-white solid (5% yield over 3 steps) using cyclohexanone in step 1 and 1-(chloromethyl)naphthalene in step 3.

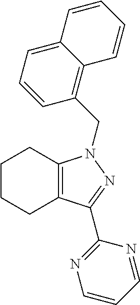 [SMILES: c1cnc(-c2nn(Cc3cccc4ccccc34)c3c2CCCC3)nc1]

¹H NMR (400 MHz, CDCl₃) δ 8.80 (d, 2H), 8.10 (dd, 1H), 7.89-7.86 (m, 1H), 7.77 (d, 1H), 7.57-7.49 (m, 2H), 7.35 (dd, 1H), 7.14 (t, 1H), 6.88 (dd, 1H), 5.88 (s, 2H), 2.99-2.70 (m, 2H), 2.39-2.37 (m, 2H), 1.76-1.71 (m, 4H) ppm.

Compound I-16 was synthesized as a yellow, viscous oil (7% yield over 3 steps) using 2,2-dimethylcyclohexanone in step 1 and 2-fluorobenzyl bromide in step 3.

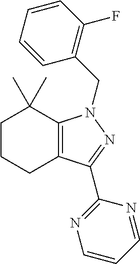 [SMILES: CC1(C)CCCc2c(-c3ncccn3)nn(Cc3ccccc3F)c21]

¹H NMR (400 MHz, CDCl₃) δ 8.78 (d, 2H), 7.20-7.15 (m, 1H), 7.13 (t, 1H), 7.03-6.98 (m, 1H), 6.76 (ddd, 1H), 6.70-6.66 (m, 1H), 5.63 (s, 2H), 2.74 (t, 2H), 1.81-1.75 (m, 2H), 1.66-1.63 (m, 2H), 1.20 (s, 6H) ppm.

Compound I-17 was synthesized as a white solid (17% yield over 3 steps) using 4,4-dimethylcyclohexanone in step 1 and 2-fluorobenzyl bromide in step 3.

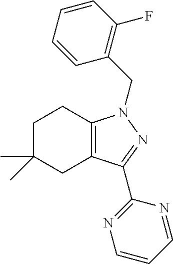 [SMILES: CC1(C)CCc2c(c(-c3ncccn3)nn2Cc2ccccc2F)C1]

¹H NMR (400 MHz, CDCl₃) δ 8.78 (d, 2H), 7.24-7.18 (m, 1H), 7.11 (t, 1H), 7.05-6.98 (m, 2H), 6.91 (ddd, 1H), 5.44 (s, 2H), 2.74 (s, 2H), 2.47 (t, 2H), 1.55 (t, 2H), 0.98 (s, 6H) ppm.

Compound I-18 was synthesized as a white solid (18% yield over 3 steps) using cycloheptanone in step 1 and 2-fluorobenzyl bromide in step 3.

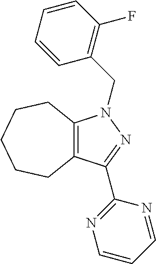 [SMILES: Fc1ccccc1Cn1nc(-c2ncccn2)c2c1CCCCC2]

¹H NMR (400 MHz, CDCl₃) δ 8.80 (d, 2H), 7.26-7.19 (m, 1H), 7.15 (t, 1H), 7.06-7.01 (m, 2H), 6.87-6.83 (m, 1H), 5.50 (s, 2H), 3.20-3.17 (m, 2H), 2.65-2.62 (m, 2H), 1.84-1.79 (m, 2H), 1.71-1.66 (m, 2H), 1.63-1.57 (m, 2H) ppm.

Compound I-20 was synthesized as a white solid (10% yield over 3 steps) using 5,5-dimethylcyclohexane-1,3-dione in step 1 and 2-fluorobenzyl bromide in step 3. [NOTE: In the first step, the electrophile was formed in situ from CDI (1.05 eq) and pyrimidine-2-carboxylic acid (1.0 eq) in CHCl₃ at 40° C. for 2 h, then was directly subjected to 5,5-dimethylcyclohexane-1,3-dione (1.0 eq) and DMAP (1.0 eq) for 14 h at 80° C. to form desired adduct.]

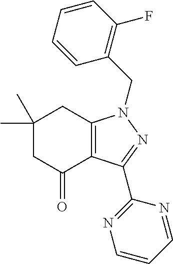 [SMILES: CC1(C)CC(=O)c2c(-c3ncccn3)nn(Cc3ccccc3F)c2C1]

¹H NMR (400 MHz, CDCl₃) δ 8.90 (d, 2H), 7.32-7.29 (m, 1H), 7.30 (t, 1H), 7.12-7.07 (m, 3H), 5.46 (s, 2H), 2.69 (s, 2H), 2.45 (s, 2H), 1.12 (s, 6H) ppm.

Compound I-32 was synthesized as an off white solid (80%) via reduction of Compound I-20 with sodium borohydride (1 eq, solvent=EtOH, time=14 h).

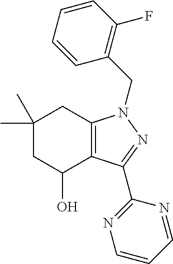 [SMILES: CC1(C)Cc2c(c(-c3ncccn3)nn2Cc2ccccc2F)C(O)C1]

¹H NMR (400 MHz, CDCl₃) δ 8.83 (d, 2H), 7.27-7.22 (m, 2H), 7.08-7.00 (m, 2H), 6.95-6.91 (m, 1H), 5.45 (s, 2H), 4.98 (dd, 1H), 2.31-2.30 (m, 2H), 2.00 (dd, 1H), 1.63 (dd, 1H), 1.13 (s, 3H), 0.92 (s, 3H) ppm.

Compound I-33 was synthesized in 66% yield via dehydration of Compound I-20 using sulfuric acid (5 eq, concentrated) in THF.

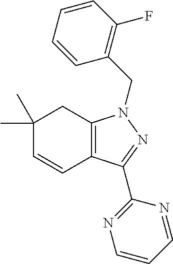 [SMILES: CC1(C)C=Cc2c(-c3ncccn3)nn(Cc3ccccc3F)c2C1]

¹H NMR (400 MHz, CDCl₃) δ 8.80 (d, 2H), 7.28-7.22 (m, 1H), 7.15 (t, 1H), 7.11 (d, 1H), 7.08-7.01 (m, 3H), 5.51 (d, 1H), 5.47 (s, 2H), 2.55 (s, 2H), 1.07 (s, 6H) ppm.

Compound I-21 was synthesized as a light yellow solid (12% yield over 3 steps) using cyclohexane-1,3-dione in step 1 and 2-fluorobenzyl bromide in step 3. [NOTE: In the first step, the electrophile was formed in situ from CDI (1.05 eq) and pyrimidine-2-carboxylic acid (1.0 eq) in CHCl₃ at 40° C. for 2 h, then it was directly subjected to cyclohexane-1,3-dione (1.0 eq) and DMAP (1.0 eq) for 14 h at 80° C. to form desired adduct.]

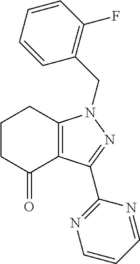 [SMILES: O=C1CCCc2c1c(-c1ncccn1)nn2Cc1ccccc1F]

¹H NMR (400 MHz, CDCl₃) δ 8.89 (d, 2H), 7.32-7.27 (m, 1H), 7.30 (t, 1H), 7.20-7.15 (m, 1H), 7.10-7.06 (m, 2H), 5.46 (s, 2H), 2.84 (t, 2H), 2.54 (t, 2H), 2.22-2.16 (m, 2H) ppm.

Compound I-29 was synthesized as a yellow foam (22% yield over 3 steps) using 4,4-dimethylcyclohex-2-enone in step 1 and 2-fluorobenzyl bromide in step 3.

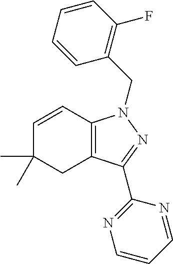 [SMILES: CC1(C)C=Cc2c(c(-c3ncccn3)nn2Cc2ccccc2F)C1]

¹H NMR (400 MHz, CDCl₃) δ 8.80 (dd, 2H), 7.24-7.21 (m, 2H), 7.15 (dt, 1H), 7.07-7.02 (m, 2H), 6.22 (d, 1H), 5.72 (d, 1H), 5.50 (s, 2H), 3.07 (s, 2H), 1.11 (s, 6H) ppm.

Compound I-30 was synthesized as a white solid (1% yield over 3 steps) using 4,4-difluorocyclohexanone in step 1 and 2-fluorobenzyl bromide in step 3.

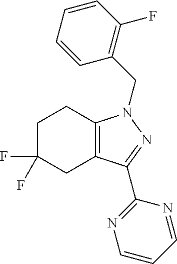 [SMILES: Fc1ccccc1Cn1nc(-c2ncccn2)c2c1CCC(F)(F)C2]

¹H NMR (400 MHz, CDCl₃) δ 8.79 (d, 2H), 7.28-7.24 (m, 1H), 7.17 (t, 1H), 7.09-7.04 (m, 3H), 5.45 (s, 2H), 3.49 (t, 2H), 2.77 (t, 2H), 2.25 (m, 2H) ppm.

Compound I-15 was synthesized as an off-white solid (6% yield over 3 steps) following General Procedure A using dihydro-2H-thiopyran-4(3H)-one in step 1 and 2-fluorobenzyl bromide in step 3.

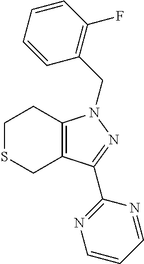 [SMILES: Fc1ccccc1Cn1nc(-c2ncccn2)c2c1CCSC2]

¹H NMR (400 MHz, CDCl₃) δ 8.89 (d, 2H), 7.28-7.19 (m, 3H), 7.10-7.04 (m, 2H), 5.42 (s, 2H), 4.12 (s, 2H), 2.92-2.87 (m, 4H) ppm.

Compound I-19 was synthesized as a white solid (18%) via bis-oxidation of Compound T mediated by mCPBA.

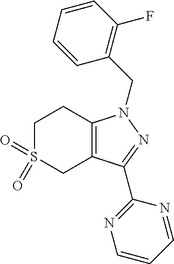 [SMILES: O=S1(=O)CCc2c(c(-c3ncccn3)nn2Cc2ccccc2F)C1]

¹H NMR (400 MHz, CDCl₃) δ 8.89 (d, 2H), 7.34-7.26 (m, 3H), 7.14-7.08 (m, 2H), 5.41 (s, 2H), 4.67 (s, 2H), 3.30-3.28 (m, 4H) ppm.

Compound I-41 was synthesized as a grayish solid (25% yield over 3 steps) using cyclohexanone is step 1 and 2-methoxypyridin-3-yl-methyl bromide as the electrophile in step 3.

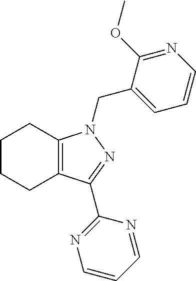 [SMILES: COc1ncccc1Cn1nc(-c2ncccn2)c2c1CCCC2]

¹H NMR (400 MHz, CDCl₃) δ 8.74 (m, 2H), 8.03-8.00 (m, 1H), 7.10 (t, 1H), 6.96-6.92 (m, 1H), 6.75-6.71 (m, 1H), 5.29 (s, 2H), 3.97 (s, 3H), 2.97-2.90 (m, 2H), 2.50-2.43 (m, 2H), 1.83-1.70 (m, 4H). MS: 322.2 (M+1).

Compound I-42 was synthesized as a grayish solid (24% yield over 3 steps) using cyclohexanone in step 1 and 2-chloropyridin-3-yl-methyl in step 3.

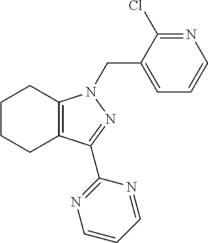 [SMILES: Clc1ncccc1Cn1nc(-c2ncccn2)c2c1CCCC2]

¹H NMR (400 MHz, CDCl₃) δ 8.93-8.87 (m, 2H), 8.42-8.37 (m, 1H), 7.29-7.20 (m, 2H), 7.15-7.10 (m, 1H), 5.56 (s, 2H), 3.13-3.03 (m, 2H), 2.66-2.54 (m, 2H), 2.00-1.83 (m, 4H). MS: 326.8 (M+1).

Compound I-43 was synthesized as a grayish solid (18% yield over 3 steps) using cyclohexanone in step 1 and 2-chlorobenzyl bromide in step 3.

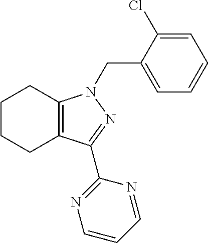 [SMILES: Clc1ccccc1Cn1nc(-c2ncccn2)c2c1CCCC2]

¹H NMR (400 MHz, CDCl₃) δ 8.78-8.74 (m, 2H), 7.35-7.31 (m, 1H), 7.19-7.13 (m, 1H), 7.13-7.07 (m, 2H), 6.70-6.66 (m, 1H), 5.46 (s, 2H), 2.98-2.93 (m, 2H), 2.48-2.41 (m, 2H), 1.84-1.68 (m, 4H). MS: 325.1 (M+1).

Compound I-44 was synthesized as a light yellow solid (19% yield over 3 steps) using cyclohexanone in step 1 and 2-chlorobenzyl bromide in step 3.

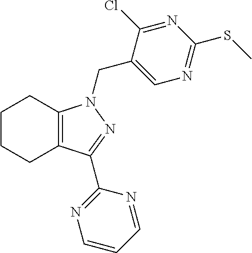 [SMILES: CSc1ncc(Cn2nc(-c3ncccn3)c3c2CCCC3)c(Cl)n1]

¹H NMR (400 MHz, CDCl₃) δ 8.76-8.72 (d, 2H), 7.86 (s, 1H), 7.13-7.08 (t, 1H), 5.34 (s, 2H), 2.96-2.86 (m, 2H), 2.53-2.48 (m, 2H), 2.47 (s, 3H), 1.84-1.69 (m, 4H). MS: 373.1 (M+1).

### Compound I-45

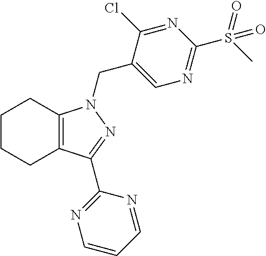 [SMILES: CS(=O)(=O)c1ncc(Cn2nc(-c3ncccn3)c3c2CCCC3)c(Cl)n1]

To a cold solution of compound I-44 (0.304 g, 0.815 mmol) in methanol (5.4 ml) at 0° C., was added a solution of OXONE® (1.50 g, 2.45 mmol) dissolved in water (5.4 ml). The mixture was allowed to warm to room temperature and stirred for additional 4 hours. The mixture was concentrated under vacuum and the resulting residue was dissolved in ethyl acetate (100 ml). The organic layer was washed with saturated solution of sodium bicarbonate (50 ml), brine (50 ml), dried (MgSO₄), filtered, and evaporated to give oil. The crude oil was then purified using SiO₂ chromatography and an appropriate gradient (acetonitrile/methanol) to give compound I-45 as a white solid (32% yield).

¹H NMR (400 MHz, CDCl₃) δ 8.81 (d, 2H), 8.16 (s, 1H), 7.19 (t, 1H), 5.51 (s, 2H), 3.02-2.96 (m, 2H), 2.60-2.54 (m, 2H), 2.17 (s, 3H), 1.94-1.77 (m, 4H).

MS: 405.2 (M+1).

### Compound I-46

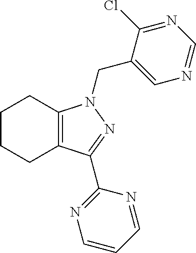 [SMILES: Clc1ncncc1Cn1nc(-c2ncccn2)c2c1CCCC2]

To a mixture of compound I-45 (104 mg, 0.257 mmol) in dichloromethane (1.0 ml) and methanol (1.0 ml), was added sodium borohydride (48.6 mg, 1.284 mmol). The mixture was stirred at 25° C. for 2 h and concentrated under vacuum. It was diluted in ethyl acetate (100 ml) and washed with water (100 ml). The organic layer was dried (MgSO4), filtered, and evaporated to give foam. The crude foam was purified using SiO2 chromatography and an appropriate gradient (acetonitrile/methanol) to give compound I-46 as a white solid (32% yield).

¹H NMR (400 MHz, CDCl₃) δ 8.91 (s, 1H), 8.80 (d, 2H), 8.02 (s, 1H), 7.17 (t, 1H), 5.46 (s, 2H), 3.02-2.95 (m, 2H), 2.59-2.51 (m, 2H), 1.92-1.76 (m, 4H).

MS: 327.1 (M+1).

### Example 2

### General Procedure B

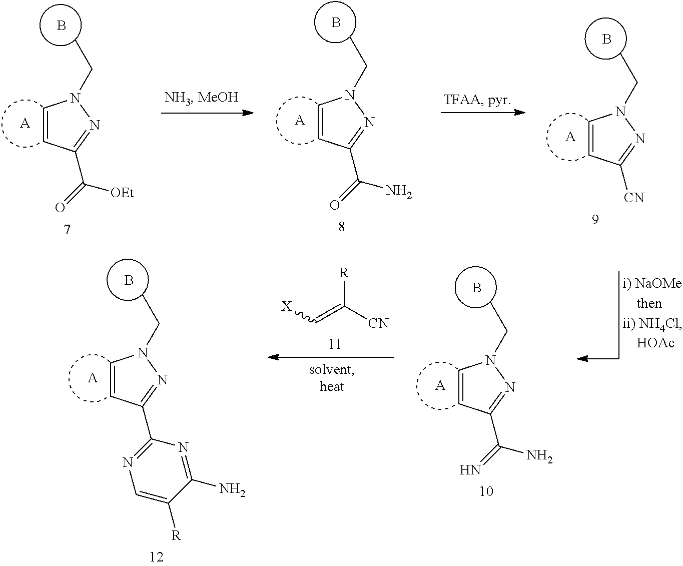

NOTE: Pyrazole 7 is generated in an analogous fashion to pyrazole 6 from General Procedure A, except using diethyl oxalate as reagent 2.

### Step 1: Primary Amide Formation

In a high-pressure glass tube, ethyl ester 7 was directly charged with a 7N solution of ammonia in methanol (large excess, >30 eq) and a catalytic amount of NaCN (0.10 eq). The reaction was then heated and stirred at 90° C. until reaction was judged complete by LC/MS analysis. Once complete, reaction was concentrated and the resulting material is diluted with DCM and filtered. The filtrate was concentrated and the crude oil was then purified using SiO₂ chromatography and an appropriate gradient (ethyl acetate/hexanes or DCM/methanol) to give amide 8, typically as a white foam.

### Step 2: Nitrite Formation

Amide 8 was dissolved in pyridine (0.25M) and cooled to 0° C. Trifluoroacetic anhydride was then added (fuming upon addition) and the reaction was closely monitored by LC/MS analysis. Once complete, reaction was diluted with DCM and washed with water. The aqueous portion was back extracted with additional DCM (×2). The organic portions were then combined, dried (Na₂SO₄), filtered, and concentrated. The crude oil was then purified using SiO₂ chromatography and an appropriate gradient (ethyl acetate/hexanes or DCM/methanol) to give nitrile 9, typically as a white foam.

### Step 3: Carboximidamide Formation

Nitrile 9 was added to a solution of sodium methoxide (1.25 eq) in methanol. Reaction was treated to 40° C. and stirred for 3 hours. At this time, acetic acid (10 eq) and ammonium chloride (3 eq) are added and the reaction is stirred at reflux for 12-16 h. At this time, reaction is directly concentrated, and the remaining crude material is diluted with EtOAc and basified by the addition of a saturated solution of sodium carbonate. The heterogeneous mixture was transferred to a separatory funnel where the layers were separated. The aqueous portion was then extracted an addition two times with DCM. The organic portions were then combined, dried (Na₂SO₄), filtered, and concentrated. The crude carboximidamide 10 was carried directly on to the cyclization reaction to generate the targeted pyrimidine.

### Step 4: Pyrimidine Formation

Carboximidamide 10 was dissolved in an appropriate solvent (xylene, toluene, or pyridine) and charged with vinyl nitrile 11. Reaction is then capped and heated at reflux until >90% complete by LC/MS analysis. Reaction is then concentrated, taken back up in DCM, and extracted with water. The aqueous portion was then extracted an addition two times with DCM. The organic portions were then combined, dried (Na₂SO₄), filtered, and concentrated. The crude oil was purified by reverse phase, preparative HPLC to give pyrimidine 12, as a (color) solid or liquid, etc.
The following compounds were synthesized following General Procedure B using the appropriate vinyl nitrile 11 and solvent in step 4.

Compound I-3 was synthesized as a white solid (<2% overall yield over 7 steps) using 3-ethoxyacrylonitrile as the vinyl nitrile and ethanol (also added two equiv of sodium methoxide) as solvent in step 4.

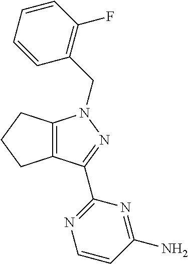 [SMILES: Nc1ccnc(-c2nn(Cc3ccccc3F)c3c2CCC3)n1]

¹H NMR (400 MHz, CDCl₃) δ 8.30 (d, 1H), 7.29-7.23 (m, 1H), 7.13 (ddd, 1H), 7.09-7.03 (m, 2H) 6.28 (d, 1H), 5.39 (s, 2H), 4.96 (bs, 2H), 2.92-2.88 (m, 2H), 2.52-2.48 (m, 4H) ppm.

Compound I-4 was synthesized as a white solid (<2% overall yield over 7 steps) following General Procedure B. The cyclization was carried out using the nitrile intermediate with biguanide (1.0 eq) in the presence of sodium methoxide (1.0 eq), using ethanol as solvent.

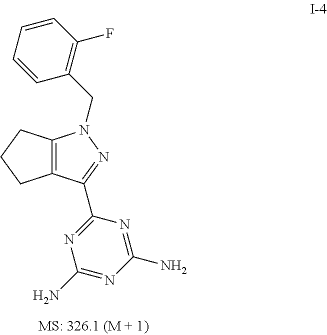 [SMILES: Nc1nc(N)nc(-c2nn(Cc3ccccc3F)c3c2CCC3)n1]

Compound I-28 was synthesized as a white solid (1% overall yield over 7 steps) using 3-ethoxyacrylonitrile as the vinyl nitrile and toluene as solvent in step 4.

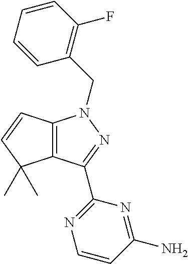 [SMILES: CC1(C)C=Cc2c1c(-c1nccc(N)n1)nn2Cc1ccccc1F]

¹H NMR (400 MHz, CD₃OH) δ 7.86 (d, 1H), 7.32-7.20 (m, 2H), 7.10-7.02 (m, 2H), 6.50 (d, 1H), 6.44 (d, 1H), 6.29 (d, 1H), 5.41 (s, 2H), 1.23 (s, 6H) ppm.

Compound I-31 was synthesized as a white solid (1.4% overall yield over 7 steps) using 3-ethoxyacrylonitrile as the vinyl nitrile and toluene as solvent in step 4.

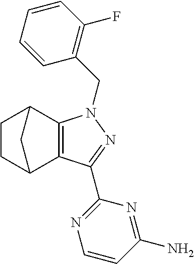 [SMILES: Nc1ccnc(-c2nn(Cc3ccccc3F)c3c2C2CCC3C2)n1]

¹H NMR (400 MHz, CD₃OD) δ 7.96 (d, 1H), 7.43-7.34 (m, 2H), 7.22-7.14 (m, 2H), 6.60 (d, 1H), 5.51 (d, 1H), 5.45 (d, 1H), 3.76-3.75 (m, 1H), 3.36-3.34 (m, 1H), 2.03-1.91 (m, 2H), 1.89-1.81 (m, 1H), 1.70-1.68 (m, 1H), 1.11-1.05 (m, 1H), 0.96-0.90 (m, 1H) ppm.

Compound I-22 was synthesized as a white solid (3% overall yield over 7 steps) following General Procedure B using 3-ethoxyacrylonitrile as the vinyl nitrile and toluene as solvent in step 4.

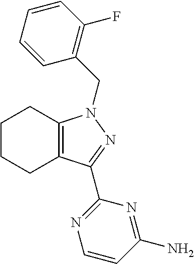 [SMILES: Nc1ccnc(-c2nn(Cc3ccccc3F)c3c2CCCC3)n1]

¹H NMR (400 MHz, CD₃OH) δ 7.97 (t, 1H), 7.36-7.32 (m, 1H), 7.16-7.11 (m, 3H), 6.59 (d, 1H), 5.44 (s, 2H), 4.90 (s, 2H), 2.92 (t, 2H), 2.64 (t, 2H), 1.87-1.82 (m, 2H), 1.80-1.76 (m, 2H) ppm.

Compound I-27 was synthesized as a white solid (2% overall yield over 7 steps) following General Procedure B using 3-(dimethylamino)-2-(pyridin-4-yl)acrylonitrile as the vinyl nitrile and xylene as solvent in step 4.

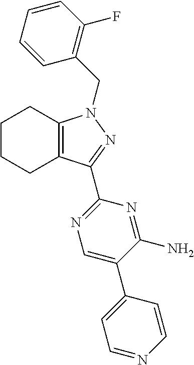 [SMILES: Nc1nc(-c2nn(Cc3ccccc3F)c3c2CCCC3)ncc1-c1ccncc1]

¹H NMR (400 MHz, CDCl₃) δ 8.93-8.91 (m, 2H), 8.62 (s, 1H), 7.85-7.83 (m, 2H), 7.54-7.49 (m, 1H), 7.44-7.40 (m, 1H), 7.33-7.28 (m, 2H), 5.90-5.87 (b, 2H), 5.57 (s, 2H), 3.03 (t, 2H), 2.85 (t, 2H), 2.01-1.95 (m, 2H), 1.92-1.87 (m, 2H) ppm.

### Example 3

### General Procedure C

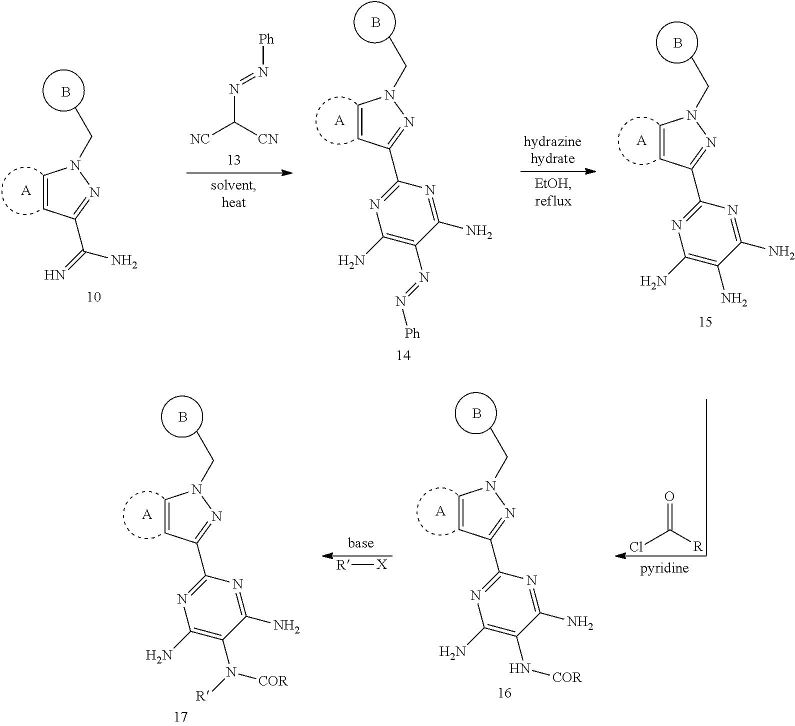

### Step 1: Pyrimidine Formation

Carboximidamide 10 was dissolved in toluene (or DMF) and charged with NaOMe (1-2 eq). 2-(Phenyldiazenyl)malononitrile 13 (1.1 eq) was added, and the reaction vessel was then capped and heated at 100° C. until >90% complete by LC/MS analysis. Reaction was then diluted with DCM and extracted with NH₄Cl (conc., aq). The aqueous portion was then extracted an addition two times with DCM. The organic portions were then combined, dried (Na₂SO₄), filtered, and concentrated. The crude oil was purified by either reverse phase, preparative HPLC or by normal phase chromatography and a methanol/DCM gradient to give desired pyrimidine 14.

### Step 2: Hydrazinolysis

To a solution of pyrimidine 14 in EtOH was added hydrazine hydrate (>50 eq). Reaction mixture was then heated to reflux and stirred 14-48 h, or until reaction is judged complete by LC/MS analysis. The crude reaction was then concentrated and purified by either reverse phase, preparative HPLC or by normal phase chromatography and a methanol/DCM gradient to give desired pyrimidine 15.

### Step 3: Acyclation

Tri-amino pyrimidine 15 was dissolved in pyridine and cooled to 0° C., at which time the acylating reagent (acyl chloride, chloroformate, etc., 1.0 eq) was added. The reaction was stirred at 0° C. until judged complete by LC/MS analysis (typically <2 h min). The crude reaction was then concentrated and purified by either reverse phase, preparative HPLC or by normal phase chromatography and a methanol/DCM gradient to give desired pyrimidine 16.

### Step 4: Alkylation

Pyrimidine 16 was dissolved in solvent (most typically DMF) and cooled to 0° C. Sodium hydride (1.2 eq) was added followed by the electrophile (intramolecular variants do not require exogenous electrophiles), and the resulting reaction was closely monitored by LC/MS analysis. Once complete, the reaction was quenched with water and extracted with DCM (3×). The organic portions were then combined, dried (Na₂SO₄), filtered, and concentrated. The crude oil was purified by either reverse phase, preparative HPLC or by normal phase chromatography and a methanol/DCM gradient to give desired pyrimidine 17.
The following compounds were synthesized following General Procedure C using the appropriate acylating group in step 3 and alkylating agent in step 4.

Compound I-34 was synthesized as an orange solid (51% yield from the corresponding carboximidamide) following General Procedure C.

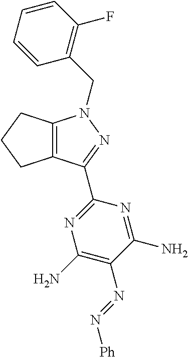 [SMILES: Nc1nc(-c2nn(Cc3ccccc3F)c3c2CCC3)nc(N)c1/N=N/c1ccccc1]

¹H NMR (400 MHz, DMSO d₆) δ 8.34 (bs, 2H), 7.95-7.93 (m, 2H), 7.51-7.10 (m, 9H), 5.29 (s, 2H), 2.83 (t, 2H), 2.62 (t, 2H), 2.48-2.45 (m, 2H) ppm.

Compound I-35 was synthesized as an orange solid (13% yield in two steps from the corresponding carboximidamide) following General Procedure C.

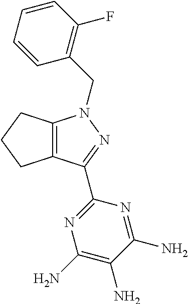 [SMILES: Nc1nc(-c2nn(Cc3ccccc3F)c3c2CCC3)nc(N)c1N]

¹H NMR (400 MHz, CD₃OD) δ 7.25-7.20 (m, 1H), 7.12 (ddd, 1H), 7.05-6.97 (m, 2H), 5.22 (s, 2H), 2.71 (t, 2H), 2.53-2.49 (m, 2H), 2.45-2.40 (m, 2H) ppm.

Compound I-36 was synthesized as an off-white solid (10% yield in three steps from the corresponding carboximidamide) following General Procedure C using methyl chloroformate as an acylating agent in step 3.

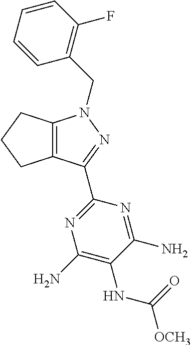 [SMILES: COC(=O)Nc1c(N)nc(-c2nn(Cc3ccccc3F)c3c2CCC3)nc1N]

¹H NMR (400 MHz, CD₃OD) δ 7.36-7.30 (m, 1H), 7.19 (ddd, 1H), 7.14-7.09 (m, 2H), 5.31 (s, 2H), 3.73 (s, 3H), 2.84 (t, 2H), 2.59-2.48 (m, 4H) ppm.

Compound I-37 was synthesized as an off-white solid (3% yield in four steps from the corresponding carboximidamide) following General Procedure C using methyl chloroformate as an acylating agent in step 3 and methyl iodide as an alkylating agent in step 4.

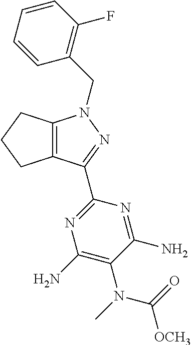 [SMILES: COC(=O)N(C)c1c(N)nc(-c2nn(Cc3ccccc3F)c3c2CCC3)nc1N]

¹H NMR (400 MHz, CD₃OD) δ 7.41-7.36 (m, 1H), 7.28 (ddd, 1H), 7.18 (ddd, 1H), 7.17-7.13 (m, 1H), 5.40 (s, 2H), 3.81/3.68 (s/s, 3H rotomeric methyl group), 3.12/3.10 (s/s, 3H rotomeric methyl group), 2.89-2.86 (m, 2H), 2.69-2.66 (m, 2H), 2.60-2.56 (m, 2H) ppm.

Compound I-12 was synthesized as an orange/red solid (18% yield in two steps from the corresponding carboximidamide) following General Procedure C.

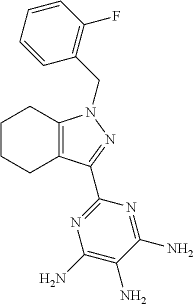 [SMILES: Nc1nc(-c2nn(Cc3ccccc3F)c3c2CCCC3)nc(N)c1N]

¹H NMR (400 MHz, CDCl₃) δ 7.15-7.11 (m, 1H), 6.97-6.89 (m, 2H), 6.82-6.79 (m, 1H), 5.25 (s, 2H), 5.20 (bs, 4H), 2.94-2.92 (m, 2H), 2.79 (bs, 2H), 2.39 (bs, 2H), 1.71-1.66 (m, 4H) ppm.

Compound I-13 was synthesized as an orange/red solid (14% yield in three steps from the corresponding carboximidamide) following General Procedure C using methyl chloroformate as an acylating agent in step 3.

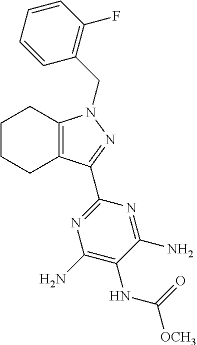 [SMILES: COC(=O)Nc1c(N)nc(-c2nn(Cc3ccccc3F)c3c2CCCC3)nc1N]

¹H NMR (400 MHz, CD₃OD) δ 7.21-7.15 (m, 1H), 7.01-6.94 (m, 2H), 6.89-6.82 (m, 1H), 5.23 (s, 2H), 3.63 (bs, 3H), 2.72 (t, 2H), 2.44 (t, 2H), 1.70-1.59 (m, 4H) ppm.

Compound I-14 was synthesized as an off-white solid (5% yield in four steps from the corresponding carboximidamide) following General Procedure C using methyl chloroformate as an acylating agent in step 3 and methyl iodide as an alkylating agent in step 4.

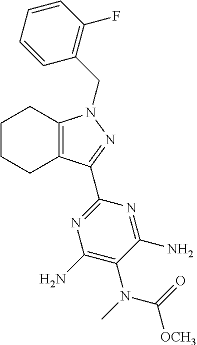 [SMILES: COC(=O)N(C)c1c(N)nc(-c2nn(Cc3ccccc3F)c3c2CCCC3)nc1N]

¹H NMR (400 MHz, CDCl₃) δ 12.61 (bs, 2H), 7.32-6.92 (m, 6H), 5.27 (bs, 2H), 3.64 (bs, 3H), 3.11 (s, 3H), 2.74 (bs, 2H), 2.53 (bs, 2H), 1.77 (bs, 2H), 1.68 (bs, 2H) ppm.

Compound I-25 was synthesized as an off-white solid (18% yield in three steps from the corresponding carboximidamide) using chloroethyl chloroformate as an acylating agent in step 3.

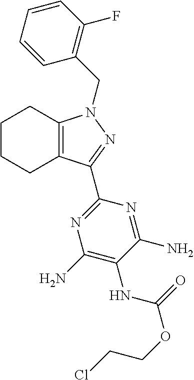 [SMILES: Nc1nc(-c2nn(Cc3ccccc3F)c3c2CCCC3)nc(N)c1NC(=O)OCCCl]

¹H NMR (400 MHz, CD₃OD) δ 7.24-7.18 (m, 1H), 7.03-6.92 (m, 2H), 6.89-6.85 (m, 1H), 5.29 (s, 2H), 4.28-4.22 (m, 2H), 3.70-3.63 (m, 2H), 2.75 (t, 2H), 2.49 (t, 2H), 1.72-1.61 (m, 4H) ppm.

Compound I-26 was synthesized as an off-white solid (15% yield in four steps from the corresponding carboximidamide) using chloroethyl chloroformate as an acylating agent in step 3 and the resulting chloroethyl chain as an alkylating agent in step 4.

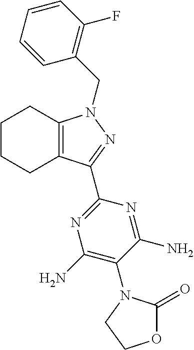 [SMILES: Nc1nc(-c2nn(Cc3ccccc3F)c3c2CCCC3)nc(N)c1N1CCOC1=O]

¹H NMR (400 MHz, CD₃OD) δ 7.27-7.21 (m, 1H), 7.05-7.01 (m, 2H), 6.96 (dd, 1H), 5.32 (s, 2H), 4.48 (t, 2H), 3.67 (t, 2H), 2.78 (t, 2H), 2.52 (t, 2H), 1.75-1.64 (m, 4H) ppm.

Compound I-23 was synthesized as an off-white solid (2% yield in four steps from the corresponding carboximidamide) following General Procedure C using methyl chloroformate as an acylating agent in step 3 and 2-fluorobenzyl bromide as an alkylating agent in step 4.

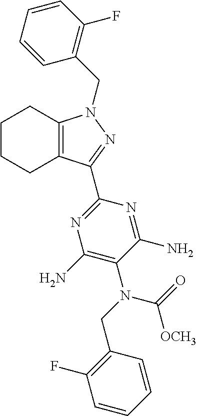 [SMILES: COC(=O)N(Cc1ccccc1F)c1c(N)nc(-c2nn(Cc3ccccc3F)c3c2CCCC3)nc1N]

¹H NMR (400 MHz, CDCl₃) δ 7.45-7.42 (m, 2H), 7.34-7.28 (m, 6H), 7.14-7.12 (m, 2H), 7.11-7.01 (m, 2H), 5.53 (s, 2H), 4.78 (s, 2H), 3.78 (s, 3H), 2.72 (t, 2H), 2.56 (t, 2H), 1.80-1.76 (m, 2H), 1.71-1.67 (m, 2H) ppm.

Compound I-24 was synthesized as an off-white solid (4% yield in four steps from the corresponding carboximidamide) following General Procedure C using methyl chloroformate as an acylating agent in step 3 and 2-fluorobenzyl bromide as an alkylation agent in step 4—over-alkylation product.

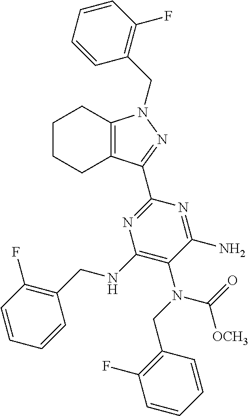 [SMILES: COC(=O)N(Cc1ccccc1F)c1c(N)nc(-c2nn(Cc3ccccc3F)c3c2CCCC3)nc1NCc1ccccc1F]

¹H NMR (400 MHz, CDCl₃) δ 7.45-7.42 (m, 1H), 7.38-7.34 (m, 1H), 7.30-7.21 (m, 5H), 7.14-7.01 (m, 6H), 6.91-6.86 (m, 1H), 5.46 (m, 1H), 5.30 (s, 2H), 4.92 (d, 1H), 4.65-4.57 (m, 3H), 3.75 (s, 3H), 2.62-2.54 (m, 4H), 1.78-1.73 (m, 2H), 1.64-1.60 (m, 2H) ppm.

### Example 4

### Preparation of Compounds wherein Ring A is a Non-Aromatic Heterocycle

### Step 1: Enamine Formation

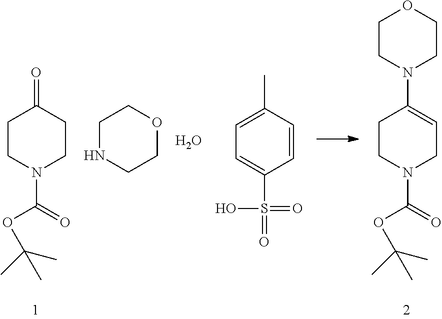

1-Boc-3-piperidone 1 (1 g, 5.02 mmol) was dissolved in benzene (50 ml) and stirred at room temperature. Morpholine (0.437 ml, 5.02 mmol) and p-toluenesulfonic acid monohydrate (0.095 g, 0.502 mmol) were added. A Dean-Stark trap and reflux condenser were attached to the reaction flask and the temperature heated to 100° C. The Dean-Stark trap was wrapped in aluminum foil/cotton to ensure evaporation of benzene. The reaction was refluxed overnight. The next day the reaction was directly concentrated to afford the enamine, tert-butyl 4-morpholino-5,6-dihydropyridine-1(2H)-carboxylate 2, as a yellow oil. This crude material was taken to dione formation without any further purification. NMR shows a mixture of rotamers, presumably because of rotation about the amide bond. ¹H NMR (CDCl₃/400 MHz): δ (ppm) 4.56 (br. s, 1H), 3.94 (br. s, 2H), 3.78-3.81 (m, 1H), 3.74 (t, 4H), 3.54 (t, 2H), 3.1 (t, 1H), 2.80 (t, 4H), 2.22 (br. s, 2H), 1.46 (s, 9H).

### Step 2: Acid Chloride Formation

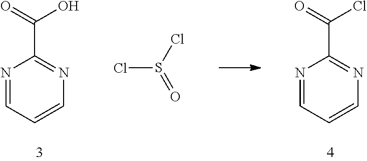

Pyrimidine-2-carboxylic acid 3 (0.310 g, 2.5 mmol) was charged to a vial. Thionyl chloride (3 ml, 41.1 mmol) was added while the mixture was stirred. After addition, the reaction was heated to 110° C. for 1.5 hr to afford a green-colored solution. At this time the heat was removed and the reaction mixture was concentrated in vacuo. The reaction was diluted with 10 ml of toluene and azeotroped twice before drying under high vacuum. After 1 hr, the resulting green/gray residue, pyrimidine-2-carbonyl chloride 4, was carried forward to the dione formation step without any further purification.

### Step 3: Dione Formation

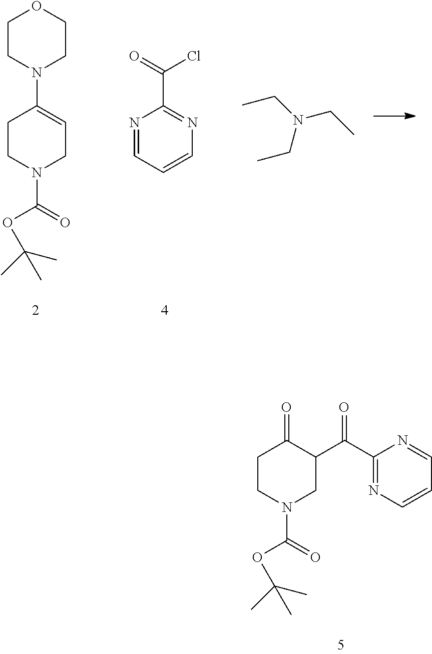

To a stirring solution of tert-butyl 4-morpholino-5,6-dihydropyridine-1(2H)-carboxylate 2 (0.671 g, 2.5 mmol) in 5 ml of 1,2-dichloroethane at 0° C. was added triethylamine (1.742 ml, 12.50 mmol). To this solution was added pyrimidine-2-carbonyl chloride 4 (0.356 g, 2.5 mmol) dissolved in 3 ml 1,2-dichloroethane via syringe. This was added dropwise over the course of 30 minutes. The reaction was allowed to come to room temperature and stirred overnight. The next day 25 ml 1N HCl was added and stirred for 8 hr (pH was approximately =1). The aqueous layer was extracted 3× with EtOAc. The combined organic layers were washed with brine, dried over sodium sulfate and concentrated in vacuo to afford a viscous, dark green oil which by LCMS indicated tert-butyl 4-oxo-3-(pyrimidine-2-carbonyl)piperidine-1-carboxylate 5. This crude mixture was taken on to the pyrazole cyclization step without any further purification.

MS: 304.1 (M−1)

### Step 4: Pyrazole Formation

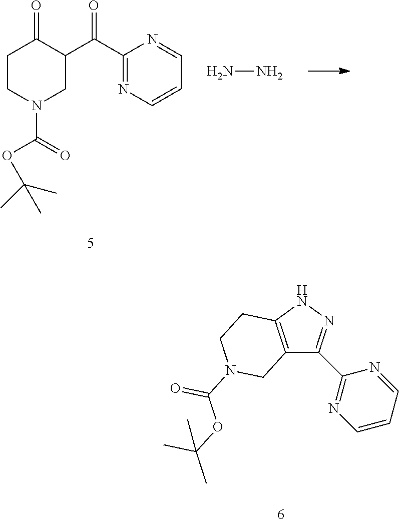

To a stirring solution of tert-butyl 4-oxo3-(pyrimidine-2-carbonyl)piperidine-1-carboxylate 5 (0.545 g, 1.785 mmol) crude in ethanol (5 ml) at 0° C. was added hydrazine (0.067 ml, 2.142 mmol). The reaction was allowed to warm to room temperature overnight. The next day the reaction was concentrated and purified directly by silica gel chromatography using ethyl acetate and hexanes as eluents to afford a slightly yellow solid, tert-butyl 3-(pyrimidin-2-yl)-6,7-dihydro-1H-pyrazolo[4,3]pyridine-5(4H)-carboxylate 6 which is taken forward to the alkylation without any further purification. NMR shows a mixture of rotamers, presumably because of rotation about the amide bond.

¹H NMR (CDCl₃/400 MHz): δ (ppm) 11.05 (br. s, 1H), 8.75 (br. s, 2H), 7.16 (br. s, 1H), 4.85 (br. s, 2H), 3.75 (br. s, 2H), 3.60 (t, 1H), 3.51 (t, 1H), 2.82 (t, 2H), 2.57 (t, 1H), 2.45 (t, 1H), 1.50 (s, 9H). MS: 300.1 (M−1). MS: 302.2 (M+1), 202.1 (M+1).

### Step 5: Alkylation: Compound I-38

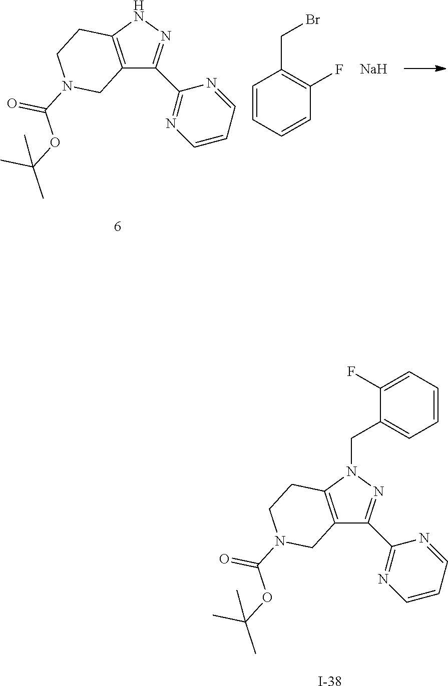

To a stirring solution of tert-butyl 3-(pyrimidin-2-yl)-6,7-dihydro-1H-pyrazolo[4,3]pyridine-5(4H)-carboxylate 6 (0.240 g, 0.796 mmol) in THF (10 ml) was added sodium hydride (0.035 g, 0.876 mmol) generating effervescence. The reaction was lifted from the ice bath and stirred at room temperature for 30 minutes. 1-(bromomethyl)-2-fluorobenzene (0.106 ml, 0.876 mmol) was added and stirred over night. The next day the reaction was complete and was quenched with brine and stirred for 10 minutes. The aqueous layer was extracted 3× with EtOAc. The combined organic layers were washed with brine, dried over sodium sulfate and concentrated in vacuo. The resulting crude mixture was purified by silica gel chromatography using DCM, MeOH and ACN as eluents. The desired product is a white solid which shows tert-butyl 1-(2-fluorobenzyl)-3-(pyrimidin-2-yl)-6,7-dihydro-1H-pyrazolo[4,3]pyridine-5(4H)-carboxylate, compound I-38 (0.185 g, 0.452 mmol, 56.7% yield) by NMR.

¹H NMR (CDCl₃/400 MHz): δ (ppm) 8.7 (d, 2H), 7.22-7.26 (m, 1H), 7.13 (t, 1H), 7.02-7.10 (m, 3H), 5.44 (s, 2H), 4.81 (br. S, 2H), 3.69 (br. S, 2H), 2.61 (br. S, 2H), 1.47 (s, 9H). MS: 410.2 (M+1), 310.2 (M+1).

### Step 6: Deprotection: Compound I-39

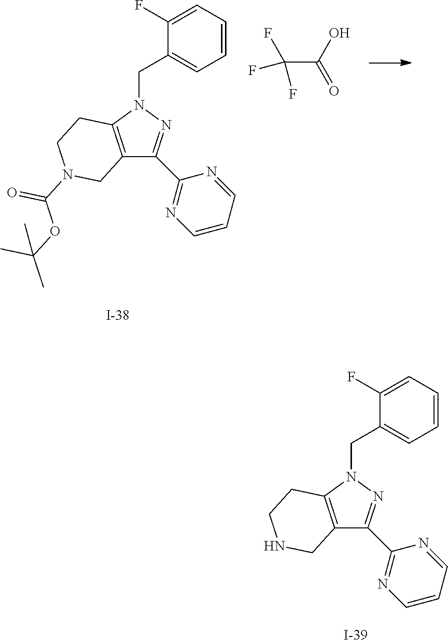

To a stirring solution of tert-butyl 1-(2-fluorobenzyl)-3-(pyrimidin-2-yl)-6,7-dihydro-1H-pyrazolo[4,3]pyridine-5(4H)-carboxylate, I-38 (0.165 g, 0.403 mmol) in DCM (5 ml) was added trifluoroacetic acid (0.310 ml, 4.03 mmol). The reaction was stirred overnight at room temperature. The next day the reaction was complete and was quenched with aqueous sodium bicarbonate and stirred for 10 minutes. The reaction was diluted with EtOAc. The aqueous layer was extracted 3× with EtOAc. The combined organic layers were washed with brine, dried over sodium sulfate and concentrated in vacuo. The mixture was then taken up in methanol and purified by C18 reverse phase chromatography. The desired fractions were concentrated and dried. NMR shows 1-(2-fluorobenzyl)-3-(pyrimidin-2-yl)-4,5,6,7-tetrahydro-1H-pyrazolo[4,3-c]pyridine, I-39 (0.048 g, 0.155 mmol, 38.5% yield) as a white solid.

¹H NMR (CDCl₃/400 MHz): δ (ppm) 8.77 (d, 2H), 7.23-7.26 (m, 1H), 7.13 (t, 1H), 7.05-7.08 (m, 3H), 5.45 (s, 2H), 4.24 (s, 2H), 3.11 (t, 2H), 2.55 (t, 2H). MS: 310.2 (M+1).

### Step 7: Amide Bond Formation: Compound I-40

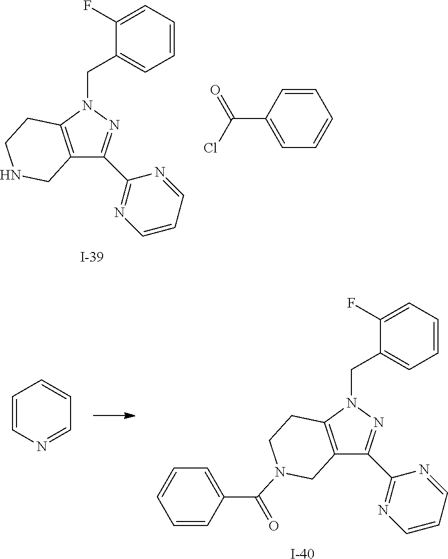

To a stirring solution of 1-(2-fluorobenzyl)-3-(pyrimidin-2-yl)-4,5,6,7-tetrahydro-1H-pyrazolo[4,3-c]pyridine (I-39, 0.048 g, 0.155 mmol) in DCM (2 ml) was added pyridine (0.019 ml, 0.233 mmol) and benzoyl chloride (0.022 ml, 0.186 mmol). The reaction was stirred overnight at rt. The next day the reaction was complete and was concentrated to yield a white solid which was then directly purified by silica gel chromatography using DCM/MeOH/ACN as eluents to yield (1-(2-fluorobenzyl)-3-(pyrimidin-2-yl)-6,7-dihydro-1H-pyrazolo[4,3-c]pyridin-5(4H)-yl)(phenyl)methanone, I-40 (0.039 g, 0.094 mmol, 60.8% yield).

¹H NMR: (CDCl₃/400 MHz): δ (ppm) 8.66 (d, 2H), 7.35-7.40 (m, 4H), 7.19-7.23 (m, 2H), 7.06-7.10 (m, 2H), 6.98-7.03 (m, 2H), 5.39 (s, 2H), 4.90 (d, 2H), 3.98-3.99 (m, 1H), 3.58 (s, 1H), 2.65 (d, 2H). MS: 414.2 (M+1)

### Example 5

### General Procedure D

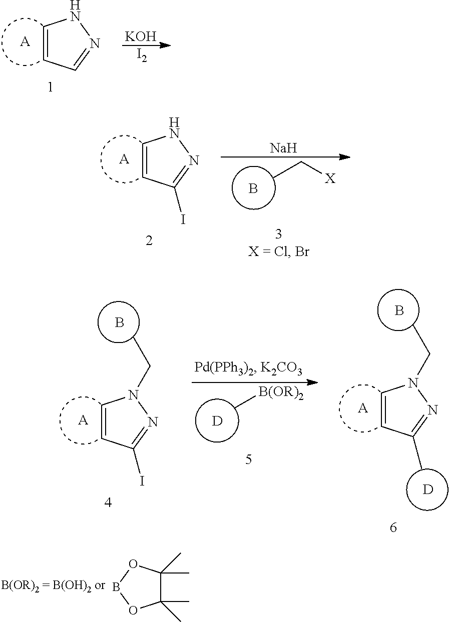

### Step 1: Iodination

To a solution of pyrazole 1 in DMF, was added potassium hydroxide (2.0 eq). The reaction was briefly sonicated for 5 min to help dissolution. Iodine (1.25 eq) was then added and the reaction mixture was stirred until complete (using TLC and LC/MS analysis). Additional portion of iodine (ca. 0.25 eq) could be added to drive reaction to completion. Once completed, the reaction was diluted with water and quenched with saturated sodium thiosulfate. The resulting crude mixture was transferred to a separatory funnel and extracted two times with EtOAc. The organic portions were then combined, washed three times with water and one time with brine, dried (Na₂SO₄), filtered, and concentrated. The crude material was purified using SiO₂ chromatography and an appropriate gradient (ethyl acetate/hexanes or DCM/methanol) to give compound 2, as a solid or liquid.

### Step 2: Alkylation

To a solution of pyrazole 2 in THF was added NaH (1.2 eq, 60% in dispersion oil) portion-wise (bubbling). After stirring at rt for 30 min, electrophile 3 (1.2 eq) was added and the reaction was stirred at rt until completion by LC/MS analysis. Once completed, the reaction was quenched with NH₄Cl, diluted with water and transferred to a separatory funnel. The crude mixture was extracted two times with EtOAc. The organic portions were then combined, dried (Na₂SO₄), filtered, and concentrated. The crude oil was then purified using SiO₂ chromatography and an appropriate gradient (ethyl acetate/hexanes or DCM/methanol) to give compound 4, as a solid or liquid.

### Step 3: Cross Coupling

To a solid mixture of pyrazole 4, boronic acid or ester 5 (1.5 eq), potassium carbonate (2.0 eq) and tetrakis(triphenphenylphosphine)palladium(0) (0.10 eq) under a nitrogen atmosphere in a sealed tube was added DME/MeOH/DMF (2:3:1 ratio). The resulting suspension was heated at 120° C. until completion by LC/MS analysis. Once complete, the reaction was diluted with EtOAc and filtered. The crude mixture was washed sequentially with 1N NaOH solution, water and brine, dried (Na₂SO₄), filtered, and concentrated. The crude material was then purified using SiO₂ chromatography and an appropriate gradient (ethyl acetate/hexanes or DCM/methanol) to give compound 6, as a solid or liquid.
The following compounds were prepared according to General procedure D:

### Compound I-47: 1-(2-fluorobenzyl)-3-(pyrimidin-5-yl)-4,5,6,7-tetrahydro-1H-indazole

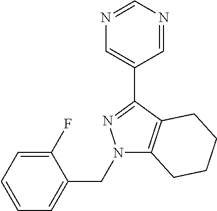 [SMILES: Fc1ccccc1Cn1nc(-c2cncnc2)c2c1CCCC2]

¹H NMR: δ 9.12 (s, 1H), 9.10 (s, 2H), 7.29 (m, 1H), 7.05 (m, 3H), 5.33 (s, 2H), 2.72 (app. t, 2H), 2.57 (app. t, 2H), 1.82 (m, 4H). MS: 309.3 (M+1)

### Compound I-48: 1-(2-fluorobenzyl)-3-(pyridin-3-yl)-4,5,6,7-tetrahydro-1H-indazole

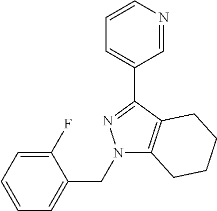 [SMILES: Fc1ccccc1Cn1nc(-c2cccnc2)c2c1CCCC2]

¹H NMR: δ 8.97 (d, 1H), 8.52 (dd, 1H), 8.07 (m, 1H), 7.28 (m, 2H), 7.10 (m, 3H), 5.33 (s, 2H), 2.73 (app. t, 2H), 2.55 (app. t, 2H), 1.82 (m, 4H). MS: 308.2 (M+1)

### Compound I-49: 1-(2-fluorobenzyl)-3-(pyridin-4-yl)-4,5,6,7-tetrahydro-1H-indazole

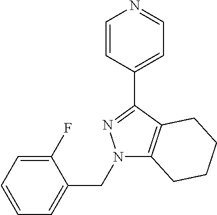 [SMILES: Fc1ccccc1Cn1nc(-c2ccncc2)c2c1CCCC2]

¹H NMR: δ 8.60 (d, 2H), 7.66 (d, 2H), 7.18 (m, 1H), 7.08 (m, 2H), 7.00 (app. t, 1H), 5.34 (s, 2H), 2.76 (app. t, 2H), 2.55 (app. t, 2H), 1.83 (m, 4H). MS: 308.2 (M+1)

### Example 6

### Exemplification of the Synthesis of Compounds Involving Sulfonamide, Amide or Urea Formation

### Compound I-53: 1-(2-fluorobenzyl)-5-(phenylsulfonyl)-3-(pyrimidin-2-yl)-4,5,6,7-tetrahydro-1H-pyrazolo[4,3-c]pyridine

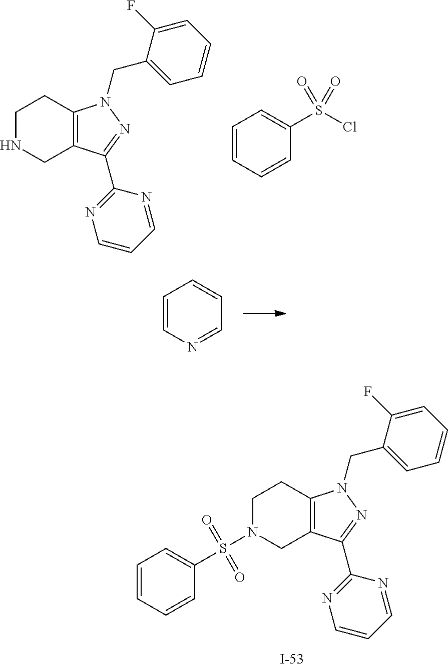

### Sulfonamide Formation:

To a stirring solution of 1-(2-fluorobenzyl)-3-(pyrimidin-2-yl)-4,5,6,7-tetrahydro-1H-pyrazolo[4,3-c]pyridine (0.065 g, 0.210 mmol) in DCE (Volume: 3 ml) in a green-capped vial was added pyridine (0.035 ml, 0.433 mmol) and benzenesulfonyl chloride (0.033 ml, 0.260 mmol). The homogeneous yellow reaction was stirred overnight. Next day lcms indicated formation of desired mass and consumption of starting material. The reaction was concentrated and purified directly by silica gel chromatography. The final product eluted at 100% EtOAc and afforded 1-(2-fluorobenzyl)-5-(phenylsulfonyl)-3-(pyrimidin-2-yl)-4,5,6,7-tetrahydro-1H-pyrazolo[4,3-c]pyridine, Compound I-53 (0.0135 g, 0.030 mmol, 13.88% yield) as a hybrid between an oil and solid. ¹H NMR (CDCl₃/400 MHz): δ (ppm) 8.81 (d, 2H), 7.83-7.86 (m, 2H), 7.52-7.56 (m, 1H), 7.45-7.50 (m, 2H), 7.23-7.29 (m, 1H), 7.18 (t, 1H), 6.99-7.04 (m, 3H), 5.38 (s, 2H), 4.67 (s, 2H), 3.48 (t, 2H), 2.63 (t, 2H). MS: 450.1 (M+1).

Compound I-51: 1-(2-fluorobenzyl)-5-(methylsulfonyl)-3-(pyrimidin-2-yl)-4,5,6,7-tetrahydro-1H-pyrazolo[4,3-c]pyridine

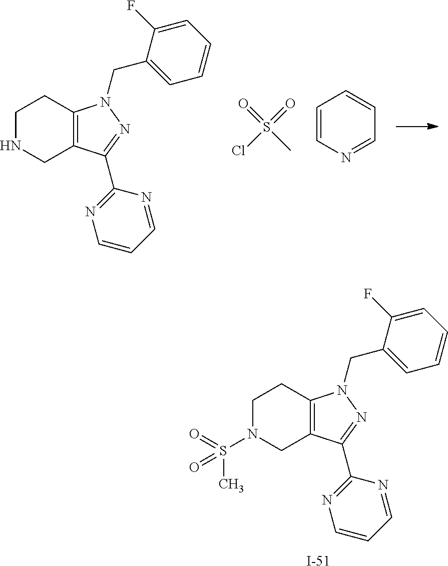

### Sulfonamide Formation:

To a stirring solution of 1-(2-fluorobenzyl)-3-(pyrimidin-2-yl)-4,5,6,7-tetrahydro-1H-pyrazolo[4,3-c]pyridine (0.060 g, 0.194 mmol) in DCE (Volume: 3 ml) was added pyridine (0.042 g, 0.535 mmol) and methanesulfonyl chloride (0.02 ml, 0.257 mmol). The reaction was stirred overnight to afford a yellow, heterogeneous reaction mixture. The reaction was concentrated and purified directly by silica gel chromatography using EtOAc and hexanes as eluents. The desired product eluted at 100% EtOAc. NMR shows 1-(2-fluorobenzyl)-5-(methylsulfonyl)-3-(pyrimidin-2-yl)-4,5,6,7-tetrahydro-1H-pyrazolo[4,3-c]pyridine, Compound I-51 (0.0151 g, 0.039 mmol, 18.22% yield) as product.

¹H NMR (CDCl₃/400 MHz): δ (ppm) 8.81 (d, 2H), 7.26-7.30 (m, 1H), 7.19-7.20 (m, 1H), 7.11-7.14 (m, 1H), 7.04-7.09 (m, 2H), 5.46 (s, 2H), 4.75 (s, 2H), 3.62 (t, 2H), 2.85 (s, 3H), 2.75 (t, 2H). MS: 388.2 (M+1).

### Compound I-50

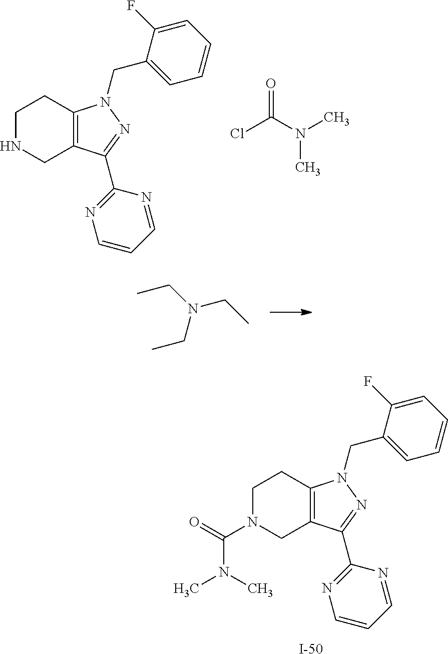

### Urea Formation:

To a stirring solution of 1-(2-fluorobenzyl)-3-(pyrimidin-2-yl)-4,5,6,7-tetrahydro-1H-pyrazolo[4,3-c]pyridine (0.068 g, 0.220 mmol) was added triethylamine (0.079 ml, 0.565 mmol) and dimethylcarbamic chloride (0.026 ml, 0.282 mmol). The reaction was stirred overnight at rt. Next day lcms showed completion of reaction and formation of desired product. The reaction mixture was concentrated in vacuo and purified by silica gel chromatography. The product, I-50, was a dark orange foamy hybrid between an oil and solid. ¹H NMR (CDCl₃/400 MHz): δ (ppm) 8.8 (d, 2H), 7.22-7.27 (m, 1H), 7.16 (t, 1H), 7.09-7.12 (m, 1H), 7.02-7.07 (m, 2H), 5.44 (s, 2H), 4.67 (s, 2H), 3.52 (t, 2H), 2.87 (s, 6H), 2.71 (t, 2H). MS: 268.1 (M+1).

### Compound I-52: 1-(1-(2-fluorobenzyl)-3-(pyrimidin-2-yl)-6,7-dihydro-1H-pyrazolo[4,3-c]pyridin-5(4H)-yl)ethanone

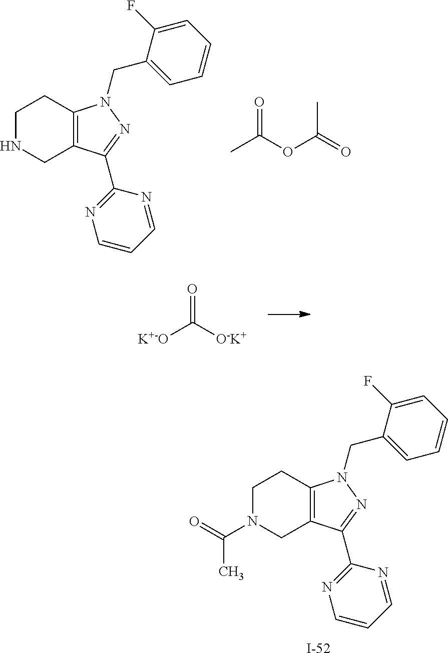

### Amide Formation:

To a stirring solution of 1-(2-fluorobenzyl)-3-(pyrimidin-2-yl)-4,5,6,7-tetrahydro-1H-pyrazolo[4,3-c]pyridine (0.06 g, 0.194 mmol) in DCM was added acetic anhydride (0.337 g, 3.30 mmol) and potassium carbonate (0.080 g, 0.582 mmol). The reaction was stirred overnight. The next day the reaction was concentrated and purified directly by silica gel chromatography. The final product was an orange solid 1-(1-(2-fluorobenzyl)-3-(pyrimidin-2-yl)-6,7-dihydro-1H-pyrazolo[4,3-c]pyridin-5(4H)-yl)ethanone, I-52 (0.0381 g, 0.108 mmol, 55.9% yield). ¹H NMR (CDCl₃/400 MHz): δ (ppm) 8.8 (br. s., 2H), 7.24-7.26 (m, 2H), 7.18-7.20 (m, 1H), 7.11-7.15 (m, 1H), 7.03-7.07 (m, 3H), 5.44 (s, 2H), 4.97 (s, 0.6H), 4.84 (s, 1.4H), 3.88 (t, 1.4H), 3.70 (t, 0.7H), 2.68 (t, 0.6H), 2.63 (t, 1.4H), 2.2 (s, 2H), 2.17 (s, 1H). MS: 352.2 (M+1). Rotamers account for unusual integrations.

### Compound I-54

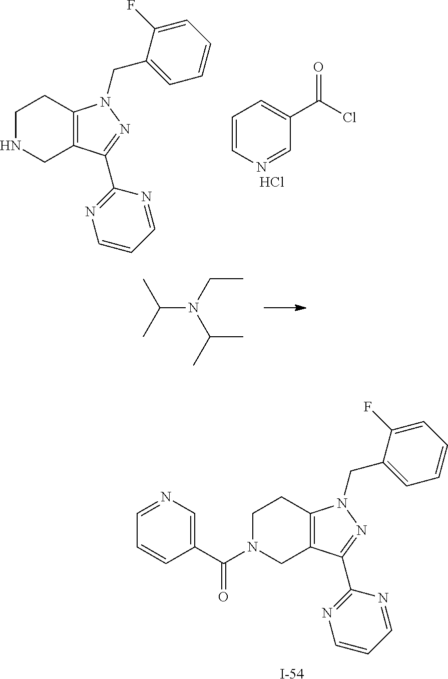

### Amide Formation:

Compound I-54 was synthesized according to the reaction scheme indicated above. ¹H NMR (CDCl₃/400 MHz): δ (ppm) 8.80 (d, 2H), 8.70 (s, 2H), 7.87 (s, 1H), 7.39-7.42 (m, 1H), 7.26-7.31 (m, 2H), 7.16-7.19 (m, 1H), 7.05-7.10 (m, 2H), 5.46 (s, 2H), 5.14 (br. s., 0.8H), 4.87 (br. s., 1.2H), 4.05 (br. s., 1.2H), 3.66 (br. s., 0.8H), 2.80 (br. s., 1.1H), 2.70 (br. s., 0.9H). MS: 415.2 (M+1). Rotamers account for unusual integrations.

### Compound I-55

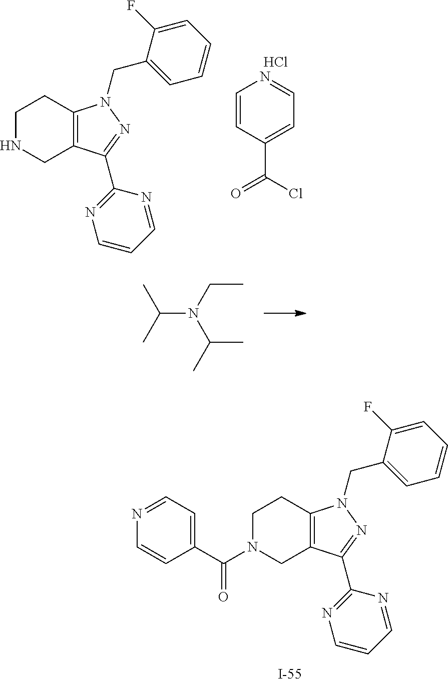

### Amide Formation:

Compound I-55 was prepared according to the reaction scheme indicated above. ¹H NMR (CDCl₃/400 MHz): δ (ppm) 8.82 (d, 1H), 8.74 (d, 2H), 8.67 (d, 1H), 7.54 (d, 1H), 7.41 (d, 1H), 7.26-7.30 (m, 1H), 7.19-7.21 (m, 2H), 7.07-7.13 (m, 2H), 5.45 (s, 2H), 5.14 (s, 0.8H), 4.75 (s, 1.2H), 4.06 (t, 1.2H), 3.59 (t, 0.8H), 2.81 (t, 1.2H), 2.66 (t, 0.8H). MS: 415.2 (M+1). Rotamers account for unusual integrations.

### Compound I-56

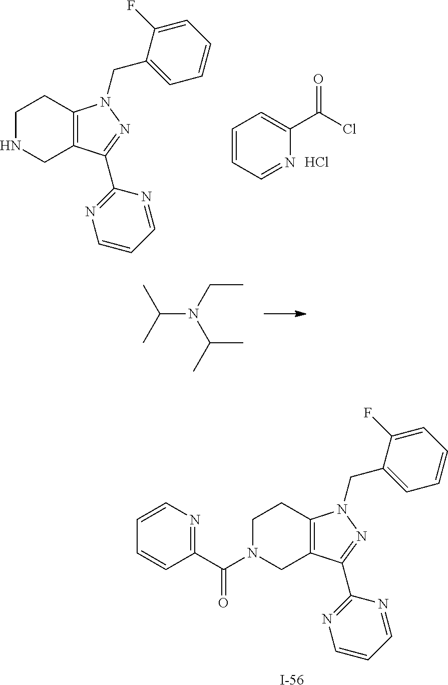

### Amide Formation:

Compound I-56 was synthesized according to the reaction scheme indicated above.

¹H NMR (CDCl₃/400 MHz): δ (ppm) 8.83 (d, 1H), 8.69 (d, 1H), 8.64 (d, 0.5H), 8.58 (d, 0.5H), 7.79-7.84 (m, 1H), 7.70 (d, 0.5H), 7.66 (d, 0.5H), 7.37 (t, 1H), 7.23-7.28 (m, 1H), 7.14-7.19 (m, 2H), 7.05-7.09 (m, 2H), 5.46 (s, 1H), 5.45 (s, 1H), 5.17 (s, 1H), 4.97 (s, 1H), 4.1 (t, 1H), 3.80 (t, 1H), 2.80 (t, 2H). MS: 415.2 (M+1). Rotamers account for unusual integrations.

### Compound I-57

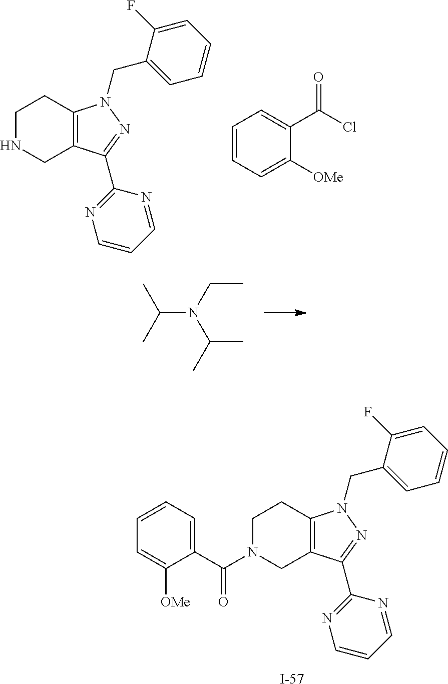

### Amide Formation:

Compound I-57 was synthesized according to the reaction scheme indicated above.

¹H NMR (CDCl₃/400 MHz): δ (ppm) 8.84 (d, 1H), 8.67 (d, 1H), 6.85-7.60 (m, 9H), 5.47 (s, 1H), 5.45 (s, 1H), 5.18 (dd, 1H), 4.65 (dd, 1H), 3.72 (s, 2H), 3.64 (s, 1H), 3.41-3.62 (m, 2H), 2.51-2.78 (m, 2H). MS not applicable for this compound. Rotamers account for unusual integrations.

### Compound I-58

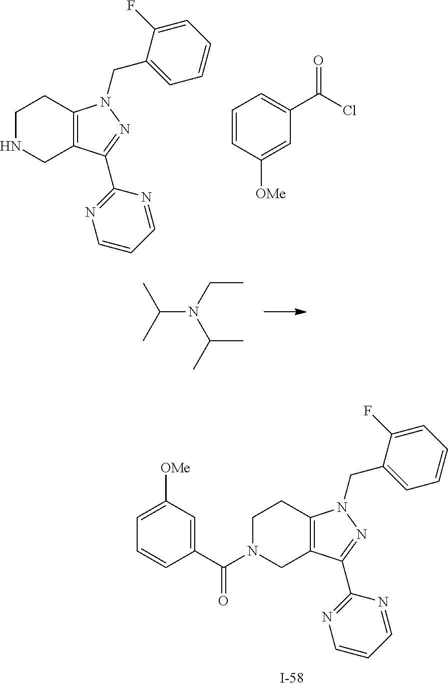

### Amide Formation:

Compound I-58 was synthesized according to the reaction scheme indicated above.

¹H NMR (CDCl₃/400 MHz): δ (ppm) 8.82 (s, 1H), 8.69 (s, 1H), 7.26-7.30 (m, 1H), 7.12-7.18 (m, 2H), 7.04-7.09 (m, 3H), 6.95-6.99 (m, 2H), 5.46 (s, 2H), 5.12 (s, 1H), 4.86 (s, 1H), 4.03 (s, 1H), 3.80 (s, 3H), 3.65 (s, 1H), 2.77 (s, 1H), 2.64 (s, 1H). MS not applicable for this compound. Rotamers account for unusual integrations.

### Compound I-63

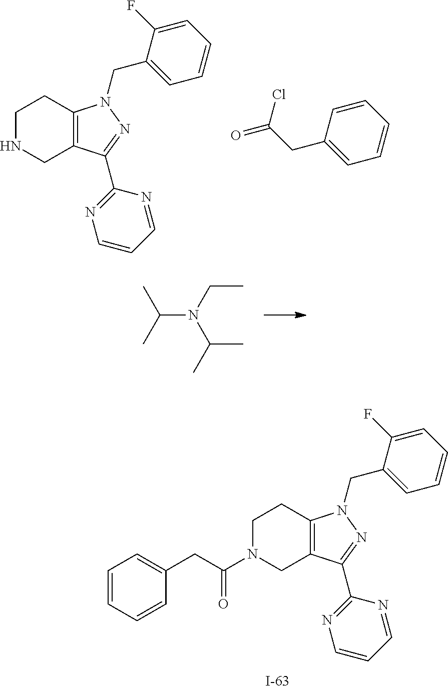

### Amide Formation:

Compound I-63 was synthesized according to the reaction scheme indicated above.

¹H NMR (CDCl₃/400 MHz): δ (ppm) 8.77-8.79 (m, 2H), 7.32-7.39 (m, 1H), 7.22-7.29 (m, 3H), 7.15-7.21 (m, 3H), 7.04-7.06 (m, 3H), 5.42 (s, 1.5H), 5.35 (s, 1H), 5.0 (s, 1H), 4.87 (s, 1.5H), 3.89 (t, 1.5H), 3.83 (m, 2H), 3.62-3.66 (m, 2H), 2.63 (t, 1.5H), 2.29 (t, 1H). MS: 428.2 (M+1). Rotamers account for unusual integrations.

### Example 7

### General Procedure E

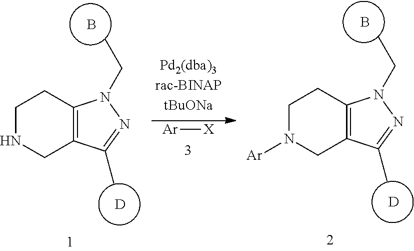

In the reaction scheme for General Procedure E shown above, Ar stands for an aryl or heteroaryl ring, X stands for halogen, wherein the halogen is Br or I, rac-BINAP stands for rac-2,2′-bis(diphenylphosphino)-1,1′-binaphthyl, and Pd₂(dba)₃ stands for tris(dibenzylideneacetone)dipalladium(0).

Cross Coupling:

To a solid mixture of pyrazole 1, rac-2,2′-bis(diphenylphosphino)-1,1′-binaphthyl (0.09 eq), tris(dibenzylideneacetone)dipalladium(0) (0.04 eq) and sodium tert-butoxide (1.4 eq) was added toluene. Aryl halide 3 (bromide or iodide, 1.1 eq) was then added to the reaction mixture. The resulting suspension was heated at 85° C. until completion by LC/MS analysis. Once complete, the reaction was poured into 1N NaOH solution and extracted two times with EtOAc. The organic portions were then combined, washed with brine, dried (Na₂SO₄), filtered, and concentrated. The crude material was purified using SiO₂ chromatography and an appropriate gradient (ethyl acetate/hexanes or DCM/methanol) to give compound 2, as a solid or liquid.

The following compounds were prepared according to the General Procedure E depicted above using the appropriate pyrazole 1 and aryl halide 3:

### Compound I-59: 1-(2-fluorobenzyl)-5-phenyl-3-(pyrimidin-5-yl)-4,5,6,7-tetrahydro-1H-pyrazolo[4,3-c]pyridine

¹H NMR: δ 8.82 (d, 2H), 7.27 (m, 3H), 7.17 (t, 1H), 7.04 (m, 5H), 6.85 (t, 1H), 5.46 (s, 2H), 4.65 (s, 2H), 3.62 (app. t, 2H), 2.73 (app. t, 2H)

MS: 386.2 (M+1)

### Compound I-60:1-(2-fluorobenzyl)-5-(pyridine-3-yl)-3-(pyrimidin-5-yl)-4,5,6,7-tetrahydro-1H-pyrazolo[4,3-c]pyridine

¹H NMR: δ 8.83 (d, 2H), 8.43 (d, 1H), 8.08 (d, 1H), 7.26 (m, 2H), 7.19 (m, 2H), 7.06 (m, 3H), 5.46 (s, 2H), 4.68 (s, 2H), 3.66 (app. t, 2H), 2.75 (app. t, 2H)

MS: 387.2 (M+1)

### Compound I-61: 1-(2-fluorobenzyl)-5-(pyridine-4-yl)-3-(pyrimidin-5-yl)-4,5,6,7-tetrahydro-1H-pyrazolo[4,3-c]pyridine

¹H NMR: δ 8.84 (d, 2H), 8.27 (d, 2H), 7.25 (m, 1H), 7.20 (t, 1H), 7.09 (m, 3H), 6.78 (d, 2H), 5.46 (s, 2H), 4.76 (s, 2H), 3.74 (app. t, 2H), 2.75 (app. t, 2H)

MS: 387.2 (M+1)

### Example 8

### General Procedure F

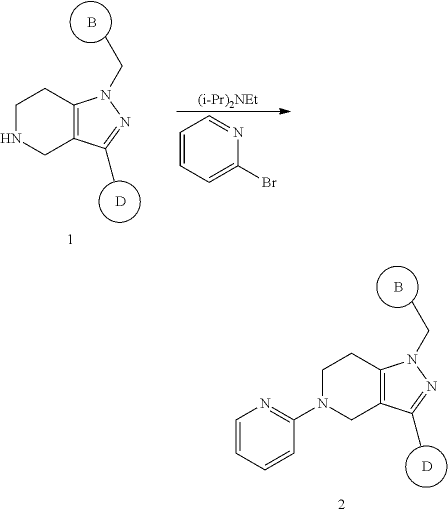

General procedure F can be used to prepare compounds of Formula I, wherein ring A is an azine and J^{A} is a pyridyl ring. In the reaction scheme for General Procedure F shown above, (i-Pr)₂NEt represents N-ethyl-N-isopropyl-2-propanamide. Compound 2 is the compound of Formula I prepared by the General Procedure F.

Aromatic Substitution:

To a suspension of pyrazole 1 in 2-bromopyridine (ca. 30 eq) as solvent was added N-ethyl-N-isopropyl-2-propanamine (2.0 eq). The reaction mixture was heated at 120° C. until complete (using TLC and LC/MS analysis). Once completed, the reaction was diluted with water and extracted three times with EtOAc. The organic portions were then combined, dried (Na₂SO₄), filtered, and concentrated. The crude material was purified using SiO₂ chromatography and an appropriate gradient (ethyl acetate/hexanes or DCM/methanol) to give compound 2, as a solid or liquid.

Compound I-62 was prepared as compound 2 according to the General Procedure F shown above using the corresponding pyrazole 1.

### Compound I-62: 1-(2-fluorobenzyl)-5-(pyridine-2-yl)-3-(pyrimidin-5-yl)-4,5,6,7-tetrahydro-1H-pyrazolo[4,3-c]pyridine

¹H NMR: δ 8.84 (d, 2H), 8.18 (m, 1H), 7.49 (m, 1H), 7.25 (m, 1H), 7.18 (t, 1H), 7.05 (m, 3H), 6.79 (d, 1H), 6.60 (m, 1H), 5.47 (s, 2H), 4.85 (s, 2H), 4.05 (app. t, 2H), 2.73 (app. t, 2H) MS: 387.2 (M+1)

### Example 9

### Biological Activity Measurement

sGC-HEK-cGMP Assay

Human embryonic kidney cells (HEK293), endogenously expressing soluble guanylate cyclase (sGC), were used to evaluate the activity of test compounds. Compounds stimulating the sGC receptor should cause an increase in the intracellular concentration of cGMP. HEK 293 cells were seeded in Dulbecco's Modification of Eagle's Medium supplemented with fetal bovine serum (10% final) and L-glutamine (2 mM final) in a 2004 volume at a density of 1×10⁵ cells/well in a poly-D-lysine coated 96 well flat bottom plate and grown overnight at 37° C. Medium was aspirated and cells were washed with 1× Hank's Buffered Saline Salt Solution (2004). Cells were then incubated for 15 minutes at 37° C. with 0.5 mM 3-isobutyl-1-methylxanthine (2004). Test article was then added to the assay mixture (2 μL) and incubated at 37° C. for 10 minutes. After the 10 minute incubation, the assay mixture was aspirated and 0.1M HCl (2004) was added to the cells. The plate was incubated at 4° C. for 30 minutes in the 0.1M HCl to stop the reaction and lyse the cells. The plates were then centrifuged at 1,200 g for 5 minutes at room temperature. Supernatants were collected and transferred to a new flat bottom 96 well plate for analysis. Vehicle controls were carried out using DMSO (1%). A known sGC stimulator, BAY 41-2272, was used as the positive control.

Samples were diluted with an equal volume of 1 M Ammonium Acetate (pH 7) to neutralize samples for better chromatography. A 2×cGMP standard curve was prepared in 0.1 M HCl and then diluted with an equal volume of 1 M Ammonium Acetate, with the following final concentrations in nM: 1024, 512, 256, 128, 64, 32, 16, 8, 4, 2, 1.

cGMP concentrations were determined from each sample using the LC/MS conditions (Table 2 below) and calculated standard curve. EC50 values were calculated from concentration-response curves generated with GraphPad Prism Software.

TABLE 2
(LC/MS experimental conditions)

| MS: | Thermo Quantum or Waters LCMS | | | | |
|---|---|---|---|---|---|
| Ion Mode: | ESI⁺ | | | | |
| Scan Type: | MRM | | | | |
| | | Dwell | Collision | | Retention |
| | | Time | Energy | Tube | Time |
| Compound: | Transition | (msec) | (V) | Lens | (min) |
| cGMP | 346 > 152 | 100 | 28 | 139 | 1.0 |

| HPLC: | Agilent Technologies 1200 Series with CTC Analytics | | |
|---|---|---|---|
| | HTS PAL | | |
| Column: | Thermo Hypersil Gold 2.1 × 50 mm 5 micron particle size | | |
| Flow Rate: | 400 uL/min | | |
| Column | RT | | |
| Temperature: | | | |
| Autosampler | 6° C. | | |
| Temperature: | | | |
| Injection | 20 uL | | |
| Volume: | | | |
| Mobile | A = 98:2 Water:Acetonitrile + 0.1% Formic Acid | | |
| Phases: | B = 2:98 Water:Acetonitrile + 0.1% Formic Acid | | |
| Gradient: | Time (min) | % A | % B |
| | 0 | 100 | 0 |
| | 0.3 | 30 | 70 |
| | 2.00 | 30 | 70 |
| | 2.01 | 100 | 0 |
| | 4 | 100 | 0 |

The biological activities of some of the compounds according to Formula I determined with the sGC-HEK assay are summarized in Table 3 below.

TABLE 3

| | Increase in cGMP | Increase in cGMP |
|---|---|---|
| Compound | Concentration Tested at | Concentration Tested |
| No. | 10 μM in HEK Assay* | at 30 μM in HEK Assay* |
| I-1 | D | D |
| I-2 | C | D |
| I-3 | D | E |
| I-4 | A | A |
| I-5 | B | C |
| I-6 | A | A |
| I-7 | C | D |
| I-8 | C | D |
| I-9 | A | B |
| I-10 | B | B |
| I-11 | A | A |
| I-12 | A | A |
| I-13 | A | A |
| I-15 | C | C |
| I-16 | A | B |
| I-17 | B | A |
| I-18 | B | C |
| I-19 | A | A |
| I-20 | A | A |
| I-21 | A | A |
| I-22 | D | E |
| I-23 | C | C |
| I-24 | A | A |
| I-25 | A | B |
| I-26 | A | B |
| I-27 | A | A |
| I-28 | B | C |
| I-29 | A | B |
| I-30 | A | A |
| I-31 | A | A |
| I-32 | A | A |
| I-33 | A | A |
| I-34 | A | A |
| I-35 | A | A |
| I-36 | C | B |
| I-37 | C | B |
| I-47 | A | A |
| I-48 | A | A |
| I-49 | A | A |
| I-50 | Not Determined | A |
| I-51 | A | A |
| I-52 | A | A |
| I-53 | A | A |
| I-54 | A | A |
| I-55 | A | A |
| I-56 | A | A |
| I-57 | B | C |
| I-58 | A | Not Determined |
| I-59 | A | A |
| I-60 | A | A |
| I-61 | A | A |
| I-62 | A | A |

*Letter codes for the increase:
A represents no increase or an increase ranging from more than 0 fold to less than 5 fold;
B represents an increase ranging from 5 fold to less than 10 fold;
C represents an increase ranging from 10 fold to less than 20 fold;
D represents an increase ranging from 20 fold to less than 50 fold; and
E represents an increase ranging from 50 fold to less than 100 fold.

### Example 10

### Biological Activity Measurement

### Thoracic Aortic Rings Assay

Thoracic aortic rings were dissected from anesthetized (isoflurane) male Sprague-Dawley rats weighing 275-299 g. Tissues were immediately transferred to ice-cold Krebs-Henseleit solution, which had been aerated with 95% O₂ and 5% CO₂ for 30 minutes. Following removal of connective tissue, aortic sections were cut into 4 rings (˜2 mm each) and suspended on 2 L-shaped hooks, with one hook fixed at the bottom of the tissue bath (Schuler Organ Bath, Harvard Apparatus) and the other connected to a force transducer (F30 Force Transducer, Harvard Apparatus). Baths contained Krebs Henseleit solution (10 mL) heated to 37° C. and aerated with 95% O₂ and 5% CO₂. Rings were brought to an initial tension of 0.3-0.5 g and gradually raised to a resting tension of 1.0 g over 60 minutes. Rings were rinsed with Krebs Henseleit solution (heated to 37° C. and aerated with 95% O₂ and 5% CO₂) at 15 minute intervals until a stable baseline was obtained. Rings were considered to be stable after a resting tension of 1.0 g was maintained (for approximately 10 minutes) without need for adjustment. Rings were then contracted with 100 ng/mL phenylephrine by adding 100 uL of a 10 mg/mL phenylephrine stock solution. Tissues achieving a stable contraction were then treated in a cumulative, dose dependent manner with test compounds prepared in dimethylsulfoxide (DMSO). In some cases, tissues were rinsed three times over a 5 minute period with Krebs-Heinseleit's solution (heated to 37° C. and aerated with 95% O₂ and 5% CO₂), allowed to stabilize at baseline, and then used for characterization of other test articles or DMSO effects. All data were collected using the HSE-ACAD software provided by Harvard Apparatus. Percent relaxation effects were calculated in Microsoft Excel using the recorded tension value of 100 ng/mL phenylephrine treatment as 0% inhibition and treatment with 100 μM 3-isobutyl-1-methylxanthine as 100% inhibition. EC50 values were calculated from concentration-response curves generated with GraphPad Prism Software.

The biological data for some of the compounds of Formula I, in comparison with the prior art compound, BAY 41-2272, as the reference compound, determined by the thoracic aorta ring assay are presented in Table 4 below.

TABLE 4
Thoracic aortic ring assay activity.

| Compound Tested | EC50 (μM) |
|---|---|
| Reference Cpd. | 0.34-0.95 |
| I-22 | 2 |
| I-1 | 1.3 |
| I-3 | 1.2 |
| I-37 | 1.2 |

A number of embodiments have been described. Nevertheless, it will be understood that various modifications may be made without departing from the spirit and scope of the invention.

1. A compound according to Formula I, or a pharmaceutically acceptable salt thereof, 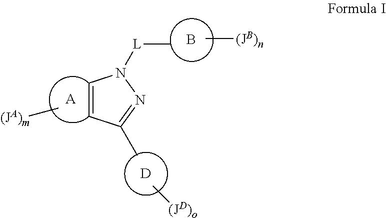
wherein:
ring A is selected from a 5 to 10-membered cycloaliphatic ring or a 5 to 10-membered non-aromatic heterocycle; wherein said heterocycle contains from 1 to 3 heteroatoms independently selected from O or S;
m is an integer selected from 0 to 3;
if J^{A} is a substituent on a ring carbon atom, J^{A} is independently selected from halogen, —CN, —NO₂, a C₁₋₆ aliphatic, —OR^{A}, —SR^{A}, —COR^{A}, —C(O)OR^{A}, —C(O)N(R^{A})₂, —N(R^{A})₂, —N(R^{A})C(O)R^{a}, —N(R^{A})C(O)OR^{a}, —SO₂R^{A}, —SO₂N(R^{A})₂, —N(R^{A})SO₂R^{a}, —N(R^{A})SO₂N(R^{a})₂, a C₃₋₈ cycloaliphatic ring, a 4 to 8-membered heterocyclic ring, a 5 to 6-membered heteroaryl ring or an oxo group; wherein each said C₁₋₆ aliphatic, said C₃₋₈ cycloaliphatic ring, said 4 to 8-membered heterocyclic ring and said 5 to 6-membered heteroaryl ring is independently substituted with from 0 to 3 substituents selected from halogen, —OH, —O(C₁₋₄ alkyl), —O(C₁₋₄ haloalkyl), —NH₂, —N(C₁₋₄ alkyl)₂, —NH(C₁₋₄ alkyl), —COOH, —NO₂, —CN or an oxo group;
each R^{A} is independently selected from hydrogen, C₁₋₆ aliphatic, a C₃₋₈ cycloaliphatic ring, a 4 to 8-membered heterocyclic ring, phenyl or a 5 to 6-membered heteroaryl ring; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted with from 0 to 3 instances of R¹;
each R^{a} is independently selected from hydrogen, C₁₋₆ aliphatic, a C₃₋₈ cycloaliphatic ring, a 4 to 8-membered heterocyclic ring, phenyl or a 5 to 6-membered heteroaryl ring; wherein each said heterocylic ring and said heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl rings is independently substituted by from 0 to 3 instances of R¹;
each R¹ is independently selected from halogen, —CN, C₁₋₄ alkyl, C₁₋₄ haloalkyl, —OR², —SR², —COR², —C(O)OR², —C(O)N(R²)₂, —N(R²)C(O)R², —N(R²)₂, —SO₂R², —SO₂N(R²)₂, —N(R)SO₂R, phenyl or an oxo group, wherein said phenyl group is optionally substituted with from 0 to 3 substituents independently selected from halogen, hydroxy, —NH₂, —N(C₁₋₄ alkyl)H, —N(C₁₋₄ alkyl)₂, —NO₂, —CN, C₁₋₄ alkyl, C₁₋₄ haloalkyl, C₁₋₄ alkoxy or —O(C₁₋₄ haloalkyl);
each R² is independently selected from hydrogen, a C₁₋₄ alkyl, phenyl, benzyl or a C₃₋₈ cycloalkyl group, each of said C₁₋₄ alkyl, said phenyl, said benzyl and said C₃₋₈ cycloalkyl group independently substituted by from 0 to 3 instances of halogen; or alternatively two R² groups attached to the same nitrogen atom, together with said nitrogen atom may form a 5 to 8-membered heterocyclic ring or a 5-membered heteroaryl ring; each said 5 to 8-membered heterocyclic ring and said 5-membered heteroaryl ring containing 1 or 2 additional heteroatoms independently selected from N, O or S;
if J^{A} is a substituent on a ring sulfur atom, when present, J^{A} is oxo;
or, alternatively, two J^{A} groups attached to two non-vicinal ring atoms of ring A, together with said non-vicinal atoms, form a C₅₋₈ carbocyclic ring or a 5 to 8-membered heterocyclic ring with said two J^{A} groups forming a bridge for ring A between the two non-vincinal ring atoms; wherein said 5 to 8-membered heterocyclic ring contains 1 or 2 heteroatoms independently selected from S or O; and wherein said C₅₋₈ carbocyclic ring or 5 to 8-membered heterocyclic ring formed by said two J^{A} groups is optionally and independently substituted with from 0 to 2 substituents selected from halogen, hydroxy, —NH₂, —N(C₁₋₄ alkyl)H, —N(C₁₋₄ alkyl)₂, —NO₂, —CN, C₁₋₄ alkyl, C₁₋₄ alkoxy, C₁₋₄ haloalkyl or C₁₋₄ haloalkoxy;
L is a methylene linker, independently substituted by from 0 to 2 substituents selected from halogen or C₁₋₆ alkyl, wherein when two substituents on the methylene linker are C₁ alkyl groups, the two C₁ alkyl groups together with the carbon atom to which the two C₁ alkyl groups are attached may form a cyclopropyl ring; wherein each said C₁₋₆ alkyl and cyclopropyl is optionally and independently substituted by from 0 to 3 instances of halogen;
ring B is selected from a monocyclic or bicyclic 6 to 10-membered aryl or a 6 to 10-membered heteroaryl; wherein said 6 to 10-membered heteroaryl contains from 1 to 4 heteroatoms independently selected from N, O or S;
n is an integer selected from 0 to 3;
if J^{B} is a substituent on a ring carbon atom, J^{B} is independently selected from halogen, —CN, —NO₂, a C₁₋₆ aliphatic, —OR^{B}, —SR^{B}, —COR^{E}, —C(O)OR^{B}, —C(O)N(R^{B})₂, —N(R^{B})₂, —N(R^{B})C(O)R^{b}, —N(R^{B})C(O)OR^{b}, —SO₂R^{B}, —SO₂N(R^{B})₂, —N(R^{B})SO₂R^{b}, —N(R^{B})SO₂N(R^{b})₂, a C₃₋₈ cycloaliphatic group, a 4 to 8-membered heterocyclic group, a 5 to 6-membered heteroaryl group or an oxo group; wherein each said C₁₋₆ aliphatic, said C₃₋₈ cycloaliphatic group, said 4 to 8-membered heterocyclic group and said 5 to 6-membered heteroaryl group is independently substituted with from 0 to 3 substituents selected from halogen, —OH, C₁₋₄ alkyl, C₁₋₄ haloalkyl, —O(C₁₋₄ alkyl), —O(C₁₋₄ haloalkyl), —NH₂, —N(C₁₋₄ alkyl)₂, —NH(C₁₋₄ alkyl), —COOH, —CN, —NO₂ or oxo;
if J^{B} is a substituent on a ring nitrogen atom, when present, J^{B} is independently selected from —C(O)R^{B}, —C(O)OR^{B}, —C(O)N(R^{B})₂, —SO₂R^{B}, —SO₂N(R^{B})₂, a C₁₋₆ aliphatic, a —(C₁₋₆ aliphatic)-R^{B}, a C₃₋₈ cycloaliphatic ring, a 4 to 8-membered heterocyclic ring, or a 5 to 6-membered heteroaryl ring; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring and said 5 to 6-membered heteroaryl ring is independently substituted with from 0 to 3 instances of R³;
or, alternatively, two J^{B} groups attached to two vicinal ring B atoms, taken together with said two vicinal ring B atoms, form a 5 to 7-membered heterocycle resulting in a fused ring B; wherein said 5 to 7-membered heterocycle contains from 1 to 2 heteroatoms independently selected from N, O or S; and wherein said 5 to 7-membered heterocycle is optionally substituted by from 0 to 3 substituents independently selected from halogen, hydroxy, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl);
each R^{B} is independently selected from hydrogen, a C₁₋₆ aliphatic, a C₃₋₈ cycloaliphatic ring, a 4 to 8-membered heterocyclic ring, phenyl or a 5 to 6-membered heteroaryl ring; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted with from 0 to 3 instances of R³;
each R^{b} is independently selected from hydrogen, a C₁₋₆ aliphatic, a C₃₋₈ cycloaliphatic ring, a 4 to 8-membered heterocyclic ring, phenyl or a 5 to 6-membered heteroaryl ring; wherein each said heterocylic ring and said heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted by from 0 to 3 instances of R³;
each R³ is independently selected from halogen, —CN, C₁₋₄ alkyl, C₁₋₄ haloalkyl, —OR⁴, —SR⁴, —COR⁴, —C(O)OR⁴, —C(O)N(R⁴)₂, —N(R⁴)C(O)R⁴, —N(R⁴)₂, —SO₂R⁴, —SO₂N(R⁴)₂, —N(R⁴)SO₂R⁴, phenyl or an oxo group, wherein each said phenyl group is optionally substituted with from 0 to 3 substituents independently selected from halogen, hydroxy, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl);
each R⁴ is independently selected from hydrogen, a C₁₋₄ alkyl, phenyl, benzyl or a C₃₋₈ cycloalkyl group, each of said C₁₋₄ alkyl, said phenyl, said benzyl and said cycloalkyl group independently substituted by from 0 to 3 instances of halogen; or alternatively two R⁴ groups attached to the same nitrogen atom, together with said nitrogen atom form a 5 to 8-membered heterocyclic ring or a 5-membered heteroaryl ring; each said 5 to 8-membered heterocyclic ring and said 5-membered heteroaryl ring containing 1 or 2 additional heteroatoms independently selected from N, O or S;
ring D is a 6-membered heteroaryl which contains from 1 to 3 instances of N;
o is an integer selected from 0 to 3;
if J^{D} is a substituent on a ring carbon atom, it is independently selected from halogen, —NO₂, oxo, —OR^{D}, —C(O)R^{D}, —C(O)OR^{D}, —C(O)N(R^{D})₂, —CN, —N(R^{D})₂, —N═NR^{D}, —N(R^{D})C(O)R^{d}, —N(R^{D})C(O)OR^{d}, —SO₂R^{D}, —SO₂N(R^{D})₂, —N(R^{D})SO₂R^{d}, C₁₋₆ aliphatic, —(C₁₋₆ aliphatic)-R^{D}, a C₃₋₈ cycloaliphatic ring, a 6 or 10-membered aryl ring, a 4 to 8-membered heterocyclic ring or a 5 to 6-membered heteroaryl; wherein each said 4 to 8-membered heterocylic ring and said 5 to 6-membered heteroaryl ring contains between 1 and 3 heteroatoms independently selected from O, N or S; and wherein each said C₁₋₆ aliphatic, said C₃₋₈ cycloaliphatic ring, said 6 or 10-membered aryl ring, said 4 to 8-membered heterocyclic ring and said 5 to 6-membered heteroaryl ring is independently substituted with from 0 to 3 instances of R⁵;
each R^{D} is independently selected from hydrogen, a C₁₋₆ aliphatic, a C₃₋₈ cycloaliphatic ring, a 4 to 8-membered heterocyclic ring, phenyl or a 5 to 6-membered heteroaryl ring; wherein each said 4 to 8-membered heterocylic and said 5 to 6-membered heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted with from 0 to 3 instances of R⁵;
each R^{d} is independently selected from hydrogen, a C₁₋₆ aliphatic, a C₃₋₈ cycloaliphatic ring, a 4 to 8-membered heterocyclic ring, phenyl or a 5 to 6-membered heteroaryl ring; wherein each said heterocylic ring and said heteroaryl ring contains between 1 and 3 heteroatoms independently selected from O, N or S; and wherein each said C₁₋₆ aliphatic, said C₃₋₈ cycloaliphatic ring, said 4 to 8-membered heterocyclic ring, said phenyl and said 5 to 6-membered heteroaryl ring is independently substituted by from 0 to 3 instances of R⁵;
each R⁵ is independently selected from halogen, —CN, C₁₋₄ alkyl, C₁₋₄ haloalkyl, —OR⁶, —SR⁶, —COR^{E}, —C(O)OR⁶, —C(O)N(R⁶)₂, —N(R⁶)C(O)R⁶, —N(R⁶)₂, —SO₂R⁶, —SO₂N(R⁶)₂, —N(R⁶)SO₂R⁶, phenyl or an oxo group, wherein each said phenyl group is optionally substituted with from 0 to 3 substituents independently selected from halogen, hydroxy, —NH₂, —NH(C₁₋₄ alkyl), —N(C₁₋₄ alkyl)₂, —NO₂, —CN, C₁₋₄ alkyl, C₁₋₄ haloalkyl, —O(C₁₋₄ alkyl) or —O(C₁₋₄ haloalkyl);
each R⁶ is independently selected from hydrogen, a C₁₋₄ alkyl, phenyl, benzyl or a C₃₋₈ cycloalkyl group, each of said C₁₋₄ alkyl, said phenyl, said benzyl and said cycloalkyl group independently substituted by from 0 to 3 instances of halogen; or alternatively two R⁶ groups attached to the same nitrogen atom, together with said nitrogen atom form a 5 to 8-membered heterocyclic ring or a 5-membered heteroaryl ring; each said 5 to 8-membered heterocyclic ring and said 5-membered heteroaryl ring containing 1 or 2 additional heteroatoms independently selected from N, O or S;
or, alternatively, two J^{D} groups attached to two vicinal ring D atoms, taken together with said two vicinal ring D atoms, form a 5 to 7-membered heterocycle resulting in a fused ring D wherein said 5 to 7-membered heterocycle contains from 1 to 3 heteroatoms independently selected from N, O or S; and wherein said 5 to 7-membered heterocycle is optionally and independently substituted by from 0 to 3 substituents selected from halogen, hydroxy, —NH₂, —NH(C₁₋₄ alkyl), —N(C₁₋₄ alkyl)₂, —CN, C₁₋₄ alkyl, C₁₋₄ haloalkyl, —O(C₁₋₄ alkyl) or —O(C₁₋₄ haloalkyl);
provided that the compound according to Formula I is not:

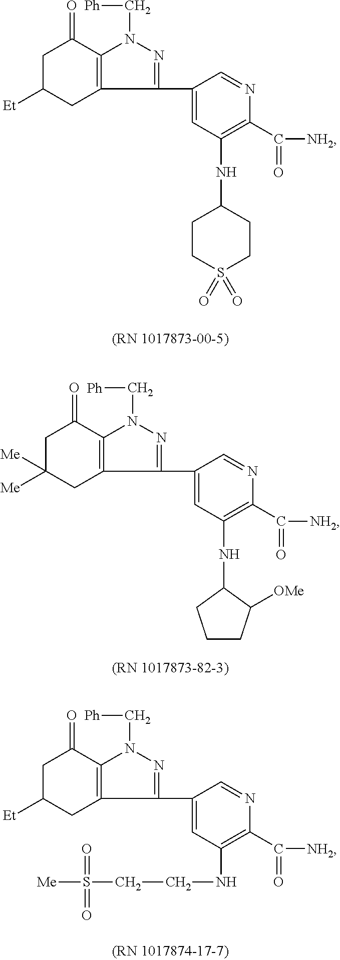 [SMILES: CCC1CC(=O)c2c(c(-c3cnc(C(N)=O)c(NC4CCS(=O)(=O)CC4)c3)nn2Cc2ccccc2)C1 ; COC1CCCC1Nc1cc(-c2nn(Cc3ccccc3)c3c2CC(C)(C)CC3=O)cnc1C(N)=O ; CCC1CC(=O)c2c(c(-c3cnc(C(N)=O)c(NCCS(C)(=O)=O)c3)nn2Cc2ccccc2)C1]

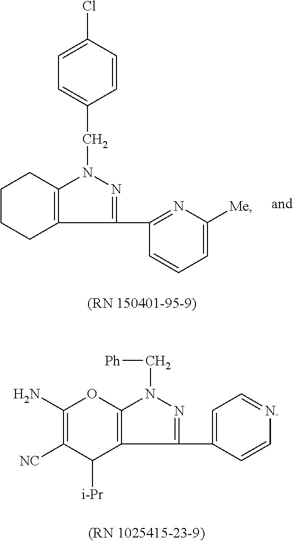 [SMILES: Cc1cccc(-c2nn(Cc3ccc(Cl)cc3)c3c2CCCC3)n1 ; CC(C)C1C(C#N)=C(N)Oc2c1c(-c1ccncc1)nn2Cc1ccccc1]

2. The compound of claim 1, wherein ring A is a 5 to 7-membered cycloaliphatic ring or 5 or 6-membered non-aromatic heterocycle, wherein the 5 or 6-membered non-aromatic heterocycle contains from 1 to 3 heteroatoms independently selected from O or S.

3. The compound of claim 2, wherein ring A is a 5 or 6-membered cycloaliphatic ring.

4. The compound of claim 3, wherein ring A is a 5-membered cycloaliphatic ring.

5. The compound of claim 3, wherein ring A is a 6-membered cycloaliphatic ring.

6. The compound of claim 1, wherein ring A is a 5 or 6-membered non-aromatic heterocycle.

7. The compound of claim 6, wherein ring A is a 6-membered non-aromatic heterocycle.

8. The compound of claim 7, wherein 1 ring A is a 6-membered non-aromatic heterocycle having 1 or 2 ring S heteroatoms.

9. The compound of claim 8, wherein ring A is a 6-membered non-aromatic heterocycle having one ring S heteroatom.

10. The compound of claim 9, wherein ring A is a 5-membered non-aromatic heterocycle having one ring S heteroatom.

11. The compound of any one of claims 1-10, wherein when J^{A} is a substituent on a ring carbon atom J^{A} is independently selected from halogen, C₁₋₆ aliphatic, oxo, —OR^{A}, —COR^{A}, —C(O)OR^{A}, —C(O)N(R^{A})₂, —CN, —N(R^{A})₂, —N(R^{A})C(O)R^{a}, —N(R^{A})C(O)OR^{a}, —SO₂R^{A}, —SO₂N(R^{A})₂ or —N(R^{A})SO₂N(R^{a})₂.

12. The compound of claim 11, wherein at least one J^{A} is a substituent on a ring carbon atom independently selected from halogen, C₁₋₆ aliphatic, oxo, —OR^{A}, —COR^{A}, —C(O)OR^{A}, —C(O)N(R^{A})₂, —CN, —N(R^{A})₂, —N(R^{A})C(O)OR^{a}, —N(R^{A})C(O)OR^{a}, —SO₂R^{A}, —SO₂N(R^{A})₂, —SO₂N(R^{A})₂ or —N(R^{A})SO₂N(R^{a})₂.

13. The compound of claim 12, wherein each J^{A} is independently selected from halogen or C₁₋₆ aliphatic groups.

14. The compound of claim 13, wherein each J^{A} is independently selected from C₁₋₆ aliphatic groups.

15. The compound of claim 14, wherein each J^{A} is methyl.

16. The compound of any one of claims 1-15, wherein m is 1 or 2.

17. The compound of claim 16, wherein m is 2.

18. The compound of any one of claims 1-10, wherein m is 0.

19. The compound of claim 13, wherein each J^{A} is independently selected from halogen.

20. The compound of claim 19, wherein the halogen is fluoro.

21. The compound of claim 16, wherein the at least one J^{A} is independently selected from oxo or methyl.

22. The compound of any one of claims 1-21, wherein ring B is phenyl, a bicyclic 10-membered aryl ring, a 6-membered heteroaryl ring or a bicyclic 9 or 10-membered heteroaryl ring.

23. The compound of claim 22, wherein ring B is a 6-membered heteroaryl ring.

24. The compound of claim 22, wherein ring B is phenyl.

25. The compound of claim 22, wherein ring B is substituted with 1 to 3 J^{B} substituents and wherein at least one of the J^{B} substituents is ortho to the attachment of L.

26. The compound of claim 23, wherein ring B is substituted with 1 to 3 J^{B} substituents and wherein at least one of the J^{B} substituents is ortho to the attachment of L.

27. The compound of claim 24, wherein ring B is substituted with 1 to 3 J^{B} substituents and wherein at least one of the J^{B} substituents is ortho to the attachment of L.

28. The compound of claim 23, wherein ring B is substituted with one J^{B} substituent ortho to the attachment of L.

29. The compound of claim 24, wherein ring B is substituted with one J^{B} substituent ortho to the attachment of L.

30. The compound of claim 26, wherein at least one of the 1 to 3 J^{B} substituents is a substituent on a ring carbon atom independently selected from halogen, C₁₋₆ aliphatic, —CN, —N(R^{B})₂ or —OR^{B}.

31. The compound of claim 30, wherein at least one of the 1 to 3 J^{B} substituents is a substituent on a ring carbon atom independently selected from halogen, —OR^{B} or —CN.

32. The compound of claim 31, wherein at least one of the 1 to 3 J^{B} substituents is a substituent on a ring carbon atom independently selected from halogen atoms.

33. The compound of claim 32, wherein at least one of the 1 to 3 J^{B} substituents is a fluorine or chlorine atom attached to a ring carbon atom.

34. The compound of claim 33, wherein at least one of the 1 to 3 J^{B} substituents is a fluorine atom attached to a ring carbon atom.

35. The compound of claim 27, wherein at least one of the 1 to 3 J^{B} substituents is a substituent independently selected from halogen, C₁₋₆ aliphatic, —CN, —N(R^{B})₂ or —OR^{B}.

36. The compound of claim 35, wherein at least one of the 1 to 3 J^{B} substituents is a substituent independently selected from halogen, C₁₋₆ aliphatic or —CN.

37. The compound of claim 36, wherein at least one of the 1 to 3 J^{B} substituents is a substituent independently selected from halogen atoms.

38. The compound of claim 37, wherein at least one of the 1 to 3 J^{B} substituents is a fluorine or chlorine atom.

39. The compound of claim 38, wherein at least one of the 1 to 3 J^{B} substituents is a fluorine atom.

40. The compound of claim 27, wherein there is one J^{B} substituent attached to ring B, the J^{B} substituent is ortho to the attachment of L and the J^{B} substituent is selected from halogen, C₁₋₆ aliphatic, —CN, —N(R^{B})₂ or —OR^{B}.

41. The compound of claim 40, wherein the J^{B} substituent is selected from halogen, C₁₋₆ aliphatic or —CN.

42. The compound of claim 41, wherein the J^{B} substituent is halogen.

43. The compound of claim 42, wherein the J^{B} substituent is a fluorine or chlorine atom.

44. The compound of claim 43, wherein the J^{B} substituent is a fluorine atom.

45. The compound of claim 23, wherein ring B is pyridinyl.

46. The compound of claim 45, wherein ring B is pyridin-3-yl.

47. The compound of claim 23, wherein ring B is pyrimidinyl.

48. The compound of claim 47, wherein ring B is pyrimidin-5-yl.

49. The compound of claim 1, wherein ring D is pyridinyl, pyrimidinyl or 1,3,5-triazinyl.

50. The compound of claim 48, wherein ring D is 1,3,5-triazin-2-yl.

51. The compound of claim 49, wherein ring D is pyridinyl.

52. The compound of claim 49, wherein ring D is pyrimidinyl.

53. The compound of claim 52, wherein ring D is pyrimidin-2-yl.

54. The compound of claim 52, wherein ring D is pyrimidin-5-yl.

55. The compound of any one of claims 1-54, wherein J^{D} is a substituent on a ring carbon atom independently selected from halogen, an oxo group, —C(O)R^{D}, —CN, —N(R^{D})₂, —N═N—R^{D}, —N(R^{D})C(O)R^{d}, —N(R^{D})C(O)OR^{d}, —SO₂R^{D}, —SO₂N(R^{D})₂, —N(R^{D})SO₂R^{d}, C₁₋₆ aliphatic, a —(C₁₋₆ aliphatic)-R^{D}, a 6 or 10-membered aryl ring, a 4 to 8-membered heterocyclic ring or a 5 to 6-membered heteroaryl ring, wherein each said 4 to 8-membered heterocylic ring and said 5 to 6-membered heteroaryl ring contains between 1 and 3 heteroatoms independently selected from O, N or S; and wherein each said C₁₋₆ aliphatic, said 6 or 10-membered aryl ring, said 4 to 8-membered heterocyclic ring and said 5 to 6-membered heteroaryl ring is independently substituted with from 0 to 3 instances of R⁵.

56. The compound of claim 55, wherein J^{D} is a substituent on a ring carbon atom independently selected from —N(R^{D})₂, —N═N—R^{D}, —N(R^{D})C(O)R^{d}, —N(R^{D})C(O)OR^{d}, a 6 or 10-membered aryl ring, a 4 to 8-membered heterocyclic ring or a 5 or 6-membered heteroaryl ring.

57. The compound of claim 56, wherein J^{D} is a substituent on a ring carbon atom independently selected from —N(R^{D})₂, —N═N—R^{D}, —N(R^{D})C(O)R^{d}, —N(R^{D})C(O)OR^{d}, phenyl, a 5 or 6-membered heterocyclic ring or a 5 or 6-membered heteroaryl ring, wherein each said phenyl, said 5 or 6-membered heterocyclic ring and said 5 or 6-membered heteroaryl ring is independently substituted with from 0 to 3 instances of R⁵.

58. The compound of claim 57, wherein J^{D} is a substituent on a ring carbon atom independently selected from —N(R^{D})₂, —N(R^{D})C(O)R^{d} or —N(R^{D})C(O)OR^{d}.

59. The compound of claim 58, wherein o is 1 or 2.

60. The compound of claim 59, wherein o is 1.

61. The compound of claim 59, wherein o is 2.

62. The compound of claim 60, wherein J^{D} is —NH₂.

63. The compound of claim 61, wherein one of the J^{D} substituents is —NH₂.

64. The compound of claim 63, wherein both J^{D} substituents are —NH₂.

65. The compound of any one of claims 1-54, wherein o is 0.

66. The compound of claim 4, wherein ring B is phenyl and ring D is pyrimidyl.

67. The compound of claim 66, wherein ring B is phenyl substituted with a fluorine atom ortho or meta to the attachment of L.

68. The compound of claim 67, wherein ring B is phenyl substituted with a fluorine atom ortho to the attachment of L.

69. The compound of claim 68, wherein ring D is pyrimidin-2-yl.

70. The compound of claim 5, wherein ring B is phenyl and ring D is pyrimidyl.

71. The compound of claim 70, wherein ring B is phenyl substituted with a fluorine atom ortho or meta to the attachment of L.

72. The compound of claim 71, wherein ring B is phenyl substituted with a fluorine atom ortho to the attachment of L.

73. The compound of claim 72, wherein ring D is pyrimidin-2-yl.

74. The compound of claim 1, selected from Compound Nos. I-1 to I-37 and I-41 to I-49 listed in Table 1.

75. The compound of claim 1 with the further proviso that the compound is not a derivative or pharmaceutically acceptable salt of the compound represented by CAS Registry Number: RN 1017873-00-5, RN 1017873-82-3, RN 1017874-17-7, RN 150401-95-9 or RN 1025415-23-9, wherein a H atom of the compound represented by said CAS Registry Number is replaced with a methyl or ethyl group, or a methyl group of the compound represented by said CAS Registry Number is replaced with a H atom.

76. A method of treating a disease, health condition or disorder in a subject, comprising administering a therapeutically effective amount of the compound of claim 1 to the subject in need of the treatment, wherein the disease, health condition or disorder is
(a) a peripheral or cardiac vascular disorder or health condition selected from: pulmonary hypertension, pulmonary arterial hypertension, and associated pulmonary vascular remodeling, localized pulmonary thrombosis, right heart hypertophy, pulmonary hypertonia, primary pulmonary hypertension, secondary pulmonary hypertension, familial pulmonary hypertension, sporadic pulmonary hypertension, pre-capillary pulmonary hypertension, idiopathic pulmonary hypertension, thrombotic pulmonary arteriopathy, plexogenic pulmonary arteriopathy; pulmonary hypertension associated with or related to: left ventricular dysfunction, hypoxemia, mitral valve disease, constrictive pericarditis, aortic stenosis, cardiomyopathy, mediastinal fibrosis, pulmonary fibrosis, anomalous pulmonary venous drainage, pulmonary venooclusive disease, pulmonary vasculitis, collagen vascular disease, congenital heart disease, pulmonary venous hypertension, interestitial lung disease, sleep-disordered breathing, apnea, alveolar hypoventilation disorders, chronic exposure to high altitude, neonatal lung disease, alveolar-capillary dysplasia, sickle cell disease, other coagulation disorders, chronic thromboemboli, pulmonary embolism, connective tissue disease, lupus, schitosomiasis, sarcoidosis, chronic obstructive pulmonary disease, emphysema, chronic bronchitis, pulmonary capillary hemangiomatosis; histiocytosis X, lymphangiomatosis and compressed pulmonary vessels;
(b) a health disorder related to high blood pressure and decreased coronary blood flow selected from:
increased acute and chronic coronary blood pressure, arterial hypertension, vascular disorder resulting from heart disease, stroke, cerebral ischemia, or renal failure, congestive heart failure, thromboembolic disorders, ischemias, myocardial infarction, stroke, transient ischemic attacks, stable or unstable angina pectoris, arrythmias, diastolic dysfunction, coronary insufficiency;
(c) atherosclerosis, restenosis, percutaneous transluminal coronary angioplasties or inflammation;
(d) liver cirrhosis, hepatic fibrosis, hepatic stellate cell activation, hepatic fibrous collagen and total collagen accumulation, liver disease of necro-inflammatory and/or of immunological origin; or
(e) a urogenital system disorder selected from renal fibrosis, renal failure resulting from chronic kidney diseases or insufficiently, prostate hypertrophy, erectile dysfunction, female sexual dysfunction and incontinence.

77. The method of claim 76, wherein the disease, health condition or disorder is
(a) a peripheral or cardiac vascular disorder or health condition selected from: pulmonary hypertension, pulmonary arterial hypertension, and associated pulmonary vascular remodeling, localized pulmonary thrombosis, right heart hypertophy, pulmonary hypertonia, primary pulmonary hypertension, secondary pulmonary hypertension, familial pulmonary hypertension, sporadic pulmonary hypertension, pre-capillary pulmonary hypertension, idiopathic pulmonary hypertension, thrombotic pulmonary arteriopathy, plexogenic pulmonary arteriopathy; pulmonary hypertension associated with or related to: left ventricular dysfunction, hypoxemia, mitral valve disease, constrictive pericarditis, aortic stenosis, cardiomyopathy, mediastinal fibrosis, pulmonary fibrosis, anomalous pulmonary venous drainage, pulmonary venooclusive disease, pulmonary vasculitis, collagen vascular disease, congenital heart disease, pulmonary venous hypertension, interestitial lung disease, sleep-disordered breathing, apnea, alveolar hypoventilation disorders, chronic exposure to high altitude, neonatal lung disease, alveolar-capillary dysplasia, sickle cell disease, other coagulation disorders, chronic thromboemboli, pulmonary embolism, connective tissue disease, lupus, schitosomiasis, sarcoidosis, chronic obstructive pulmonary disease, emphysema, chronic bronchitis, pulmonary capillary hemangiomatosis; histiocytosis X, lymphangiomatosis or compressed pulmonary vessels;
(b) liver cirrhosis, or
(c) a urogenital system disorder selected from renal fibrosis, renal failure resulting from chronic kidney diseases or insufficiently, erectile dysfunction or female sexual dysfunction.

78. The method of claim 77, wherein the disease, health condition or disorder is pulmonary hypertension, pulmonary arterial hypertension, and associated pulmonary vascular remodeling, localized pulmonary thrombosis, right heart hypertophy, pulmonary hypertonia, primary pulmonary hypertension, secondary pulmonary hypertension, familial pulmonary hypertension, sporadic pulmonary hypertension, pre-capillary pulmonary hypertension, idiopathic pulmonary hypertension, thrombotic pulmonary arteriopathy, plexogenic pulmonary arteriopathy or chronic obstructive pulmonary disease, liver cirrhosis, renal fibrosis, renal failure resulting from chronic kidney diseases or insufficiently, erectile dysfunction or female sexual dysfunction.

79. The method of claim 78, wherein the disease, health condition or disorder is pulmonary hypertension, pulmonary arterial hypertension, and associated pulmonary vascular remodeling, pulmonary hypertonia, primary pulmonary hypertension, secondary pulmonary hypertension, familial pulmonary hypertension, sporadic pulmonary hypertension, pre-capillary pulmonary hypertension or idiopathic pulmonary hypertension.
